SEQUENCE LISTING (26 sequences)

SEQ ID NO: 1 - protein, 293 aa, synthetic construct
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 2 - protein, 293 aa, synthetic construct
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKNYRSTLTYGFNGNVTGDDTGKIGGLIGANVSIGHTLNYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 3 - protein, 277 aa, synthetic construct
CGSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRS

SEQ ID NO: 4 - protein, 551 aa, synthetic construct
CGSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRS

SEQ ID NO: 5 - protein, 825 aa, synthetic construct
CGSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRS

SEQ ID NO: 6 - protein, 1069 aa, synthetic construct
CGSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLA

SEQ ID NO: 7 - protein, 1247 aa, synthetic construct
SDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSGTSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSRRASACAGSAGSAGRSPRRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRS

SEQ ID NO: 8 - protein, 1247 aa, synthetic construct
SDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSGTSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGRRASACSAGSAGSAGSAGSAGSAGRSPRRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRS

SEQ ID NO: 9 - protein, 29 aa, synthetic construct
GSAGSAGSAGSAGSAGSAGSAGSAGSAGR

SEQ ID NO: 10 - protein, 39 aa, synthetic construct
GSAGSAGSAGRRASACSAGSAGSAGSAGSAGSAGRSPRR

SEQ ID NO: 11 - protein, 39 aa, synthetic construct
GSAGSAGSAGSAGSAGSAGSRRASACAGSAGSAGRSPRR

SEQ ID NO: 12 - DNA, 27 bp, synthetic construct
  source 1..27: mol_type = other DNA
gatccggtggtaccggcgagctcggta

SEQ ID NO: 13 - DNA, 27 bp, synthetic construct
  source 1..27: mol_type = other DNA
gatctaccgagctcgccggtaccaccg

SEQ ID NO: 14 - protein, 6 aa, synthetic construct
RRASAC

SEQ ID NO: 15 - DNA, 27 bp, synthetic construct
  source 1..27: mol_type = other DNA
agcgcctgcgcgggttctgctggttcc

SEQ ID NO: 16 - DNA, 27 bp, synthetic construct
  source 1..27: mol_type = other DNA
cgcacggcggctccctgcacttccggc

SEQ ID NO: 17 - DNA, 18 bp, synthetic construct
  source 1..18: mol_type = other DNA
tgcgagcgcctgcggtgg

SEQ ID NO: 18 - DNA, 18 bp, synthetic construct
  source 1..18: mol_type = other DNA
acgctcgcggacgccacc

SEQ ID NO: 19 - protein, 707 aa, synthetic construct
SDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSRRASACAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSGTGGPRRRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSRRASACAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRS

SEQ ID NO: 20 - protein, 707 aa, synthetic construct
SDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSRRASAAAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRSGTGGPRRRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSRRAAACAGSAGSAGRSDKIIHLTDDSFDTDVLKADGAILVDFWAEWSGPSKMIAPILDEIADEYQGKLTVAKLNIDQNPGTAPKYGIRGIPTLLLFKNGEVAATKVGALSKGQLKEFLDANLAGSAGSAGSAGSAGSAGSAGSAGSAGSAGRS

SEQ ID NO: 21 - protein, 38 aa, synthetic construct
GSAGSAGSAGRRASACSAGSAGSAGSAGSAGSAGRPRR

SEQ ID NO: 22 - protein, 38 aa, synthetic construct
GSAGSAGSAGSAGSAGSAGSRRASACAGSAGSAGRPRR

SEQ ID NO: 23 - protein, 38 aa, synthetic construct
GSAGSGASAGRRASACSAGSAGSAGSAGSAGSAGRPRR

SEQ ID NO: 24 - protein, 37 aa, synthetic construct
GSAGSGASAGSAGSAGSAGRRASACAGSAGSAGRPRR

SEQ ID NO: 25 - protein, 35 aa, synthetic construct
GSAGSAGSAGSAGSAGSAGSRRASAAAGSAGSAGR

SEQ ID NO: 26 - protein, 35 aa, synthetic construct
GSAGSAGSAGSAGSAGSAGSRRAAACAGSAGSAGR

(19) United States
(12) Patent Application Publication
(10) Pub. No.: US 2026/0072008 A1
(43) Pub. Date: Mar. 12, 2026

(54) METHOD OF CHARACTERISING A PEPTIDE, POLYPEPTIDE OR PROTEIN USING A NANOPORE

(71) Applicant: OXFORD UNIVERSITY INNOVATION LIMITED, Oxford, Oxfordshire (GB)

(72) Inventors: Pablo MARTIN-BANIANDRES, Oxford (GB); Wei-Hsuan LAN, Oxford (GB); Yujia QING, Oxford (GB); Mercedes ROMERO-RUIZ, Oxford (GB); Hagan BAYLEY, Oxford (GB); Sergi GARCIA-MANYES, London (GB)

(21) Appl. No.: 19/153,737

(22) PCT Filed: Feb. 7, 2024
(86) PCT No.: PCT/GB2024/050332

§ 371 Date: Aug. 5, 2025

(30) Foreign Application Priority Data
Feb. 7, 2023 (GB) 2301689.2

(51) Int. Cl. G01N 33/487; G01N 1/28; G01N 33/58; G01N 33/68

(52) U.S. Cl. CPC G01N 33/48721; G01N 1/28; G01N 33/58; G01N 33/6803; G01N 2440/14

(57) ABSTRACT

Provided herein are methods of characterising a peptide, polypeptide or protein and of characterising one or more proteoforms of a peptide, polypeptide or protein, using nanopores. Also provided herein are associated systems.

### FIELD

The invention relates to methods of characterising a peptide, polypeptide or protein using a nanopore. More specifically, the invention relates to the use of electroosmotic force to drive the movement of the peptide, polypeptide or protein through the nanopore in a linearised state; and taking measurements characteristic of the peptide, polypeptide or protein as the peptide, polypeptide or protein translocates the nanopore. The disclosure also relates to systems and associated kits and apparatuses for carrying out such methods.

### BACKGROUND

Single-molecule nanopore proteomics is gaining momentum. Nanopore sequencing of ultralong DNA and RNA has enabled biomedical applications that challenge short-read technologies. Nucleic acid sequencing has allowed the study of genomes and the proteins they encode; of the relationship between organisms through the discipline of evolutionary biology; and of the identity of organisms in a sample via metagenomics.

Despite significant recent progress in the characterisation of nucleic acids, methods to characterise other polymers such as peptide, polypeptide and proteins are less advanced, despite being of very significant biotechnological importance. For example, knowledge of a protein sequence can allow structure-activity relationships to be established and has implications in rational drug development strategies for developing ligands for specific receptors. Identification of post-translational modifications is also key to understanding the functional properties of many proteins. For example, the functional properties of most proteins are regulated by post-translational modifications (PTMs) of specific residues. Up to now, phosphorylation at serine, threonine or tyrosine is the most frequent experimentally determined PTM. Typically 30-50% of protein species are phosphorylated in eukaryotes, and some proteins may have multiple phosphorylation sites, serving to activate or inactivate a protein, promote its degradation, or modulate interactions with protein partners.

There is thus a pressing need for methods to characterise proteins and other polypeptides.

Known methods of characterising polypeptides include mass spectrometry and Edman degradation.

Protein mass spectrometry involves characterising whole proteins or fragments thereof in an ionised form. Known methods of protein mass spectrometry include electrospray ionisation (ESI) and matrix-assisted laser desorption/ionisation (MALDI). Mass spectrometry has some benefits, but results obtained can be affected by the presence of contaminants and it can be difficult to process fragile molecules without their fragmentation. Moreover, mass spectrometry is not a single molecule technique and provides only bulk information about the sample interrogated. Mass spectrometry is unsuitable for characterising differences within a population of polypeptide samples and is unwieldy when seeking to distinguish neighbouring residues. It is typically not possible to accurately map modifications that may be present in a peptide, polypeptide or protein using mass spectrometry, especially if the modifications are present on only a fraction of peptides, polypeptides or proteins in a sample.

Edman degradation is an alternative to mass spectrometry which allows the residue-by-residue sequencing of polypeptides. Edman degradation sequences polypeptides by sequentially cleaving the N-terminal amino acid and then characterising the individually cleaved residues using chromatography or electrophoresis. However, Edman sequencing is slow, involves the use of costly reagents, and like mass spectrometry is not a single molecule technique.

As such, there remains a pressing need for new techniques to characterise polypeptides, especially at the single molecule level. Single molecule techniques for characterising biomolecules such as polynucleotides have proven to be particularly attractive due to their high fidelity and avoidance of amplification bias.

Attempts have been made to characterise peptides, polypeptides and proteins using nanopores. In principle, such methods are attractive, as nanopore characterisation allows accurate measurements to be taken on the single molecule level in order to characterise analytes such as polymers. Nanopore sensing is an approach to analyte detection and characterization that relies on the observation of individual binding or interaction events between the analyte molecules and an ion conducting channel. Nanopore sensors can be created by placing a single pore of nanometre dimensions in an electrically insulating membrane. Electrical and/or optical measurements through the pore can be taken in the presence of analyte molecules. The presence of an analyte inside or near the nanopore alters the measurements obtained, thus allowing the identity of the analyte to be revealed.

Although methods to characterise analytes such as peptides, polypeptides and proteins are desirable, putting such methods into practice has been associated with significant challenges.

One approach that has been described is to rely on electrophoretic force to drive a charged polymer through a nanopore under the influence of an applied voltage.

For example, WO 2015/040423 describes methods for determining the presence, absence, number or position(s) of one or more post-translational modifications in a peptide, polypeptide or protein. The methods disclosed in WO 2015/040423 (the entire contents of which are incorporated herein by reference) involve attaching a highly charged DNA leader sequence to a peptide, polypeptide or protein in order to electrophoretically thread the peptide, polypeptide or protein through a nanopore. However, whilst this method has many advantages, some problems remain. For example, once the leader sequence exits the pore the leader has moved through the pore the residual movement of the peptide, polypeptide or protein may be irregular, which may hamper its analysis. Not all peptides, polypeptides or proteins naturally have appropriate sites for attachment of a leader and modifying them in order to allow such attachment may alter their properties away from those of the underlying native structure. Furthermore, the requirement to chemically attach a leader increases cost and experimental complexity, and may also involve the use of chemical reagents which alter the structure or properties of the underlying native peptide, polypeptide or protein.

Another approach that has been described is to rely on the use of processive enzymes such as unfoldases (e.g. ClpX9 or VATAN10) in order to ratchet a peptide, polypeptide or protein through a nanopore (e.g. see WO 2013/123379, the entire contents of which are incorporated herein by reference). However, whilst this approach has some advantages, the need to use such enzymes is associated with increased complexity, cost and experimental difficulty. Experimental conditions may not be compatible with the retention of enzymatic activity. Furthermore, many unfoldases are incapable of precise residue-by-residue translocation of polypeptides, and may not tolerate processing of large PTMs.

Accordingly, there remains a need for alternative and/or improved methods of characterising polymers such as peptides, polypeptides and proteins. The methods of the present invention are provided to address some or all of the difficulties outlined above.

### SUMMARY

In one aspect, the methods enable the characterisation of a peptide, polypeptide or protein of at least 25 amino acids in length. Such methods involves contacting the peptide, polypeptide or protein with an engineered protein nanopore. The nanopore has a first opening, a second opening and a solvent-accessible channel therebetween. The channel of the nanopore typically comprises one or more non-native charged moieties. The method is carried out under conditions such that an electroosmotic force across the nanopore causes the peptide, polypeptide or protein to translocate through the nanopore in a linearised state. One or more measurements characteristic of the peptide, polypeptide or protein are taken as the peptide, polypeptide or protein translocates the nanopore. In this manner, the peptide, polypeptide or protein is characterised.

In another aspect, the methods enable the characterisation of one or more proteoforms of a peptide, polypeptide or protein. Such methods involve contacting the peptide, polypeptide or protein with a nanopore. The method is carried out under conditions such that an electroosmotic force across the nanopore causes the peptide, polypeptide or protein to translocate through the nanopore in a linearised state. One or more measurements characteristic of the peptide, polypeptide or protein are taken as the peptide, polypeptide or protein translocates the nanopore. In this manner, the proteoforms of the peptide, polypeptide or protein are characterised.

Accordingly, provided herein is a method of characterising a peptide, polypeptide or protein at least 25 amino acids in length; comprising
contacting the peptide, polypeptide or protein with an engineered protein nanopore having a first opening, a second opening and a solvent-accessible channel therebetween;
under conditions such that an electroosmotic force across the nanopore causes the peptide, polypeptide or protein to translocate through the nanopore in a linearised state; and
taking one or more measurements characteristic of the peptide, polypeptide or protein as the peptide, polypeptide or protein translocates the nanopore;
thereby characterising the peptide, polypeptide or protein.

In some embodiments, said method is a method of characterising one or more proteoforms of said peptide, polypeptide or protein.

Also provided herein is a method of characterising one or more proteoforms of a peptide, polypeptide or protein; comprising
contacting the peptide, polypeptide or protein with a nanopore under conditions such that an electroosmotic force across the nanopore causes the peptide, polypeptide or protein to translocate through the nanopore in a linearised state; and
taking one or more measurements characteristic of the peptide, polypeptide or protein as the peptide, polypeptide or protein translocates the nanopore;
thereby characterising the proteoforms of the peptide, polypeptide or protein.

In some embodiments, said nanopore is a engineered protein nanopore having a first opening, a second opening and a solvent-accessible channel therebetween. In some embodiments, the nanopore is a mutant protein nanopore and the channel of said nanopore comprises one or more non-native charged moieties. In some embodiments, said peptide, polypeptide or protein is at least 25 amino acids in length.

In some embodiments, said proteoforms of said peptide, polypeptide or protein that are characterised are selected from proteoforms corresponding to modifications in the genome, modifications in the RNA, modifications during translation and modifications at the protein level; somatic mutations, long-range genome rearrangements; recombinations (e.g. V (D) J recombinations), somatic hypermutations, alternative splicings, RNA base editing modifications, frameshift modifications, codon reassignments, translational bypass modifications, translational errors, modifications arising from proteolytic processing, protein splicing modifications, post-translational modifications (PTMs) and chemical rearrangements. In some embodiments, characterising said proteoforms comprises detecting and/or characterising one or more post-translational modifications. In some embodiments, characterising said proteoforms comprises detecting and/or characterising one or more RNA splicing sites.

In some embodiments, said method is a method of determining the presence, absence, number, position, or identity of one or more post-translational modifications at one or more sites within the peptide, polypeptide or protein. In some embodiments, said one or more sites are at least 25 amino acids from the N-terminus and/or at least 25 amino acids from the C terminus of said peptide, polypeptide or protein.

In some embodiments, characterising said proteoforms comprises detecting and/or characterising (preferably by determining the presence, absence, number, position, or identity) of two or more post-translational modifications. In some embodiments, said two or more post-translational modifications are separated in said peptide, polypeptide or protein by at least 50, at least 100, at least 150 or at least 200 amino acids.

In some embodiments, said nanopore is modified to increase the ion selectivity of the nanopore. In some embodiments, the channel of the nanopore comprises one or more non-native charged moieties having a charged side chain. In some embodiments, the one or more non-native charged moieties comprise one or more positively charged amino acids and said one or more positively charged amino acids increase the anion selectivity of the nanopore.

In some embodiments, said nanopore is a transmembrane β-barrel protein nanopore.

In some embodiments, said peptide, polypeptide or protein has a net charge of between about −10 and about +10 per 50 amino acids. In some embodiments, said peptide, polypeptide or protein has a net charge of between about −5 and about +5 per 30 amino acids.

In some embodiments, said method comprises contacting the peptide, polypeptide or protein with a chaotropic agent prior to the translocation of the peptide, polypeptide or protein through the nanopore. In some embodiments, said method is carried out in the presence of a chaotropic agent. In some embodiments, said chaotropic agent is a denaturant. In some embodiments, said chaotropic agent is selected from guanidinium salts, guanidinium isothiocyanate, urea and thiourea.

In some embodiments, said method is conducted between about pH 4 and about pH 10.

In some embodiments, said method comprises applying a voltage during said method, and the voltage applied varies during the method. In some embodiments, the method comprises applying a voltage ramp during the method.

In some embodiments, said peptide, polypeptide or protein comprises a concatamer of two or more peptides, polypeptides and/or proteins. In some embodiments, the peptides, polypeptides and/or proteins in said concatamer are attached together by one or more linkers.

In some embodiments, said peptide, polypeptide or protein comprises or consists of a complete intact protein.

In some embodiments, said method comprises characterising a plurality of peptides, polypeptides or proteins.

In some embodiments, the peptide, polypeptide or protein is not attached to a charged leader. In some embodiments, the peptide, polypeptide or protein is not attached to (a) a polynucleotide leader or (b) an anionic peptide such as a poly-aspartate, poly-glutamate or poly(aspartate/glutamate) leader.

In some embodiments, a motor protein is not used to control the translocation of the peptide, polypeptide or protein through the nanopore.

In some embodiments, characterising said polypeptide or said proteoforms of said peptide, polypeptide or protein comprises detecting the number, position and/or nature of modifications in said peptide, polypeptide or protein as the peptide, polypeptide or protein translocates through the nanopore.

In some embodiments, the provided method is a method of characterising one or more post-translational modifications in a peptide, polypeptide or protein; comprising
contacting the peptide, polypeptide or protein with a label capable of binding to said one or more post-translational modifications;
contacting the peptide, polypeptide or protein with a nanopore under conditions such that an electroosmotic force across the nanopore causes the peptide, polypeptide or protein to translocate through the nanopore in a linearised state; and
taking one or more measurements characteristic of the label as the peptide, polypeptide or protein translocates the nanopore;
thereby characterising the one or more post-translational modifications of the peptide, polypeptide or protein.

Also provided herein is a system, comprising
an engineered protein nanopore having a first opening, a second opening and a solvent-accessible channel therebetween; and
a peptide, polypeptide or protein at least 25 amino acid in length;
wherein said nanopore and/or said peptide, polypeptide or protein is present in a medium comprising a chaotropic agent.

In some embodiments, the channel of the nanopore comprises one or more non-native charged moieties. In some embodiments, said nanopore is comprised in a membrane and said system further comprises means for detecting electrical and/or optical signals across said membrane. In some embodiments, said peptide, polypeptide or protein comprises one or more post-translational modifications and/or one or more RNA splicing sites. In some embodiments, said system is configured such that when the peptide, polypeptide or protein is contacted with the nanopore an electroosmotic force across the nanopore is capable of causing the peptide, polypeptide or protein to translocate through the nanopore in a linearised state.

### BRIEF DESCRIPTION OF THE FIGURES

FIGS. 2 to 12 relate to the experiments described in example 1.

FIGS. 13 to 19 relate to the experiments described in example 2.

FIG. 1. A non-limiting schematic depicting the methods of the present invention. The capture, unfolding, and single-file translocation of long (>1000 residues), underivatized polypeptide chains through protein nanopores under a constant electroosmotic force has been demonstrated. Various post-translational modifications (PTMs) located deep within the polypeptide chains can be identified by monitoring a transmembrane ionic current during translocation. Key attributes of the claimed approach include: (i) Full-length reads of long polypeptide chains can be generated; (ii) the polypeptide analytes need not be covalently modified before analysis; (iii) PTMs may be mapped within entire, individual polypeptide chains, rather than (e.g.) presented as an ensemble of disconnected peptide fragments; (iv) widely separated PTMs located deep within individual polypeptide chains can be mapped; (v) the approach is amenable to commercial nanopore devices for fast, highly parallel, inexpensive proteomic studies; and (vi) single-cell proteomics is achievable by the approach.

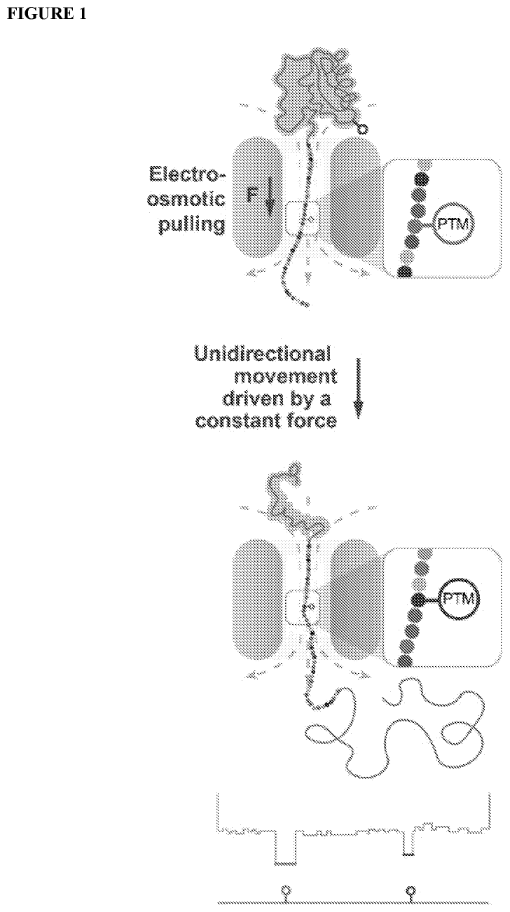

FIG. 2. Non-limiting example of electroosmosis-driven translocation of thioredoxin-linker concatamers through a protein nanopore. Electroosmotic flow (EOF) in a charge-selective nanopore, (labelled (NN-113R)₇), drives the sequential co-translocational unfolding of polypeptide (exemplified as thioredoxin (Trx)) units within a polyprotein of >1000 amino acids.

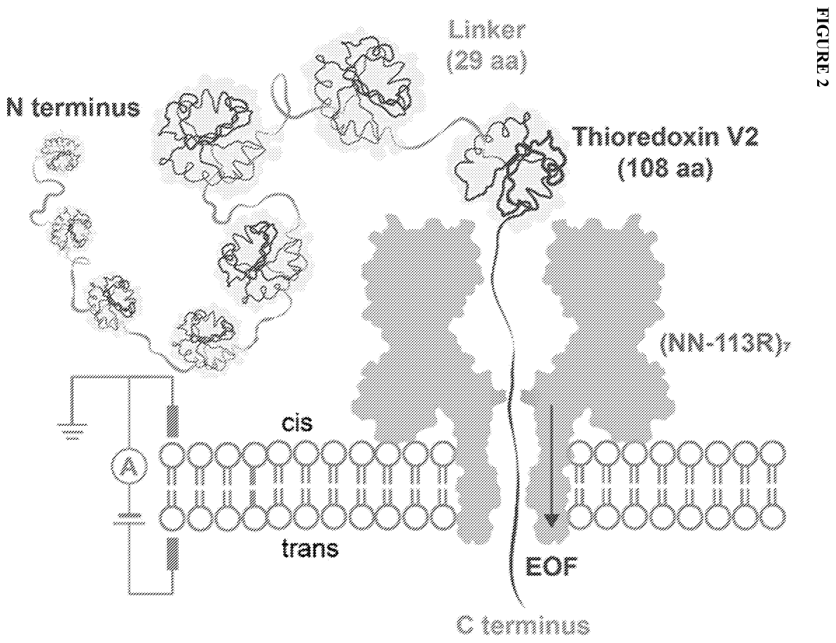

FIG. 3. SDS-polyacrylamide gel showing a Trx-linker dimer (28 kDa), tetramer (55 kDa), hexamer (82 kDa), and octamer (108 kDa), described in the example

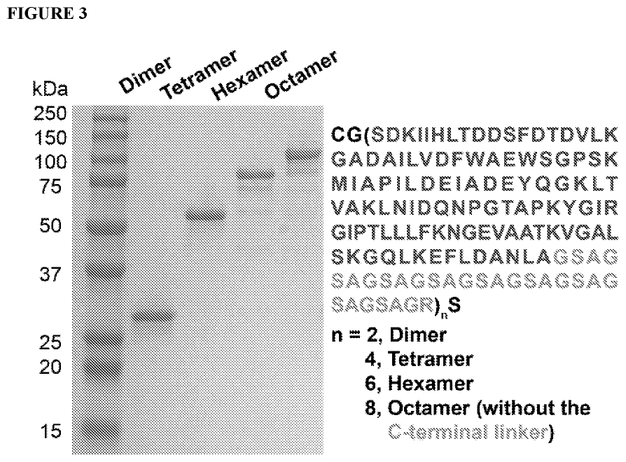

FIG. 4. Current recordings for the C-terminus-first translocation of a dimer, a tetramer, a hexamer and an octamer without post-acquisition filtering. The repeating features A are indicated by orange and blue bars (in original colour image).

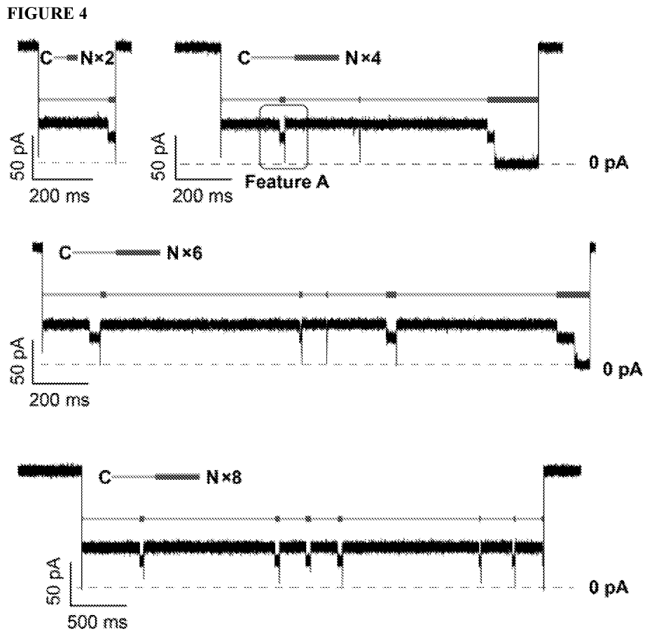

FIG. 5. Zoom-in of the repeating feature A boxed in blue in FIG. 4 without post-acquisition filtering. Three levels are assigned as: A1. a linker within the pore; A2, A3. different segments of partly unfolded Trx within the pore. Conditions in c and d: 750 mM GdnHCl, 10 mM HEPES, 5 mM TCEP, pH 7.2, Trx-linker concatamers (cis) (dimer: 2.23 μM; tetramer: 0.63 μM; hexamer: 0.25 μM; octamer: 0.81 μM), +140 mV (trans), 24±1° C.

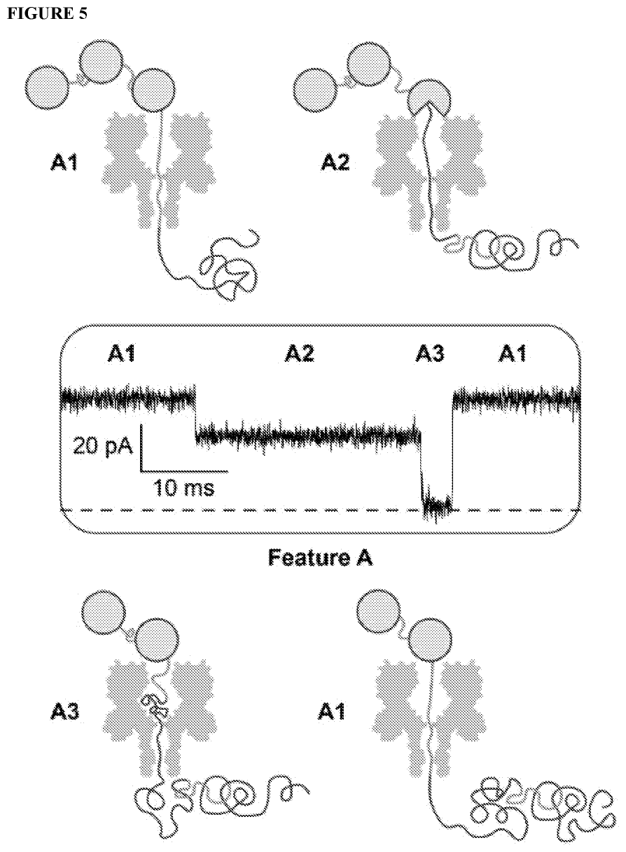

FIG. 6. Non-limiting example of detection of PTMs in protein concatamers traversing a nanopore driven by electroosmotic flow. The Trx-linker nonamers tested (SEQ ID NOs 10 and 11) contained a RRASAC sequence within the central linker, which was post-translationally phosphorylated (purple), S-glutathionylated (green) or glycosylated (yellow) (coloured in original image).

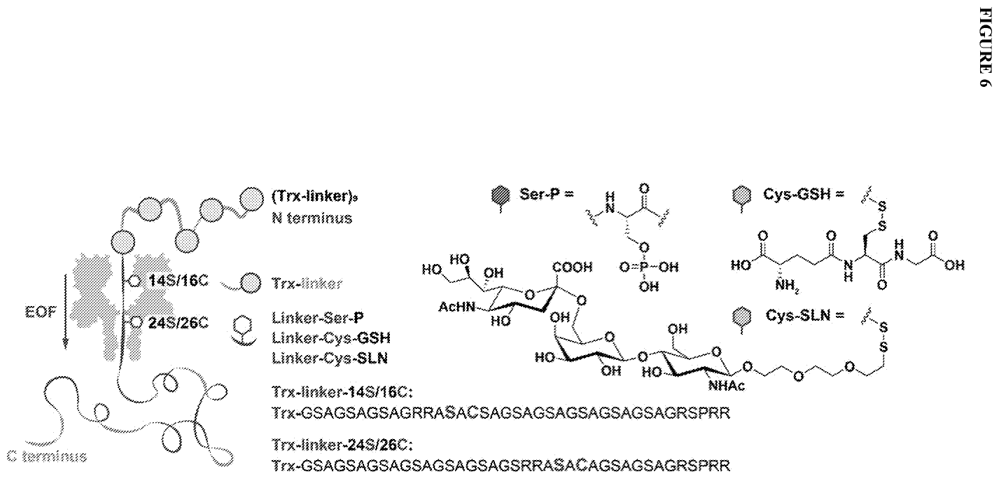

FIG. 7. Left: Recordings of C terminus-first translocation events of Trx-linker nonamers showing a distinct Level A1 (boxed in purple, green or yellow) in the presence of a PTM compared to the unmodified A1 (orange dash) (coloured in original image). Traces have been filtered at 2 kHz; transient A3 levels were truncated and therefore deviated from ˜0 pA. The A3 produced by the translocation of an unmodified unit before the modified linker is indicated with a blue arrow and each of the features A recorded is indicated by orange and blue bars. Right: Scatter plots of I_{RMS} and ΔI_{res %} for individual translocation events, ΔI_{res %}=<I_{res %} (A1, Trx-linker)>−I_{res %}(A1, Trx-linker+PTM), where <I_{res %}(A1, Trx-linker)> is the mean I_{res %} value of the remaining A1 levels for unmodified repeat units within an individual translocation event. Conditions: 375 mM GdnHCl, 375 mM KCl, 10 mM HEPES, pH 7.2, 1.2 μM Trx-linker nonamer (cis), 140 mV (trans), 24±1° C.

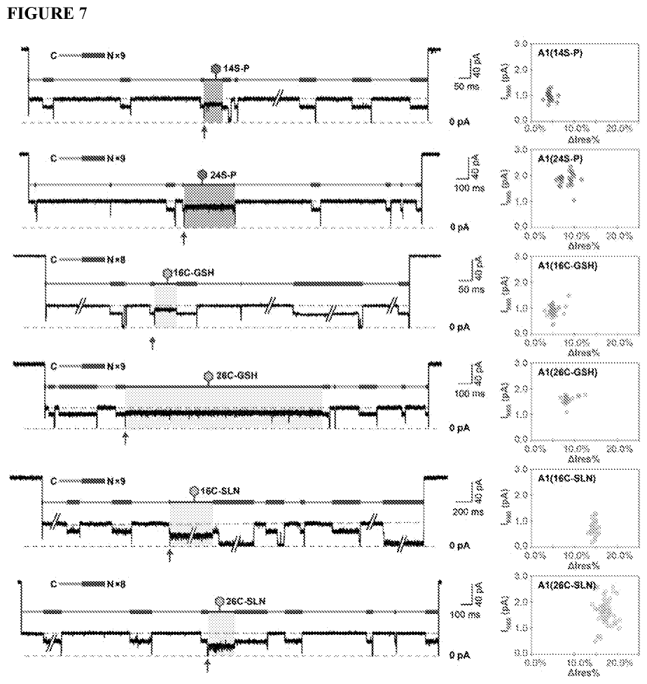

FIG. 8. Repeating current features recorded during electroosmosis-driven concatamer translocation through a nanopore. Two repeating current features, A or B, were recorded with Trx-linker octamers pre-treated with 5 mM tris(2-carboxyethyl) phosphine (TCEP) for 10 min prior to their addition to the cis compartment of the recording chamber. Conditions: 750 mM GdnHCl, 10 mM HEPES, pH 7.2, 0.81 μM Trx-linker octamer (cis), 5 mM TCEP, +140 mV (trans), 24±1° C.

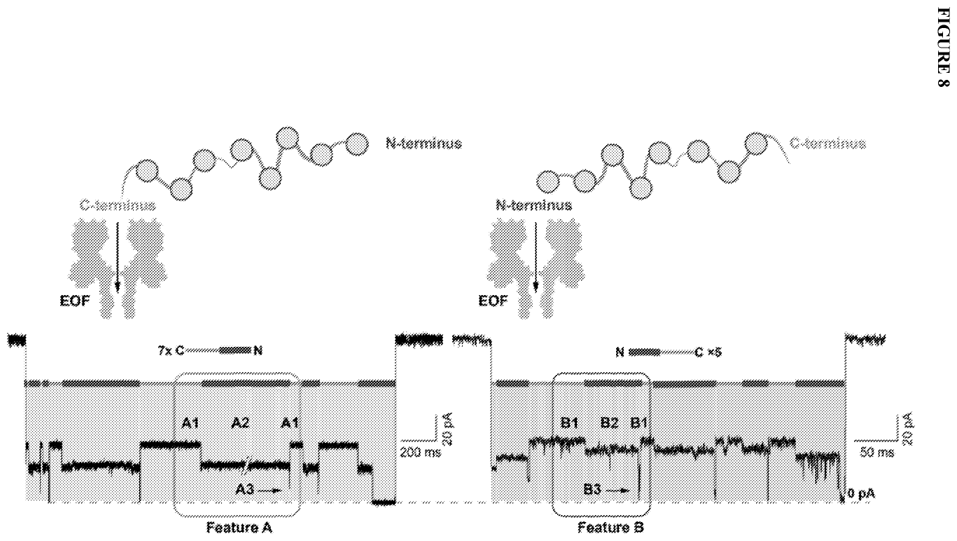

FIG. 9. Without the TCEP pre-treatment, features A were always seen before features B when they occurred together within a single translocation event. The first two levels (B1 and B2) in features B have larger noise and higher I_{res %} compared to A1 and A2 recorded within a single translocation event with a single pore (A1: I_{res %}=35±1%, I_{RMS}=1.1±0.1 pA, N=25; A2: I_{res %}=21±1%, I_{RMS}=1.5±0.2 pA, N=25; B1: I_{res %}=38±1%, I_{RMS}=1.7±0.4 pA, N=39; B2: I_{res %}=32±1%, I_{RMS}=2.0±0.5 pA, N=39). The translocating molecules, which gave sequential A and B features, were assigned as dimers of octamers linked by a disulfide bond between the two N-terminal cysteines. Therefore, in the unlinked molecules (see FIG. 8), C terminus-first translocation occurred when features A were observed and N terminus-first translocation occurred when features B were observed. The repeating features are indicated by orange and blue bars (coloured in original image). Conditions: 750 mM GdnHCl, 10 mM HEPES, pH 7.2, 0.81 μM Trx-linker octamer (cis), +140 mV (trans), 24±1° C. All traces were filtered at 2 kHz for clarity; transient A3 levels were truncated and therefore deviated from ˜0 pA

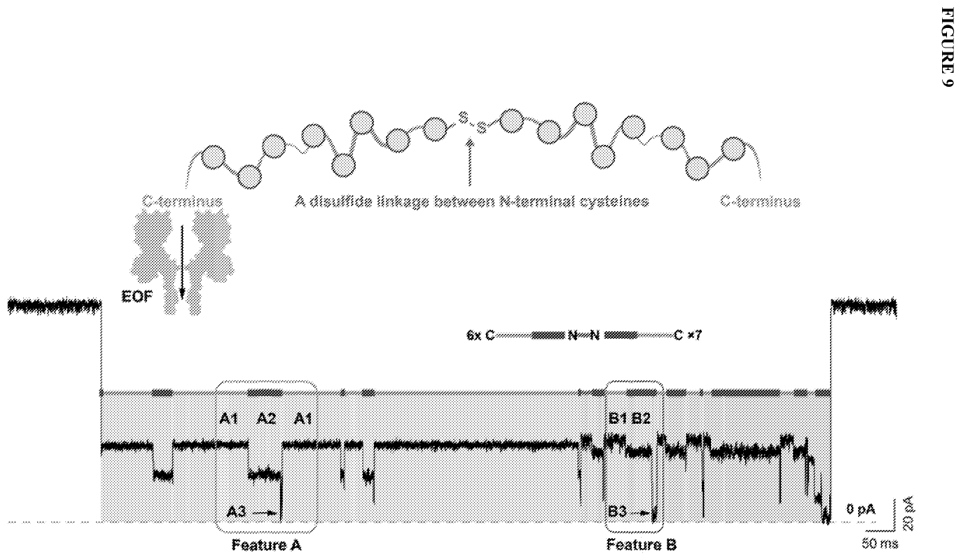

FIG. 10. Non-limiting example of electroosmosis-driven translocation of Trx-linker octamers through a nanopore. Electroosmosis-driven translocation of Trx-linker octamers through a nanopore. a-e, Current traces for the translocation of Trx-linker octamers in the presence of 750 mM GdnHCl (a), 1.5 M GdnHCl (b), 3 M GdnHCl (c) without post-acquisition filtering, 2 M urea (d) or no denaturant (e) with 2 kHz post-acquisition filtering. Current features for subunit-by-subunit translocation were lost at 3 M GdnHCl (c). The mean number of features A recorded per concatemer is (a) ˜4, (b) ˜3, (c) 0, (d) ˜4, and (e) ˜4. Conditions: 10 mM HEPES, pH 7.2, 0.81 μM Trx-linker octamer (cis), +140 mV (trans), 24±1° C., with (a) 750 mM GdnHCl; (b) 1.5 M GdnHCl; (c) 3 M GdnHCl; (d) 2 M urea and 750 mM KCl; (e) 750 mM KCl.

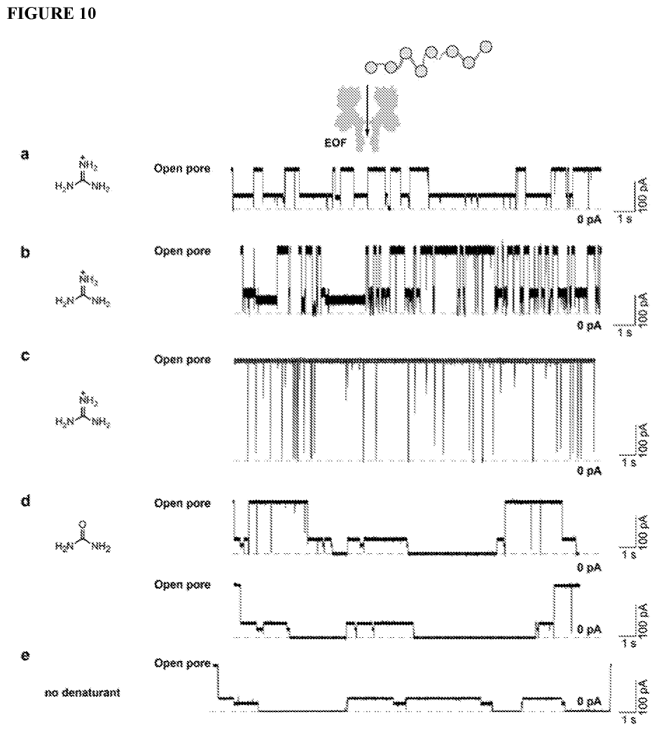

FIG. 11. Non-limiting example of identification and positional discrimination of PTMs in protein concatamers by electroosmotic flow through a nanopore. Protein nonamers containing a single PTM (See FIG. 2 for protein sequences and PTM structures) were tested. a-c, Scatter plots of I_{RMS} and ΔI_{res %} showing positional discrimination of a phosphorylated serine, a glutathionylated cysteine, or a glycosylated cysteine at sites 10 aa apart (ΔI_{res %}=<I_{res %}(A1, Trx-linker)>−I_{res %} (A1, Trx-linker+PTM), where <I_{res %}(A1, Trx-linker)> is the mean I_{res %} value of A1 levels of an unmodified unit within a single translocation event. Conditions: 375 mM GdnHCl, 375 mM KCl, 10 mM HEPES, pH 7.2, 1.2 μM Trx-linker nonamer (cis), +140 mV (trans), 24±1° C. d-f, Overlaid scatter plots of I_{RMS} and ΔI_{res %} showing discrimination between phosphorylated and glycosylated populations, glutathionylated and glycosylated populations, and overlaps between phosphorylated and glutathionylated populations.

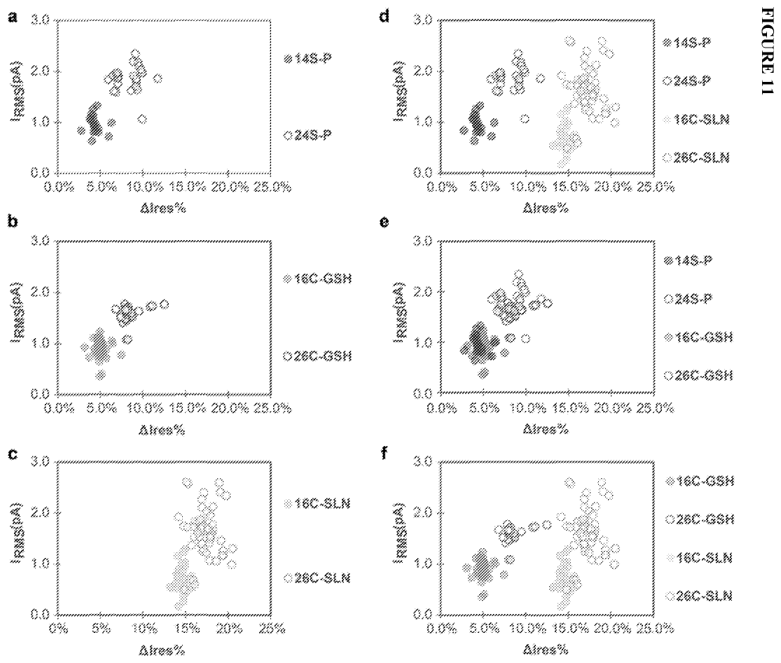

FIG. 12. Positions of modification sites during translocation through an αHL pore. a, The Trx-linker nonamers tested contained a RRASAC sequence within the central linker, which was post-translationally modified (hexagon). In a C-terminus-first threading configuration, as shown, the 14S/16C modification sites would be located closer to the cis opening of the αHL pore than the 24S/26C pair, when translocation is paused with a Trx unit at the cis mouth of the pore. b-d, Depending on the degree of extension of the polypeptide chain under the EOF (3.5 Å per aa when fully extended, 1.7-2.2 Å per aa under ˜5-10 pN²²), the 14S/16C and 24S/26C sites could be located at different positions within an αHL pore. Assuming that the N-terminal residue of the linker is at the cis opening of the pore when the translocation is arrested by a folded Trx unit, the modified linker (red; coloured in original image) might fully span the αHL pore (b) or occupy only a part of the nanopore (c,d). When the 24S/26C sites are located nearer the central constriction of the αHL pore (c,d), a PTM at 24S/26C would produce a larger current blockade than that at 14S/16C (PTM=Ser-P, Cys-GSH, Cys-SLN), which is what was observed (FIG. 10b). Given that the applied potential drops mostly across the transmembrane β barrel²³, the current difference between 14S/16C+PTM and 24S/26C+PTM is likely to be larger in c than in d.

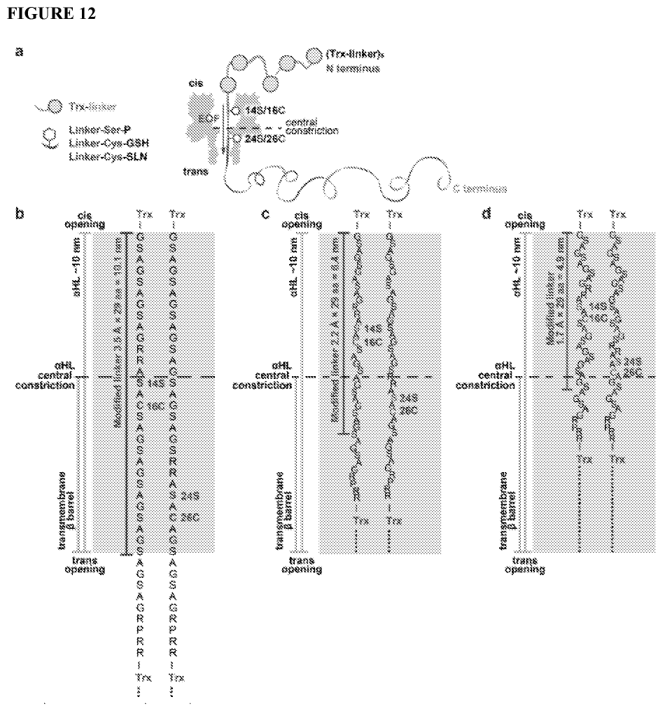

FIG. 13. Detection of serine phosphate bound to Phos-tag in a polypeptide chain.

a, Monitoring the Trx-linker pentamer traversing the α-hemolysin nanopore (NN-113R)₇. The Trx-linker pentamer contained two RRAS sequences within the second and fourth linkers, which were phosphorylated on serine. b, Left: Phosphorylated serine residues (Ser-P) 274 aa apart on a Trx-linker pentamer were detected. Level A1 for the linker between Trx unit 3 and unit 4 showed a slightly lower I_{res %} compared to unmodified segments, such as the linker between first and second Trx. This difference was attributed to the additional amino acid sequence in the third linker (Table S1). Right: Scatter plots of I_{r.m.s.} and ΔI_{res %} for individual translocation events, ΔI_{res %}=<I_{res %} (A1, Trx-linker)>−I_{res %} (A1-P), where <I_{res %} (A1, Trx-linker)> is the mean I_{res %} value of the remaining A1 levels for unmodified repeat units within an individual translocation event. If there were two Ser-P detected in different segments within a single translocation event, they were analyzed individually. c, Left: Phos-tag-acrylamide dizinc complexes bound to serine phosphate produced alternating current levels (A1-P-PAZn₂). Right: Scatter plots of I_{r.m.s.} and ΔI_{res %} for individual translocation events. Data points in light green are the I_{r.m.s.} and ΔI_{res %} values for the higher level of the two-level A1 state (A1-P-PAZn₂-H), while data points in dark green are the I_{r.m.s.} and ΔI_{res %} values for the lower level of the two-level A1 state (A1-P-PAZn₂-L). If there were two A1-P-PAZn₂ detected in different segments in a single translocation event, they were analyzed individually. d, Left: Phos-tag dizinc complexes bound to serine phosphate generated alternating current levels (A1-P-PZn₂). Right: Scatter plots of I_{r.m.s.} and ΔI_{res %} for individual translocation events. Data points in light purple are the I_{r.m.s.} and ΔI_{res %} values for the higher level of the two-level A1 state (A1-P-PZn₂-H), while data points in dark purple are the I_{r.m.s.} and ΔI_{res %} values for the lower level of the two-level A1 state (A1-P-PZn₂-L). If there were two A1-P-PZn₂ detected in different segments in a single translocation event, they were analyzed individually. Conditions in b: 10 mM HEPES, pH 7.2, 750 mM GdnHCl, 2.37 μM Trx-linker pentamer (cis), +140 mV (trans), 23±1° C. Conditions in c: 10 mM HEPES, pH 7.2, 750 mM GdnHCl, 2.37 μM Trx-linker pentamer (cis), 118.5 μM Phos-tag-acrylamide (cis), 237 μM ZnCl₂ (cis), +140 mV (trans), 23±1° C. Conditions in d: 10 mM HEPES, pH 7.2, 750 mM GdnHCl, 2.37 μM Trx-linker pentamer (cis), 237 μM Phos-tag (cis), 474 μM ZnCl₂ (cis), +140 mV (trans), 23±1° C.

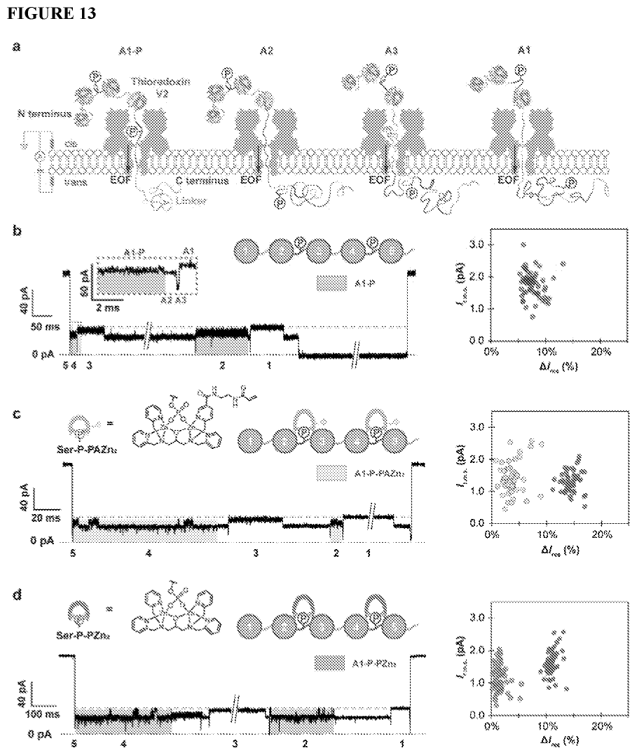

FIG. 14. Detection of phosphorylation and glutathionylation in a Trx-linker pentamer in the presence of Phos-tag. a, Monitoring the phosphorylated and glutathionylated Trx-linker pentamer during translocation through a (NN-113R)7 αHL nanopore. The pentamer is phosphorylated on Ser-24 (Ser-P) of the second linker and glutathionylated on the Cys-26 (Cys-GS) of the fourth linker. The blockades and noises from Ser-P and Cys-GS cannot be readily discriminated. b, PAZn₂ produced an additional current feature when bound to Ser-P. Conditions in a: 10 mM HEPES, pH 7.2, 750 mM GdnHCl, 2.37 μM Trx-linker pentamer (cis), +140 mV (trans), 23±1° C. Conditions in b: 10 mM HEPES, pH 7.2, 750 mM GdnHCl, 2.37 μM Trx-linker pentamer (cis), 118.5 μM Phos-tag-acrylamide (cis), 237 μM ZnCl2 (cis), +140 mV (trans), 23±1° C.

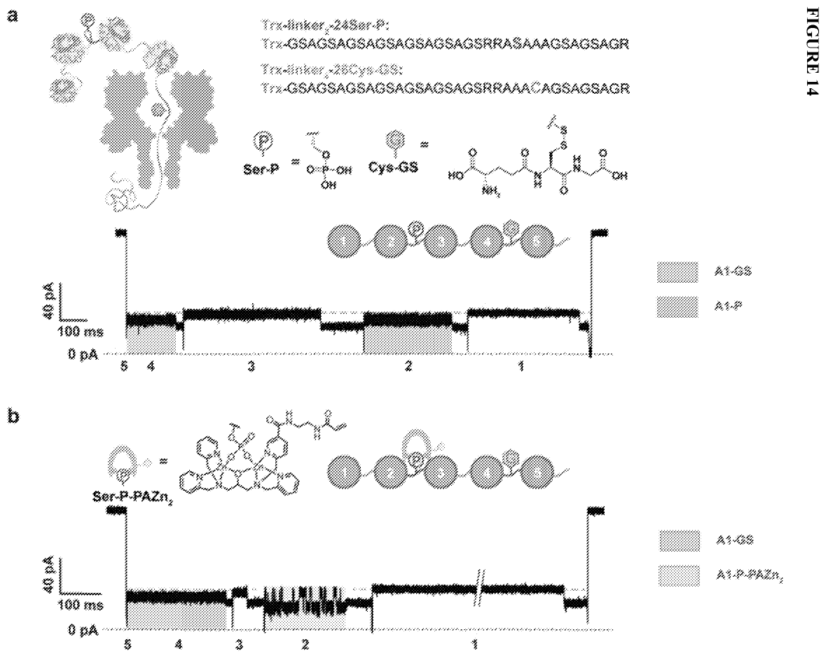

FIG. 15. An SDS-polyacrylamide gel of the Trx-linker pentamer. (Trx-linker) 1,3,5 (Trx-linker-24S26C)_{2,4}: 71 kDa.

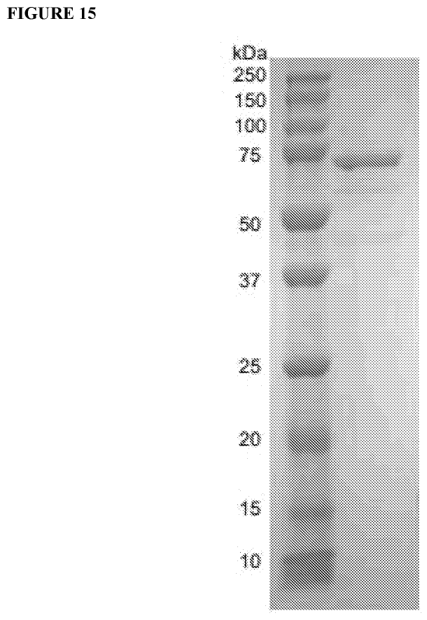

FIG. 16. Figure S2. ESI LC-MS characterization of Trx-linker pentamers. LC-MS chromatograms (top) and deconvoluted ESI-MS spectra (bottom). (Trx-linker) 1.3.5 (Trx-linker-24S26C)_{2,4}: mass=71197 Da (calc) and 71195 Da (obs); (Trx-linker)_{1,3,5} (Trx-linker-S24P)_{2,4}: mass=71356 Da (calc) and 71356 Da (obs); (Trx-linker)_{1,3,5} (Trx-linker-24S)₂ (Trx-linker-26C)₄: mass=71149 Da (calc) and 71148 Da (obs); (Trx-linker)_{1,3,5} (Trx-linker-S24P)₂ (Trx-linker-C26GS)₄: mass=71534 Da (calc) and 71534 Da (obs).

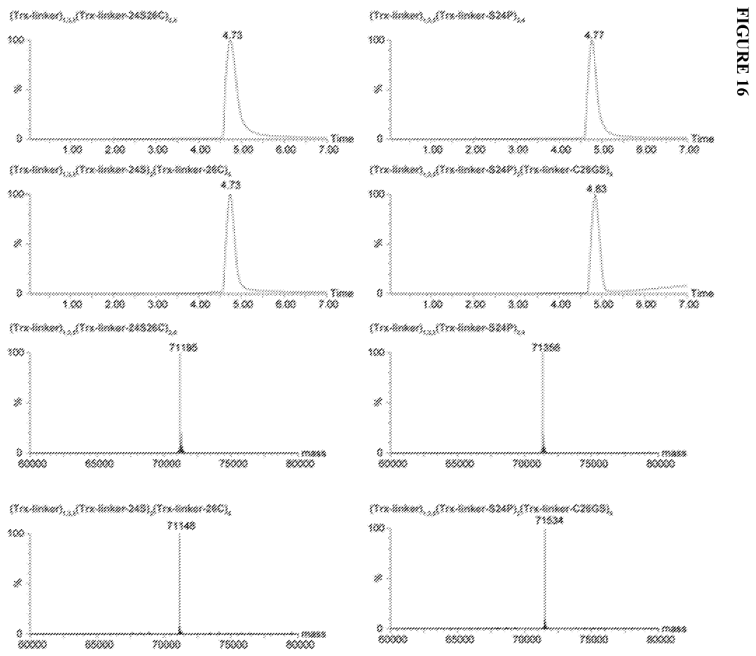

FIG. 17. Fractions of phosphorylated linkers detected in the PAZn₂-bound state, tested in two molar equivalents of Phos-tag-acrylamide dizinc complexes (10 eq. and 50 eq.).

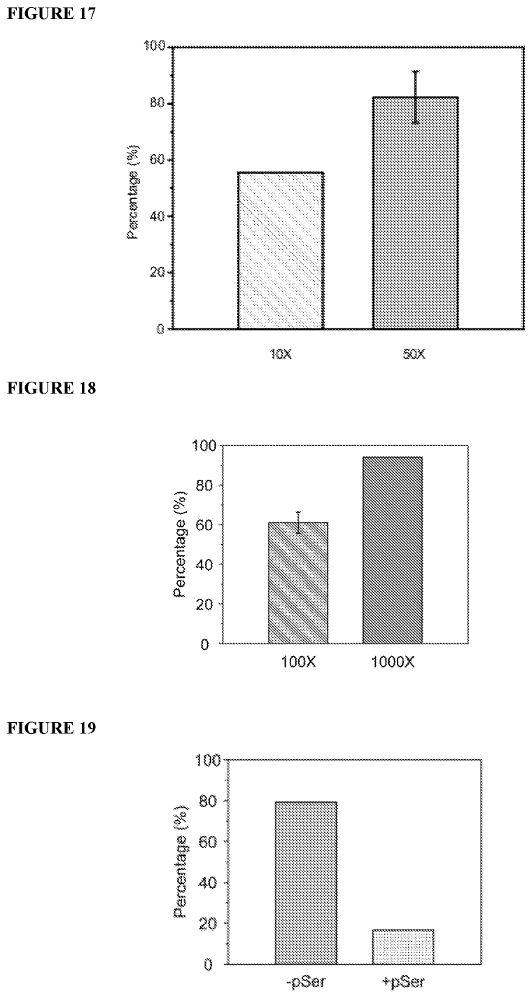

FIG. 18. Fractions of phosphorylated linkers detected in the PZn₂-bound state, tested in two molar equivalents of Phos-tag-acrylamide dizinc complexes (100 eq. and 1000 eq.).

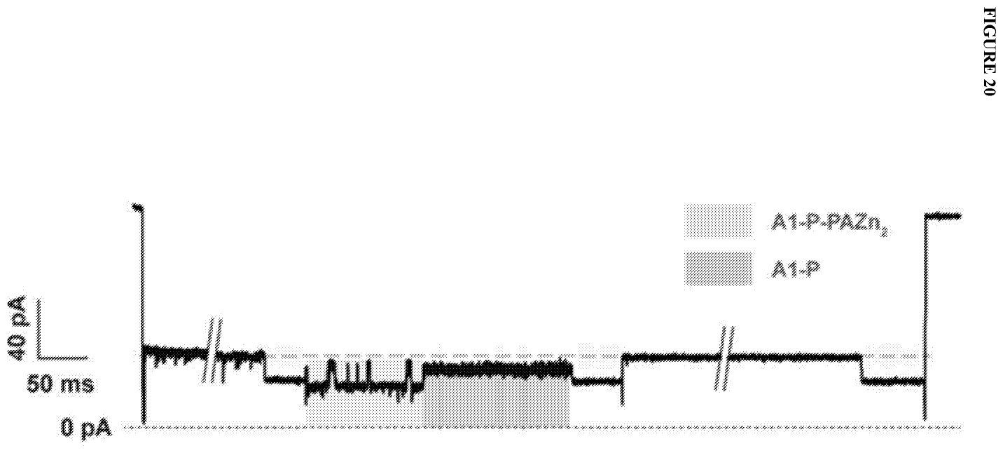

FIG. 19. Fractions of events containing at least one level A1-P-PAZn in the absence and presence of competing phosphoserine

FIG. 20. A current trace showing transition between level A1-P-PAZn₂ and level A1-P when a phosphorylated segment was inside the (NN-113R)₇ nanopore.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

It should be appreciated that “embodiments” of the disclosure can be specifically combined together unless the context indicates otherwise. The specific combinations of all disclosed embodiments (unless implied otherwise by the context) are further disclosed embodiments of the claimed invention.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a motor protein” includes two or more such proteins, reference to “a helicase” includes two or more helicases, reference to “a monomer” refers to two or more monomers, reference to “a pore” includes two or more pores and the like.

All publications, patents and patent applications cited herein, whether supra or infra, are hereby incorporated by reference in their entirety.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L a-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as B-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypeptides that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” has at least 50%, 60%, 70%, 80%, 90%, 95% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

### Movement Under Electroosmotic Force

The methods provided herein involve the movement of a peptide, polypeptide or protein through a nanopore under an electroosmotic force. The peptide, polypeptide or protein is characterised as it moves through a nanopore.

In contrast to methods which seek to control the movement of peptides, polypeptides and proteins using electrophoresis, the methods provided herein relate to controlling the movement of a peptide, polypeptide or protein through a nanopore using electroosmosis.

Peptides, polypeptides and proteins are typically substantially uncharged or have low net charge and/or charge density, and/or are irregularly charged. In other words, charge distribution in a peptide, polypeptide or protein is typically low and/or irregularly distributed along the length of a target polypeptide. As set out in Table 1 above, some amino acids which are comprised in target polypeptides are polar, and some are non-polar. Some are positively or negatively charged under physiological conditions, others are uncharged under physiological conditions but may be charged under the conditions under which methods such as those disclosed herein are carried out, and yet others are uncharged under all relevant conditions. The distribution of amino acids in the target polypeptide is a function of the exact analyte being characterised in the disclosed methods and thus may not be known by the user in advance.

In known methods of polypeptide analysis which rely on electrophoretic movement of polypeptides through a nanopore, this irregular charge along a target polypeptide may present difficulties, because the electrophoretic force acting on the polypeptide will vary as the polypeptide strand moves through the nanopore. In consequence, the rate of movement of the polypeptide through the nanopore may be unpredictable, which hampers accurate characterisation. For example, it may be difficult to distinguish two identical amino acids which move quickly through a pore from one amino acid which moves more slowly. The low average charge density of target polypeptides is one reason that known methods for characterising polynucleotides and analogues thereof (such as PNA; peptide nucleic acid) are typically unsuitable for the accurate characterisation of target polypeptides.

Electroosmosis (also referred to as electroosmotic force) is the motion of liquid induced by an applied potential across a porous material, such as across a nanopore as described herein. Electroosmotic flow is caused by the Coulomb force induced by an electric field on net mobile electric charge in a solution. Because the chemical equilibrium between a surface and an electrolyte solution typically leads to the interface acquiring a net fixed electrical charge, a layer of mobile ions, known as an electrical double layer or Debye layer, forms in the region near the interface. When an electric field is applied to the fluid (usually via electrodes placed at inlets and outlets), the net charge in the electrical double layer is induced to move by the resulting Coulomb force. The resulting flow is termed electroosmotic flow.

Critically, the liquid that moves under an electroosmotic force can carry a particle. The particle itself need not be charged. Thus, as described in more detail herein, the electroosmotic movement of a liquid such as an aqueous solvent (e.g. buffered aqueous solution) through a nanopore can carry an uncharged (or weakly and/or irregularly charged) particle through the nanopore, such as a peptide, polypeptide or protein particle.

By contrast, electrophoresis relates to the movement of a charged particle under the influence of an electric field. Those skilled in the art appreciate that there is a profound difference between systems which rely on electrophoresis in order to bias the movement of a polymer through a pore, and those which rely on electroosmosis. In particular, electrophoretic movement of a peptide, polypeptide or protein is thus typically ineffective.

In other words, an uncharged particle (or weakly charged particle) may be subjected to electroosmotic force, whereas it is not subjected to electrophoretic force. Thus, it is not possible to electrophoretically move an uncharged polymer with respect to a nanopore by applying an electric field, e.g. by applying a voltage potential across the nanopore. By contrast, it is possible to move an uncharged particle through a nanopore under the influence of an electroosmotic force.

The electroosmotic movement of small cyclodextrin molecules through protein nanopores (and the binding of such molecules thereto) was first demonstrated by the inventors in 2003 (Gu et al, PNAS 100 (26) 2003). More recently, efforts have been made to exploit electroosmotic forces to transport more complex species through nanopores.

One approach that has been attempted is to focus solely on very short peptides where structural complexity (including secondary and tertiary structure) is minimised in order to facilitate nanopore interactions. Under these conditions electroosmotic force has been shown to allow the translocation of such peptides to be detected. However, the detailed characterisation of such peptides during their translocation, and the detection of longer peptides which natively may have significant secondary/tertiary structure has remained an unmet challenge.

In seeking to address longer peptides, a second approach that has been described in art is to seek to translocate folded peptides through large nanopores under electroosmotic force. This approach has generally proven to be unsuccessful, but even where this has been described such methods cannot be used to characterise details of the peptides, such as its sequence or proteoforms of such peptides with characteristic features buried in the peptide structure, such as PTMs which are located far from the N- or C-termini of such peptides. The detailed characterisation of long peptides, polypeptides and proteins using electroosmotic force has not been demonstrated.

The present inventors have sought to address these issues. Surprisingly, it has now been shown that even long peptides can be unfolded (linearised) and translocated through nanopores in order to allow their detailed characterisation under an electroosmotic force. The methods thus arising have profound implications for polypeptide analysis. In particular, the methods can be used to characterise complete intact proteins. This is a significant advantage compared to methods which involve the fragmenting of proteins prior to their analysis. In particular, problems associated with reassembly of the fragments in order to map the protein structure are avoided. The methods are thus simpler and more accurate than methods that rely on protein fragmentation.

Contrary to methods which seek only to probe short peptides or small molecules, the disclosed methods can be used to characterise long peptides, including concatamers of proteins. This is described in more detail herein. Contrary to methods which merely detect crude signals arising from the interaction of folded peptides with a nanopores, the disclosed methods allow detailed characterisation of the polypeptide as it moves with respect to the nanopore, including characterisation of PTMs that may be buried in the native (folded) protein structure. Contrary to methods which rely on electrophoresis in order to achieve peptide translocation (e.g. by attaching a charged leader to the peptide), the disclosed methods are readily applied to characterisation of unmodified peptides (although detection of peptides having leaders attached thereto is not excluded). Contrary to methods which rely on the use of motor proteins which may have variable ratchet step sizes to control the movement of a polypeptide with respect to a nanopore, the disclosed methods are simpler and allow the regular and predictable passage of a polypeptide through a nanopore. Contrary to methods which require a prior hypothesis about the position and/or nature of features that are subsequently sought to be identified during analysis (so-called “hypothesis-driven” methods), the disclosed methods do not require prior knowledge of the structure or characteristics of the peptide, polypeptide or protein to be characterised: features of the peptide, polypeptide or protein are detected during the real-time characterisation of the peptide, polypeptide or protein as it translocates through the nanopore. Other advantages of the disclosed methods will be apparent to those skilled in the art in view of the present disclosure, and are described herein.

Accordingly, in one aspect is provided herein a method of characterising a peptide, polypeptide or protein at least 25 amino acids in length; comprising
contacting the peptide, polypeptide or protein with an engineered protein nanopore having a first opening, a second opening and a solvent-accessible channel therebetween;
under conditions such that an electroosmotic force across the nanopore causes the peptide, polypeptide or protein to translocate through the nanopore in a linearised state; and
taking one or more measurements characteristic of the peptide, polypeptide or protein as the peptide, polypeptide or protein translocates the nanopore;
thereby characterising the peptide, polypeptide or protein.

In a related aspect, provided is a method of characterising one or more proteoforms of a peptide, polypeptide or protein; comprising
contacting the peptide, polypeptide or protein with a nanopore under conditions such that an electroosmotic force across the nanopore causes the peptide, polypeptide or protein to translocate through the nanopore in a linearised state; and
taking one or more measurements characteristic of the peptide, polypeptide or protein as the peptide, polypeptide or protein translocates the nanopore;
thereby characterising the proteoforms of the peptide, polypeptide or protein.

The above methods may be referred to herein as disclosed methods. For avoidance of doubt, herein embodiments of the present disclosure are described in relation to the disclosed methods for brevity. Unless required otherwise by the context, such embodiments are expressly disclosed in relation to and as preferred features of each of the disclosed methods above.

The disclosed methods are illustrated conceptually in non-limiting manner in FIG. 1.

The disclosed methods comprise taking one or more measurements characteristic of the peptide, polypeptide or protein as the peptide, polypeptide or protein moves with respect to a nanopore, e.g. as the peptide, polypeptide or protein translocates the nanopore. The one or more measurements can be any suitable measurements. Typically, the one or more measurements are electrical measurements, e.g. current measurements, and/or are one or more optical measurements. Apparatuses for recording suitable measurements, and the information that such measurements can provide, are described in more detail herein.

The measurements taken in the disclosed methods are typically characteristic of one or more characteristics of the peptide, polypeptide or protein, often selected from (i) the length of the polypeptide, (ii) the identity of the polypeptide, (iii) the sequence of the polypeptide, (iv) the secondary structure of the polypeptide, (v) whether or not the polypeptide is modified and (vi) the number, position(s) and/or location(s) of any modifications on the polypeptide. In typical embodiments the measurements are characteristic of the sequence of the peptide, polypeptide or protein or whether or not the peptide, polypeptide or protein is modified, e.g. by one or more post-translational modifications as described in more detail herein.

Suitable nanopores for use in the disclosed methods are also described in more detail herein. In some embodiments the nanopore is selected or modified to have be ion selective. In some embodiments the nanopore is modified to have an increased ion selectivity compared to the ion selectivity of the unmodified (reference) nanopore. In some embodiments the nanopore is modified to enhance or increase the electroosmotic force across the nanopore.

In some embodiments the methods are carried out under conditions that enhance the electroosmotic force experienced by the peptide, polypeptide or protein. In some embodiments the methods are carried out at a pH for promoting electroosmosis across the nanopore. However, those skilled in the art will appreciate that the disclosed methods are amenable to operation across a wide pH range according to the requirements of the user.

In some embodiments the methods are carried out in the presence of reaction components which may facilitate said methods. For example, in some embodiments the methods are carried out in the presence of a chaotropic agent. A chaoptropic agent may be a denaturant. In some embodiments the disclosed methods comprise contacting the peptide, polypeptide or protein with a chaotropic agent. Suitable agents are described in more detail herein. However, those skilled in the art will appreciate that there is no requirement for a chaotropic agent or denaturant to be present or used in the provided methods.

In some embodiments the peptide, polypeptide or protein is not attached to a charged leader. For example, in some embodiments the peptide, polypeptide or protein is not attached to a polynucleotide leader. In some embodiments the peptide, polypeptide or protein is not attached to an ionic polypeptide such as an anionic peptide. In some embodiments the peptide, polypeptide or protein is not attached to an anionic peptide such as a poly-aspartate, poly-glutamate or poly(aspartate/glutamate) leader. However, unless implied otherwise by the context, those skilled in the art will appreciate that in some embodiments a leader may be used in the disclosed methods.

In some embodiments the methods are carried out in the absence of a motor protein. Some known methods of characterising polypeptides using nanopores use motor proteins to control the movement of such polypeptides, but such motor proteins may sometimes be inefficient at precisely controlling the movement of long polypeptide strands, even though they may effectively translocate on such strands. In other words, in some embodiments a motor protein is not used to control the translocation of the peptide, polypeptide or protein through the nanopore. However, unless implied otherwise by the context, those skilled in the art will appreciate that in some embodiments a motor protein may be used in the disclosed methods.

In some embodiments, the methods involve characterising the polypeptide (e.g. proteoforms of the peptide, polypeptide or protein) by detecting the number, position and/or nature of modifications in said peptide, polypeptide or protein as the peptide, polypeptide or protein translocates through the nanopore. The characterisation may be real-time and in some embodiments does not require prior knowledge about the structure, sequence or properties of the peptide, polypeptide or protein.

### Characterising a Peptide, Polypeptide or Protein

Any suitable peptide, polypeptide or protein can be characterised using the methods disclosed herein. In some embodiments the peptide, polypeptide or protein is a protein or naturally occurring polypeptide. In some embodiments the peptide, polypeptide or protein is a complete intact peptide, polypeptide or protein. In some embodiments the peptide, polypeptide or protein is a portion of a protein or naturally occurring polypeptide, such as may be obtained by protease digestion of a protein or naturally occurring polypeptide. In some embodiments the polypeptide is a synthetic polypeptide. In some embodiments the peptide, polypeptide or protein is a conjugate of a plurality of polypeptides. In some embodiments, the peptide, polypeptide or protein is a concatamer of a plurality of polypeptides. Polypeptides which can be characterised in accordance with the disclosed methods are described in more detail herein.

In some embodiments the disclosed methods are methods of determining the amino acid sequence of said peptide, polypeptide or protein. In some embodiments the disclosed methods are for fingerprinting said peptide, polypeptide or protein. In some embodiments the disclosed methods are for detecting a tag or barcode of said peptide, polypeptide or protein. In some embodiments the disclosed methods are for determining the sequence of a tag or barcode of said peptide, polypeptide or protein. A tag or barcode may be a sequence of from about 5 to about 50, e.g. from about 10 to about 30 e.g. about 20 amino acids in length having a characteristic sequence or properties.

In some embodiments the disclosed methods are used for characterising one or more proteoforms of said peptide, polypeptide or protein.

As used herein, the term “proteoform” relates to different forms of peptide, polypeptide or proteins which may be produced with a variety of sequence variations, splice isoforms, and post-translational modifications. Proteoforms suitable for characterisation in accordance with the disclosed methods are described in Smith and Kelleher, Science 359 (6380) 1106-1107 (2018); and Smith and Kelleher, Nature Methods 10, 186-187 (2013); the entire contents of each are hereby incorporated by reference in their entirety. In some embodiments, proteoforms suitable for characterisation in accordance with the disclosed methods include proteoforms corresponding to modifications in the genome, modifications in the RNA, modifications during translation and modifications at the protein level. In some embodiments, proteoforms suitable for characterisation in accordance with the disclosed methods include somatic mutations, long-range genome rearrangements; recombinations (e.g. V (D) J recombinations), somatic hypermutations, alternative splicings, RNA base editing modifications, frameshift modifications, codon reassignments, translational bypass modifications, translational errors, modifications arising from proteolytic processing, protein splicing modifications, post-translational modifications (PTMs) and chemical rearrangements.

In some preferred embodiments the disclosed methods are methods of characterising one or more post-translational modifications in a peptide, polypeptide or protein. In some embodiments the disclosed methods are methods of detecting PTMs in a peptide, polypeptide or protein. In some embodiments the disclosed methods are methods of determining the presence, absence, number or position or one or more (e.g. two or more) PTMs in a peptide, polypeptide or protein. In some embodiments the disclosed methods are methods of determining the presence, absence, number or position of at least 2, 3, 4, 5, 6, 7, 8, 9, 10, 15, 20, 25, 30, 40, 50 or more PTMs in a peptide, polypeptide or protein. In some embodiments a peptide, polypeptide or protein is a concatamer as described in more detail herein. The disclosed methods can be used to characterise the extent to which a polypeptide has been post-translationally modified.

In some embodiments the disclosed methods are methods of determining the presence, absence, number or position or one or more (e.g. two or more) PTMs at one or more (e.g. two or more) sites within a peptide, polypeptide or protein. In some embodiments the disclosed methods are methods of determining the presence, absence, number or position or one or more PTMs at each of one or more (e.g. at least 2, 3, 4, 5, 6, 7, 8, 9, 10, 15, 20, 25, 30, 40, 50 or more) sites within a peptide, polypeptide or protein.

In some preferred embodiments the disclosed methods are methods of characterising one or more RNA splicing sites or modifications thereto in a peptide, polypeptide or protein. In some embodiments the disclosed methods are methods of detecting RNA splicing sites or modifications thereto in a peptide, polypeptide or protein. In some embodiments the disclosed methods are methods of determining the presence, absence, number or position or one or more (e.g. two or more) RNA splicing sites or modifications thereto in a peptide, polypeptide or protein. In some embodiments the disclosed methods are methods of determining the presence, absence, number or position of at least 2, 3, 4, 5, 6, 7, 8, 9, 10, 15, 20, 25, 30, 40, 50 or more RNA splicing sites or modifications thereto in a peptide, polypeptide or protein. In some embodiments a peptide, polypeptide or protein is a concatamer as described in more detail herein.

In some embodiments, said one or more sites are located at least 5, at least 10, at least 15, or at least 20 amino acids from the N-terminus of said peptide, polypeptide or protein. In some embodiments, said one or more sites are located at least 5, at least 10, at least 15, or at least 20 amino acids from the C-terminus of said peptide, polypeptide or protein. In some embodiments, said one or more sites are located at least 25 amino acids from the N-terminus and/or the C-terminus of said peptide, polypeptide or protein. In some embodiments, said one or more sites are located at least 30, at least 40, at least 50, at least 60, at least 70, at least 80, at least 90 or at least 100 amino acids from the N-terminus and/or the C-terminus of said peptide, polypeptide or protein. In some embodiments said one or more sites are buried within said protein. In some embodiments said one or more sites are not solvent-accessible. In some embodiments said one or more sites are not located at a solvent-accessible surface of said peptide, polypeptide or protein.

In some embodiments said one or more sites are separated in said peptide, polypeptide or protein by at least 10, at least 20, at least 30, at least 40, at least 50, at least 60, at least 70, at least 80, at least 90, at least 100, at least 150, at least 200, or more amino acids.

In some embodiments which involve detecting or determining the presence, absence, number or position(s) of one or more post-translational modifications in a peptide, polypeptide or protein, any one or more post-translational modifications may be present in the or each polypeptide. Typical post-translational modifications include modification with a hydrophobic group, modification with a cofactor, addition of a chemical group, glycation (the non-enzymatic attachment of a sugar), biotinylation and pegylation. Post-translational modifications can also be non-natural, such that they are chemical modifications (e.g. done in the laboratory) for biotechnological or biomedical purposes. This can allow monitoring the levels of the laboratory made peptide, polypeptide or protein in contrast to the natural counterparts.

Examples of post-translational modification with a hydrophobic group include myristoylation, attachment of myristate, a C₁₄ saturated acid; palmitoylation, attachment of palmitate, a C₁₆ saturated acid; isoprenylation or prenylation, the attachment of an isoprenoid group; farnesylation, the attachment of a farnesol group; geranylgeranylation, the attachment of a geranylgeraniol group; and glypiation, and glycosylphosphatidylinositol (GPI) anchor formation via an amide bond.

Examples of post-translational modification with a cofactor include lipoylation, attachment of a lipoate (C₈) functional group; flavination, attachment of a flavin moiety (e.g. flavin mononucleotide (FMN) or flavin adenine dinucleotide (FAD)); attachment of heme C, for instance via a thioether bond with cysteine; phosphopantetheinylation, the attachment of a 4′-phosphopantetheinyl group; and retinylidene Schiff base formation.

Examples of post-translational modification by addition of a chemical group include acylation, e.g. O-acylation (esters), N-acylation (amides) or S-acylation (thioesters); acetylation, the attachment of an acetyl group for instance to the N-terminus or to lysine; formylation; alkylation, the addition of an alkyl group, such as methyl or ethyl; methylation, the addition of a methyl group for instance to lysine or arginine; amidation; butyrylation; gamma-carboxylation; glycosylation, the enzymatic attachment of a glycosyl group for instance to arginine, asparagine, cysteine, hydroxylysine, serine, threonine, tyrosine or tryptophan; polysialylation, the attachment of polysialic acid; malonylation; hydroxylation; iodination; bromination; citrulination; nucleotide addition, the attachment of any nucleotide such as any of those discussed above, ADP ribosylation; oxidation; phosphorylation, the attachment of a phosphate group for instance to serine, threonine or tyrosine (O-linked) or histidine (N-linked); adenylylation, the attachment of an adenylyl moiety for instance to tyrosine (O-linked) or to histidine or lysine (N-linked); propionylation; pyroglutamate formation; S-glutathionylation; Sumoylation; S-nitrosylation; succinylation, the attachment of a succinyl group for instance to lysine; selenoylation, the incorporation of selenium; and ubiquitinilation, the addition of ubiquitin subunits (N-linked).

Preferred PTMs for detection by the disclosed methods are phosphorylations, glutathionylations and glycosylations, particularly phosphorylations.

As described in more detail herein, in some embodiments one or more labels can be used to promote the detection or characterisation (e.g. to detect or determine the presence, absence, identity, number or position(s)) of one or more PTMs in a peptide, polypeptide or protein.

### Linearised Translocation of Peptides, Polypeptides and Proteins

The disclosed methods comprise characterising a peptide, polypeptide or protein (or one or more proteoforms thereof) as the peptide, polypeptide or protein translocates through a nanopore in a linearised state.

As used herein, the term “linearised state” refers to a three-dimensional form of the peptide, polypeptide or protein in which secondary and/or tertiary structure is altered, typically decreased, relative to the native (folded) form of the peptide, polypeptide or protein. The term “linearised state” may be used synonymously with the term “unfolded state” as it is applied to peptides, polypeptides and proteins, unless implied otherwise by the context. As explained in more detail herein, a linearised state of a peptide, polypeptide or protein may be contrasted with a globular or folded state of the peptide, polypeptide or protein.

In general, peptides, polypeptides and proteins adopt globular folded forms on exposure to solvent (aqueous or non-aqueous) according to their sequence. For example, proteins are known to fold to adopt 3D structures which may be associated with their biological function.

Peptides, polypeptides and proteins typically adopt energetically favourable conformations arranged such that solvent-accessible amino acids are appropriate to the native environment of the protein (e.g. soluble proteins which may be released into aqueous cellular compartments or intracellular fluid typically have surface accessible amino acids having polar side chains, whereas membrane-anchored proteins may comprise surface-accessible non-polar amino acids).

For example, proteins may comprise structural motifs including alpha helixes, beta sheets, beta turns, omega loops, and the like. These motifs, also referred to as protein domains, are determined primarily by hydrogen bonding interactions between amino acids in the primary sequence of the peptide, polypeptide or protein, and determine the so-called secondary structure of the peptide, polypeptide or protein. The interaction of secondary-structural protein domains in three dimensional space determines the overall three-dimensional shape of the peptide, polypeptide or protein, which is referred to as its tertiary structure.

The presence of 3D structure (e.g. secondary or tertiary structure) in a target polypeptide may hamper its characterisation using a nanopore in known methods which rely on the electrophoretically-driven or enzymatically-driven translocation of peptides, polypeptides and proteins through the pore. This is because different portions of a folded target polypeptide will require different degrees of force to unfold them in order to translocate in this manner. The consequence of this is that the movement of the polypeptide through the pore can be irregular, for example with some portions moving more quickly through the pore compared to other portions. This can hamper accurate characterisation.

Some prior attempts to use electroosmotic force to detect long polypeptides have relied on detecting folded proteins in the globular state. However, these methods do not allow the polypeptides assessed therein to be accurately characterised, and also rely on the use of large nanopores which can accommodate such folded proteins. Such methods typically cannot be used to characterise proteoforms of peptides, polypeptides and proteins (such as internal PTMs). Improvements are needed.

In the disclosed methods, the translocation of the peptide, polypeptide or protein through the nanopore is typically translocation in a linearised (unfolded) state. In some embodiments the linearised state is a state where the tertiary structure of the native protein is decreased or removed. For example, in some embodiments the peptide, polypeptide or protein is devoid of at least 5%, at least 10%, at least 20%, at least 30%, at least 40%, at least 50%, at least 60%, at least 70%, at least 80%, at least 90%, at least 95%, at least 96%, at least 97%, at least 98%, or at least 99% of its native tertiary structure. In some embodiments the peptide, polypeptide or protein translocates the nanopore in a form devoid of its native tertiary structure.

In some embodiments the linearised state is a state where the secondary structure of the native protein is decreased or removed. For example, in some embodiments the peptide, polypeptide or protein is devoid of at least 5%, at least 10%, at least 20%, at least 30%, at least 40%, at least 50%, at least 60%, at least 70%, at least 80%, at least 90%, at least 95%, at least 96%, at least 97%, at least 98%, or at least 99% of its native secondary structure. In some embodiments the peptide, polypeptide or protein translocates the nanopore in a form devoid of its native secondary structure.

In some embodiments the linearized form is substantially devoid of secondary or tertiary structure. In some embodiments the linearized form is linear over at least 10, at last 15, at least 20, at least 30, at least 40, at least 50, at least 60, at least 70, at least 80, at least 90, at least 100, at least 150, at least 200, at least 300, at least 400, or at least 500 amino acids.

In some embodiments the linearised form is linear over the length of the nanopore. In some embodiments the linearised form is linear over the length of the channel running through the nanopore. In some embodiments the linearised form is linear over a length at least 2 times, 3 times, 4 times, 5 times, 6 times, 7 times, 8 times, 9 times, 10 times or more the length of the nanopore or channel therethrough.

The length of a polypeptide in a linearized form can be determined from the number of amino acids in the polypeptide if known, for example a peptide unit in a polypeptide is commonly considered to have a length of about 0.35 nm (3.5 Å). In some embodiments the unfolded form is linear over a length of at least 3, at least 4, at least 5, at least 6, at least 7, at least 8, at least 9 or at least 10 nm.

The polypeptide can be held in a linearized form using any suitable means.

In some embodiments the peptide, polypeptide or protein may be linearised (e.g. unfolded) by contacting the peptide, polypeptide or protein with a chemical agent. In some embodiments the chemical agent is a chaotropic agent. In some embodiments the chaotropic agent is a denaturant. In some embodiments the disclosed methods are conducted in the presence of a chaotropic agent such as a denaturant. In some embodiments the disclosed methods comprise contacting the peptide, polypeptide or protein with a chaotropic agent such as a denaturant prior to the translocation of the peptide, polypeptide or protein through the nanopore. Use of a chaotropic agent such as a denaturant is not essential to the disclosed methods, but is a specifically disclosed embodiment of the disclosed methods.

In some embodiments wherein a chaotropic agent such as a denaturant is used, the agent is selected from guanidinium salts (e.g. guanidine HCl), guanidinium isothiocyanate, urea and thiourea. Combinations of agents such as denaturants can be used. In some embodiments wherein a denaturant is used, the denaturant is a guanidinium salt (e.g. guanidine HCl).

In some embodiments wherein a chaotropic agent such as a denaturant is used, the agent is present at a concentration in the reaction medium of from about 10 mM to about 3 M, such as from about 100 mM to about 2 M, e.g. from about 250 mM to about 1.5 M, e.g. from about 500 mM to about 1 M such as from about 600 mM to about 800 mM, e.g. about 700 to 650 mM. If used, the concentration of such denaturants in the disclosed methods may be dependent on the peptide, polypeptide or protein to be characterised in the methods and can be readily selected by those of skill in the art. Typically, the chaotropic agent or denaturant does not disrupt the structure of the nanopore. In some embodiments a chaotropic agent is used at a concentration which does not disrupt the structure of the nanopore.

In other embodiments, the peptide, polypeptide or protein can be maintained in an unfolded (e.g. linearized) form by using suitable detergents. Suitable detergents for use in the disclosed methods include SDS (sodium dodecyl sulfate).

In other embodiments, the peptide, polypeptide or protein can be maintained in an unfolded (e.g. linearized) form by carrying out the disclosed methods at an elevated temperature. Increasing the temperature overcomes intra-strand bonding and allows the polypeptide to adopt a linearized form.

In some embodiments, a peptide, polypeptide or protein can be held in a linearized form by choosing an appropriate pH according to the peptide, polypeptide or protein to be characterised in the methods. Suitable pH values are described herein.

### Peptides, Polypeptides and Proteins

Any suitable polypeptide can be characterised in the disclosed methods.

In some embodiments the or each peptide, polypeptide or protein is an unmodified protein or a portion thereof. In some embodiments the or each peptide, polypeptide or protein is a naturally occurring polypeptide or a portion thereof. In some embodiments the or each peptide, polypeptide or protein is a complete intact protein.

In some embodiments the or each peptide, polypeptide or protein is secreted from cells. Alternatively, the or each peptide, polypeptide or protein can be produced inside cells such that it must be extracted from cells for characterisation by the disclosed methods. The or each peptide, polypeptide or protein may comprise the products of cellular expression of a plasmid, e.g. a plasmid used in cloning of proteins in accordance with the methods described in Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016).

In some embodiments the or each peptide, polypeptide or protein may be obtained from or extracted from any organism or microorganism. The or each polypeptide may be obtained from a human or animal, e.g. from urine, lymph, saliva, mucus, seminal fluid or amniotic fluid, or from whole blood, plasma or serum. The or each polypeptide may be obtained from a plant e.g. a cereal, legume, fruit or vegetable.

The or each peptide, polypeptide or protein can be provided as an impure mixture of one or more polypeptides and one or more impurities. Impurities may comprise truncated forms of the peptide, polypeptide or protein to be characterised. Impurities may also comprise peptides, polypeptides or proteins other than the peptide, polypeptide or protein to be characterised in the disclosed methods, e.g. which may be co-purified from a cell culture or obtained from a sample.

The or each peptide, polypeptide or protein may be labelled with a molecular label. A molecular label may be a modification to the polypeptide which promotes the detection of the polypeptide in the methods provided herein. For example the label may be a modification to the polypeptide which alters the signal obtained as conjugate is characterised. For example, the label may interfere with an electroosmotic flux of solvent molecules (e.g. water molecules) through the nanopore. In such a manner, the label may improve the sensitivity of the methods.

In some embodiments a label is a label for a characteristic feature of the peptide, polypeptide or protein to be characterised. In some embodiments the label is a label for a characteristic feature of the proteoform of the peptide, polypeptide or protein. For example, in some embodiments the label is a label for a post-translational modification.

The term “label” as used herein embraces moieties which may bind to the feature in order to promote characterisation of the feature in the provided methods. In some embodiments the label is a specific binder for the feature at issue. The term “label” and “binder” can be used interchangeably. The examples provided herein include examples of labels for detecting features of a peptide, polypeptide or protein such as post-translational modifications; an exemplary embodiment described herein includes the detection of phosphorylation in a peptide, polypeptide or protein but the invention is not limited to such embodiments. Those skilled in the art will be well aware that many binding moieties that can be used as labels in the methods provided herein can be used and in general it is straight forward to identify or produce a binding label for any feature of a peptide, polypeptide or protein of interest. In a general sense, the invention provides the use of a label or binder for a protein feature of interest, in order to promote characterisation of the feature using the methods disclosed herein.

In general a binder or label for use in the disclosed methods will generate a specific signal when it translocates through the nanopore in accordance with the methods provided herein. In some embodiments the binder or label augments the signal generated by the peptide, polypeptide or protein as the peptide, polypeptide or protein moves through the nanopore. In some embodiments the binder or label attenuates the signal generated by the peptide, polypeptide or protein as the peptide, polypeptide or protein moves through the nanopore. In some embodiments the binder or label changes one or more properties of the signal generated by the peptide, polypeptide or protein as the peptide, polypeptide or protein moves through the nanopore without changing the magnitude of the signal. For example, in some embodiments the binder or label alters the noise properties of the signal generated by the peptide, polypeptide or protein as the peptide, polypeptide or protein moves through the nanopore.

In some embodiments the binder or label has a steric bulk that impedes particle (e.g. water particle) flow through a nanopore and thus generates a blocking signal characteristic of the peptide, polypeptide or protein feature at issue when the label passes through the nanopore. Steric bulk can be provided by e.g. polymers (e.g. PEG groups) and large molecules such as large aromatic moieties (e.g. fused aromatic ring systems, macrocycles, etc). In some embodiments the binder or label has an optically active group such as a fluorophore that creates or alters (e.g. enhances) an optical signal when the characteristic of the peptide, polypeptide or protein feature at issue when the label passes through the nanopore. In some embodiments the binder or label has a chemically active group that binds (typically transiently, e.g. by hydrogen bonding or ionic interaction) with the nanopore when label passes through the nanopore.

Accordingly, in some embodiments the methods provided herein comprise labelling the peptide, polypeptide or protein with a molecular label characteristic of one or more features of the peptide, polypeptide or protein to be characterised, such as one or more post-translational modifications; and taking one or more measurements characteristic of the peptide, polypeptide or protein as the labelled peptide, polypeptide or protein translocates the nanopore. In some embodiments the methods further comprise detecting the presence, absence, number or position(s) of the molecular label during the translocation of the peptide, polypeptide or protein through the nanopore. In some embodiments the presence, absence, number or position(s) of the molecular label provides information on the presence, absence, number, position(s) or identity of post-translational modifications on the peptide, polypeptide or protein. For example, if the label is selective for a first type of PTM then a signal arising from the label during the translocation of the peptide, polypeptide or protein through the nanopore indicates that the first type of PTM is present.

Some exemplary binders include:
metal-based complexes (e.g. Phos-tag, Ga-IDA (IDA=Iminodiacetic Acid), Ni-NTA (NTA=nitrilotriacetic acid)) for labelling anionic PTMs (e.g. phosphorylation, sulfation);
boronic acids for labelling PTMs containing diols (e.g. glycosylation, ribosylation)
disulfide-reacting reagents (e.g. thiol-based reagents) for labelling disulfides or other redox PTMs (e.g. glutathionylation);
host molecules (e.g. cyclodextrins, calixarenes, bambusuril, cucurbituril etc) for labelling guest PTMs (e.g. lipidation);
nanobodies, antibodies, affibodies, minibodies (etc.) which are useful for labelling a wide variety of PTMs; and
proteins recognising specific epitopes such as deactivated enzymes: “dead” phosphotase, sulfatase, demethylase etc; “readers”: bromodomains, lectins etc.

Further example of binders or labels include: lectins, which may be used to label the glycosylation state of a peptide, polypeptide or protein; an aptamer (e.g., peptide aptamer, DNA aptamer, or RNA aptamer), an antibody, an anticalin, an ATP-dependent Clp protease adaptor protein (ClpS), an antibody binding fragment, an antibody mimetic, a peptide, a peptidomimetic, a protein, or a polynucleotide (e.g., DNA, RNA, peptide nucleic acid (PNA), a γPNA, bridged nucleic acid (BNA), xeno nucleic acid (XNA), glycerol nucleic acid (GNA), or threose nucleic acid (TNA), or a variant thereof).

Another strategy involves the azide labelling of PTMs, with the resulting azide-functionalised PTM being suitable for conjugation to a further detectable group.

It is within the abilities of those skilled in the art to provide a suitable binder for any PTM. For example, nanobodies can be generated to selectively label a desired PTM. In general, antibodies and antibody fragments can be produced to selectively label any desired amino acid sequence or fragment thereof and thus can be used in the methods provided herein.

In some embodiments the disclosed method comprises detecting the presence, absence, number or position(s) of one or more PTMs during the translocation of the peptide, polypeptide or protein through the nanopore. In some embodiments the one or more PTMs include one or more phosphorylations. In some embodiments the one or more phosphorylations are detected using a label or binder disclosed herein. In some embodiments the one or more phosphorylations are detected using a metal complex. In some embodiments the one or more phosphorylations are detected using a zinc-mediated “phos-tag” ligand.

In some embodiments a phos-tag ligand has a structure as shown below:

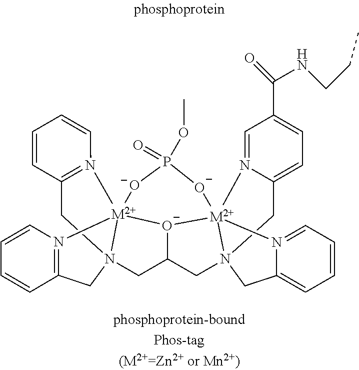

Accordingly, in some embodiments provided herein is a method of characterising one or more post-translational modifications in a peptide, polypeptide or protein; comprising
contacting the peptide, polypeptide or protein with a label capable of binding to said one or more post-translational modifications;
contacting the peptide, polypeptide or protein with a nanopore under conditions such that an electroosmotic force across the nanopore causes the peptide, polypeptide or protein to translocate through the nanopore in a linearised state; and
taking one or more measurements characteristic of the label as the peptide, polypeptide or protein translocates the nanopore;
thereby characterising the one or more post-translational modifications of the peptide, polypeptide or protein.

In some embodiments contacting the peptide, polypeptide or protein with a label capable of binding to said one or more post-translational modifications is conducted under conditions such that the label binds to said one or more post-translational modifications.

In some embodiments the one or more post-translational modification are any of the post-translational modifications disclosed herein, and the label is a selective label for said post-translational modification. In some embodiments the one or more post-translational modifications are one or more phosphorylations and the label comprises a metal complex. In some embodiments, therefore, provided is a method of characterising one or more phosphorylations in a peptide, polypeptide or protein; comprising
contacting the peptide, polypeptide or protein with a label capable of binding to said one or more phosphorylations under conditions such that the label binds to said one or more phosphorylations; wherein the label comprises a metal complex, such as a phos-tag ligand;
contacting the peptide, polypeptide or protein with a nanopore under conditions such that an electroosmotic force across the nanopore causes the peptide, polypeptide or protein to translocate through the nanopore in a linearised state; and
taking one or more measurements characteristic of the label as the peptide, polypeptide or protein translocates the nanopore;
thereby characterising the one or more phosphorylations of the peptide, polypeptide or protein.

In some embodiments the or each peptide, polypeptide or protein comprises sulphide-containing amino acids and thus has the potential to form disulphide bonds. Typically, in such embodiments, the polypeptide is reduced using a reagent such as DTT (Dithiothreitol) or TCEP (tris(2-carboxyethyl) phosphine) prior to being characterised using the disclosed methods.

A peptide, polypeptide or protein may comprise any combination of any amino acids, amino acid analogs and modified amino acids (i.e. amino acid derivatives). Amino acids (and derivatives, analogs etc) in the polypeptide can be distinguished by their physical size and charge. Amino acids/derivatives/analogs can be naturally occurring or artificial.

In some embodiments a peptide, polypeptide or protein may comprise any naturally occurring amino acid. Twenty amino acids are encoded by the universal genetic code. These are alanine (A), arginine (R), asparagine (N), aspartic acid (D), cysteine (C), glutamic acid/glutamate (E), glutamine (Q), glycine (G), histidine (H), isoleucine (I), leucine (L), lysine (K), methionine (M), phenylalanine (F), proline (P), serine(S), threonine (T), tryptophan (W), tyrosine (Y) and valine (V). Other naturally occurring amino acids include selenocysteine and pyrrolysine.

In some embodiments the or each polypeptide is a full length protein or naturally occurring polypeptide. In some embodiments a protein or naturally occurring polypeptide is fragmented prior to conjugation to the polynucleotide. In some embodiments the protein or polypeptide is chemically or enzymatically fragmented. In some embodiments polypeptides or polypeptide fragments can be conjugated to form a longer target polypeptide. In some embodiments a plurality of peptides, polypeptides or proteins may be concatamerized as described herein.

The or each peptide, polypeptide or protein can be a polypeptide of any suitable length. In some embodiments the peptide, polypeptide or protein is at least 20, at least 25, at least 30, at least 40, at least 50, at least 75, at least 100, at least 150, at least 200, or at least 500 peptide units (amino acids) in length. In some embodiments the or each polypeptide independently has a length of from about 25 to about 10,000 peptide units (amino acids). In some embodiments the polypeptide has a length of from about 50 or about 75 to about 7000 peptide units. In some embodiments the polypeptide has a length of from about 100 to about 5000 peptide units, for example from about 100 to about 2000 peptide units, e.g. from about 100 to about 1500 peptide units, such as from about 100 to about 1200 peptide units, e.g. from about 100 to about 1000 peptide units, e.g. from about 100 to about 500 peptide units such as from about 100 to about 250 peptide units.

In some embodiments the or each polypeptide independently has a length of from about 25 to about 10000 peptide units. In some embodiments the or each polypeptide independently has a length of from about 100 to about 5000 peptide units. In some embodiments the or each polypeptide has a length of from about 150 to about 2000 peptide units, for example from about 200 to about 1500 peptide units, e.g. from about 300 to about 1000 peptide units, such as from about 400 to about 700 peptide units, e.g. from about 450 to about 600 peptide units, e.g. about 500 peptide units.

Any number of polypeptides can be characterised in the disclosed methods. The peptides, polypeptides and proteins may be present in a sample comprising a plurality of peptides, polypeptides and/or proteins. For instance, the method may comprise characterising 2, 3, 4, 5, 6, 7, 8, 9, 10, 20, 30, 50, 100 or more polypeptides. The method may comprise characterising at least 10, at least 20, at least 50, at least 100, at least 500, at least 1000, at least 2000, at least 5000, at least 10000 or more peptides, polypeptides and proteins. If two or more polypeptides are used, they may be different polypeptides or two or more instances of the same polypeptide.

As explained herein, a leader is typically not present in the methods disclosed herein. However, in some embodiments where a leader may be present the leader is typically uncharged. For example, the leader may comprise a polymer such as PEG or a polysaccharide. The leader may be from 10 to 150 monomer units (e.g. ethylene glycol or saccharide units) in length, such as from 20 to 120, e.g. 30 to 100, for example 40 to 80 such as 50 to 70 monomer units (e.g. ethylene glycol or saccharide units) in length. However, it is not excluded that a charged leader can be used, such as a polynucleotide or charged polypeptide leader, when such leaders typically have a length of from 10 to 150 monomer units (e.g. nucleotide or amino acid units) in length, such as from 20 to 120, e.g. 30 to 100, for example 40 to 80 such as 50 to 70 monomer units (e.g. nucleotide or amino acid units) in length.

The or each peptide, polypeptide or protein typically has a low net charge. In some embodiments the peptide, polypeptide or protein has a net charge of between about −10 and about +10 per 50 amino acids; such as between about −5 and about +5 per 50 amino acids such as between about −3 and +3 per 50 amino acids.

In some embodiments the peptide, polypeptide or protein has a net charge of between about −5 and about +5 per 30 amino acids such as between about −3 and +3 per 30 amino acids e.g. between about −2 and about +2 per 30 amino acids.

In some embodiments the or each peptide, polypeptide or protein is substantially neutral, e.g. averaged across its length.

In some embodiments the peptide, polypeptide or protein is a concatamer. A concatamer, as used herein, is a construct comprising multiple copies of a peptide, polypeptide or protein attached together. In some embodiments the peptide, polypeptide or protein units in the concatamer are the same, i.e. the concatamer comprises multiple “repeat units” of a peptide, polypeptide or protein having a sequence to be characterised.

In some embodiments, a concatamer comprises at least 2, at least 3, at least 4, at least 5, at least 6, at least 7, at least 8, at least 9, at least 10, at least 20, at least 30, at least 40, at least 50, or at least 100 polypeptide portions. In some embodiments a concatamer as used herein may comprise from 2 to 50, such as from 3 to 25 e.g. from 4 to 20 such as from 5 to 15 e.g. from 8 to 12 repeat units.

The characterisation of concatamers may be useful in order to improve the accuracy of the characterisation data obtained. By forming concatamers of the peptide, polypeptide or protein to be characterised, multiple copies of the same amino acid sequence may be probed and data obtained accordingly. Such data may be compared (e.g. computationally processed) in order to obtain consensus data characteristic of the peptide, polypeptide or protein at issue.

Concatamers of peptides, polypeptides and proteins may be made in any suitable way.

In one embodiment, a concatamer may be produced by genetically encoding multiple copies of a peptide, polypeptide or protein of interest and expressing the concatamerized product. In other embodiments multiple peptide, polypeptide or proteins may be chemically or biochemically attached together into a single polymer chain. For example, in some embodiments the N-terminus of a peptide, polypeptide or protein may be chosen or modified in order to react with a C terminus of the peptide, polypeptide or protein, and appropriate conditions chosen or selected such that concatamers of desired length are produced. For example, by mixing peptide, polypeptide or protein units with reactive termini with equivalent peptide, polypeptide or protein units with inert termini concatamers of statistically definable length can be obtained, with the length determined by the ratio of reactive to non-reactive peptide, polypeptide or protein units present.

In some embodiments a concatamer may be obtained according to the methods described in the examples. In such methods the model protein thioredoxin (Trx) is used however those skilled in the art will appreciate that the disclosed methods are not specific to any particular protein and can be generally applied to any peptide, polypeptide or protein of interest.

In some embodiments a concatamer may be generated according to the methods described in Carrion-Vazquez et al, PNAS 96, 3694-3699) 1999), the entire contents of which are hereby incorporated by reference. In some embodiments a gene encoding a concatamer may be designed by amplifying a gene encoding the peptide, polypeptide or protein of interest into an expression vector. The gene may in some embodiments by present between restriction sites. Iterative cloning of monomer into monomer, dimer into dimer, tetramer into tetramer (etc) may be used in order to build up long concatamers.

In some embodiments, multiple peptide, polypeptide or protein units may be attached together to form a concatamer. For example, in some embodiments, a target peptide, polypeptide or protein may have a naturally occurring reactive functional group which can be used to facilitate conjugation to another peptide, polypeptide or protein. For example, cysteine residues can be used to form disulphide bonds.

In some embodiments a peptide, polypeptide or protein may be modified in order to facilitate its concatenation. For example, in some embodiments a peptide, polypeptide or protein may be modified by attaching a moiety comprising a reactive functional group for attaching to another peptide, polypeptide or protein unit.

For example, in some embodiments a peptide, polypeptide or protein may be extended at the N-terminus or the C-terminus by one or more residues (e.g. amino acid residues) comprising one or more reactive functional groups for reacting with a corresponding reactive functional group on another peptide, polypeptide or protein unit. For example, in some embodiments a polypeptide can be extended at the N-terminus and/or the C-terminus by one or more cysteine residues. Such residues can be used to build up a concatamer e.g. by maleimide chemistry (e.g. by reaction of cysteine with an azido-maleimide compound such as azido-[Pol]-maleimide wherein [Pol] is typically a short chain polymer such as a short chain PEG.

The chemistry used to build up concatamers from peptide, polypeptide or protein units is not particularly limited. Any suitable combination of reactive functional groups can be used. Many suitable reactive groups and their chemical targets are known in the art. Some exemplary reactive groups and their corresponding targets include aryl azides which may react with amine, carbodiimides which may react with amines and carboxyl groups, hydrazides which may react with carbohydrates, hydroxmethyl phosphines which may react with amines, imidoesters which may react with amines, isocyanates which may react with hydroxyl groups, carbonyls which may react with hydrazines, maleimides which may react with sulfhydryl groups, NHS-esters which may react with amines, PFP-esters which may react with amines, psoralens which may react with thymine, pyridyl disulfides which may react with sulfhydryl groups, vinyl sulfones which may react with sulfhydryl amines and hydroxyl groups, vinylsulfonamides, and the like.

Other suitable chemistry for conjugating a polypeptide to a polynucleotide includes click chemistry. Many suitable click chemistry reagents are known in the art. Suitable examples of click chemistry include, but are not limited to, the following:
(a) copper (I)-catalyzed azide-alkyne cycloadditions (azide alkyne Huisgen cycloadditions);
(b) strain-promoted azide-alkyne cycloadditions; including alkene and azide [3+2] cycloadditions; alkene and tetrazine inverse-demand Diels-Alder reactions; and alkene and tetrazole photoclick reactions;
(c) copper-free variant of the 1,3 dipolar cycloaddition reaction, where an azide reacts with an alkyne under strain, for example in a cyclooctane ring such as in bicycle [6.1.0]nonyne (BCN);
(d) the reaction of an oxygen nucleophile on one linker with an epoxide or aziridine reactive moiety on the other; and
(e) the Staudinger ligation, where the alkyne moiety can be replaced by an aryl phosphine, resulting in a specific reaction with the azide to give an amide bond.

Any reactive group(s) may be used to form the conjugate. Some suitable reactive groups include [1, 4-Bis [3-(2-pyridyldithio) propionamido]butane; 1,1 1-bis-maleimidotriethyleneglycol; 3,3′-dithiodipropionic acid di(N-hydroxysuccinimide ester); ethylene glycol-bis(succinic acid N-hydroxysuccinimide ester); 4,4′-diisothiocyanatostilbene-2,2′-disulfonic acid disodium salt; Bis [2-(4-azidosalicylamido)ethyl]disulphide; 3-(2-pyridyldithio) propionic acid N-hydroxysuccinimide ester; 4-maleimidobutyric acid N-hydroxysuccinimide ester; Iodoacetic acid N-hydroxysuccinimide ester; S-acetylthioglycolic acid N-hydroxysuccinimide ester; azide-PEG-maleimide; and alkyne-PEG-maleimide. The reactive group may be any of those disclosed in WO 2010/086602, particularly in Table 3 of that application.

In some embodiments the peptide, polypeptide or protein to be characterised in the disclosed methods may comprise a plurality of peptide, polypeptide or protein sections attached together by one or more linkers. The one or more linkers where present may be the same or different.

In some embodiments a linker comprises a polypeptide portion. For example, a plurality of proteins may be concatenated using a peptide linker which may be reacted with said proteins or may be genetically fused to said proteins such that it is expressed with the proteins. In some embodiments peptides, polypeptides and proteins for characterisation in the preferred methods are expressed as genetic fusion concatamers linked by genetically encoded peptide linkers as described herein. Such linkers can be readily introduced as described in the examples. Practitioners are also referred to methods disclosed in Sambrook et al., Molecular Cloning: A Laboratory Manual, 4^{th} ed., Cold Spring Harbor Press, Plainsview, New York (2012).

A linker may comprise or be an oligonucleotide (e.g., DNA, RNA, LNA, BNA, PNA, or morpholino). The oligonucleotide can have about 10-30 nucleotides in length or about 10-20 nucleotides in length. In some embodiments, the oligonucleotide can have at least one end (e.g., 3′- and/or 5′-end) modified for conjugation to the peptide, polypeptide or protein(s) to be characterised. The end modifiers may add a reactive functional group which can be used for conjugation. Examples of functional groups that can be added include, but are not limited to amino, carboxyl, thiol, maleimide, aminooxy, and any combinations thereof. Reagents for click chemistry (described herein) can also be used.

The linker may be a polymeric linker, such as polyethylene glycol (PEG), e.g. having a molecular weight of from about 500 Da to about 10 kDa, such as from about 1 kDa to about 5 kDa. The polymeric linker (e.g., PEG) can be functionalized with different functional groups including, e.g., but not limited to maleimide, NHS ester, dibenzocyclooctyne (DBCO), azide, biotin, amine, alkyne, aldehyde, and any combinations thereof.

In some embodiments, peptide linkers may be used. Preferred flexible peptide linkers comprise stretches of 2 to 50, such as about 10 to 40 e.g. about 20 to 30 amino acids. Serine, glycine and alanine are often used. A preferred linker comprising 29 amino acids (GSAGSAGSAGSAGSAGSAGSAGSAGSAGR; SEQ ID NO: 9) is described in the examples.

Linkers may be attached to peptides, polypeptides and proteins to be characterised using any methods known in the art. For example, a linker can be attached to a peptide, polypeptide or protein via one or more cysteines (cysteine linkage), one or more primary amines such as lysines, one or more non-natural amino acids, one or more histidines (His tags), etc. Such groups may be introduced to the peptide, polypeptide or protein(s) to be characterised by substitution. In some embodiments, peptides, polypeptides and proteins to be characterised may be chemically modified by attachment of (i) Maleimides including diabromomaleimides such as: 4-phenylazomaleinanil, 1.N-(2-Hydroxyethyl) maleimide, N-Cyclohexylmaleimide, 1.3-Maleimidopropionic Acid, 1.1-4-Aminophenyl-1H-pyrrole,2,5, dione, 1.1-4-Hydroxyphenyl-1H-pyrrole,2,5,dione, N-Ethylmaleimide, N-Methoxycarbonylmaleimide, N-tert-Butylmaleimide, N-(2-Aminoethyl) maleimide, 3-Maleimido-PROXYL, N-(4-Chlorophenyl) maleimide, 1-[4-(dimethylamino)-3,5-dinitrophenyl]-1H-pyrrole-2,5-dione, N-[4-(2-Benzimidazolyl)phenyl]maleimide, N-[4-(2-benzoxazolyl)phenyl]maleimide, N-(1-naphthyl)-maleimide, N-(2,4-xylyl) maleimide, N-(2,4-difluorophenyl) maleimide, N-(3-chloro-para-tolyl)-maleimide, 1-(2-amino-ethyl)-pyrrole-2,5-dione hydrochloride, 1-cyclopentyl-3-methyl-2,5-dihydro-1H-pyrrole-2,5-dione, 1-(3-aminopropyl)-2,5-dihydro-1H-pyrrole-2,5-dione hydrochloride, 3-methyl-1-[2-oxo-2-(piperazin-1-yl)ethyl]-2,5-dihydro-1H-pyrrole-2,5-dione hydrochloride, 1-benzyl-2,5-dihydro-1H-pyrrole-2,5-dione, 3-methyl-1-(3,3,3-trifluropropyl)-2,5-dihydro-1H-pyrrole-2,5-dione, 1-[4-(methylamino)cyclohexyl]-2,5-dihydro-1H-pyrrole-2,5-dione trifluroacetic acid, SMILES O═C1C═CC(═O)N1CC=2C═CN═CC2, SMILES O═C1C═CC(═O)N1CN2CCNCC2, 1-benzyl-3-methyl-2,5-dihydro-1H-pyrrole-2,5-dione, 1-(2-fluorophenyl)-3-methyl-2,5-dihydro 1H-pyrrole-2,5-dione, N-(4-phenoxyphenyl) maleimide, N-(4-nitrophenyl) maleimide (ii) Iodocetamides such as: 3-(2-Iodoacetamido)-proxyl, N-(cyclopropylmethyl)-2-iodoacetamide, 2-iodo-N-(2-phenylethyl) acetamide, 2-iodo-N-(2,2,2-trifluoroethyl) acetamide, N-(4-acetylphenyl)-2-iodoacetamide, N-(4-(aminosulfonyl)phenyl)-2-iodoacetamide, N-(1,3-benzothiazol-2-yl)-2-iodoacetamide, N-(2,6-diethylphenyl)-2-iodoacetamide, N-(2-benzoyl-4-chlorophenyl)-2-iodoacetamide, (iii) Bromoacetamides: such as N-(4-(acetylamino)phenyl)-2-bromoacetamide, N-(2-acetylphenyl)-2-bromoacetamide, 2-bromo-n-(2-cyanophenyl) acetamide, 2-bromo-N-(3-(trifluoromethyl)phenyl) acetamide, N-(2-benzoylphenyl)-2-bromoacetamide, 2-bromo-N-(4-fluorophenyl)-3-methylbutanamide, N-Benzyl-2-bromo-N-phenylpropionamide, N-(2-bromo-butyryl)-4-chloro-benzenesulfonamide, 2-Bromo-N-methyl-N-phenylacetamide, 2-bromo-N-phenethyl-acetamide,2-adamantan-1-yl-2-bromo-N-cyclohexyl-acetamide, 2-bromo-N-(2-methylphenyl) butanamide, Monobromoacetanilide, (iv) Disulphides such as: aldrithiol-2, aldrithiol-4, isopropyl disulfide, 1-(Isobutyldisulfanyl)-2-methylpropane, Dibenzyl disulfide, 4-aminophenyl disulfide, 3-(2-Pyridyldithio) propionic acid, 3-(2-Pyridyldithio) propionic acid hydrazide, 3-(2-Pyridyldithio) propionic acid N-succinimidyl ester, am6amPDP1-βCD and (v) Thiols such as: 4-Phenylthiazole-2-thiol, Purpald, 5,6,7,8-tetrahydro-quinazoline-2-thiol.

### Peptide, Polypeptide or Protein Movement

The direction of movement of the peptide, polypeptide or protein with respect to the nanopore is typically determined by the conditions under which the measurement is taken.

In some embodiments, the peptide, polypeptide or protein moves through the nanopore in a direction from the cis side of the nanopore to the trans side of the nanopore. In other embodiments, the peptide, polypeptide or protein moves through the nanopore in a direction from the trans side of the nanopore to the cis side of the nanopore.

In some embodiments it is advantageous that the peptide, polypeptide or protein moves multiple times through the nanopore. Any suitable method can be used to achieve this.

For example, an electrophoretic force counter to the electroosmotic force may be used. In such embodiments, the peptide, polypeptide or protein moves with respect to the nanopore under the electroosmotic force in accordance with the disclosed methods and is thereby characterised. An electrophoretic or mechanical force counter to the electroosmotic force may then be applied to bias the movement of the peptide, polypeptide or protein through the nanopore opposite to the electroosmotic force. The electrophoretic or mechanical force may then be reduced or halted and the peptide, polypeptide or protein may be re-characterised under the electroosmotic force in accordance with the disclosed methods.

The movement of the peptide, polypeptide or protein through the nanopore multiple times allows the accuracy of the characterisation of the peptide, polypeptide or protein to be improved.

Accordingly, in some embodiments the methods comprise:
i) carrying out a method described herein such that the peptide, polypeptide or protein translocates the nanopore in a first direction with respect to the nanopore;
ii) allowing the peptide, polypeptide or protein to move in a direction opposite to the direction of movement with respect to the nanopore in step (i) such that the peptide, polypeptide or protein translocates the nanopore in a second direction which is opposite to the first direction;
iii) optionally repeating steps (i) and (ii) to oscillate the polypeptide through the nanopore.

In such embodiments, steps (i) and (ii) may be repeated any number of times in order to obtain data of the required accuracy. For example, steps (i) and (ii) may be repeated at least 2 times, at least 3 times, at least 4 times, at least 5 times, at least 10 times, at least 20 times, at least 50 times, at least 100 times, at least 500 times or more.

In the disclosed methods, the movement of the peptide, polypeptide or protein through the nanopore is driven by electroosmotic force as described herein.

The electroosmotic force may be determined, chosen or enhanced according to the requirements of the user using any means known in the art.

For example, the electroosmotic force may be increased by reducing the pH. At low pH (e.g. from about pH 2 to about pH 5) basic amino acid side chains in the channel of the nanopore may be protonated and thus have a higher charge. At high pH (e.g. from about pH 8 to about pH 10) acidic amino acid side chains in the channel of the nanopore may be deprotonated and thus have a higher charge. The use of low pH to increase electroosmotic force on a very short polypeptide translocating through a nanopore has been demonstrated. However, the translocation of long polypeptides or characterisation thereof has not been demonstrated.

Modifications to increase the charge of the channel through the nanopore may be made in other ways. For example, chemical modification of solid state nanopores can be used to functionalise the substrate material in order to increase its charge. Protein nanopores can be modified e.g. by mutation to insert charged amino acids into the channel therethrough in order to increase the electroosmotic force through the nanopore. This is described in more detail herein.

In some embodiments the movement of the peptide, polypeptide or protein may be modulated by a physical or chemical force (potential). In some embodiments the physical force is provided by an electrical (e.g. voltage) potential or a temperature gradient, etc. In some embodiments the chemical force is provided by a concentration (e.g. pH) gradient.

In some embodiments, the movement of the peptide, polypeptide or protein is modulated by mechanically manipulating the peptide, polypeptide or protein thereby moving said construct, polynucleotide-polypeptide conjugate strand and/or polynucleotide carrier strand with respect to the nanopore.

In some embodiments the electroosmotically-driven translocation of polypeptides across a nanopore has an electrophoretic component. For example, if the polypeptide has a high net charge then a electrophoretic force may apply to the polypeptide. However, the inventors have proven herein that electroosmotic force can be used to translocate a peptide, polypeptide or protein through a nanopore in order to facilitate its characterisation under conditions inconsistent with electrophoretic translocation through the pore. Thus, in some embodiments, the electroosmotic force exceeds any electrophoretic component of the force acting on the peptide, polypeptide or protein. In some embodiments the electroosmotic force exceeds any electrophoretic component of the force acting on the peptide, polypeptide or protein by at least 2 times, at least 3 times, at least 4 times, at least 5 times, at least 10 times, at least 20 times, at least 30 times, at least 40 times, at least 50 times, at least 100 times or at least 1000 times.

In some embodiments the movement of the peptide, polypeptide or protein is modulated using a method as described in WO 2020/016573, the entire contents of which are incorporated herein by reference.

In some embodiments, the movement of the peptide, polypeptide or protein is modulated by applying a voltage to the peptide, polypeptide or protein. In some embodiments the applied voltage varies during the method. In some embodiments the applied voltage is a voltage ramp. A voltage ramp may be a regular or irregular change in the applied voltage between about −2 V to about +2 V and/or vice versa. More typically the voltage ramp is a ramp between about −400 mV and +400 mV, such as between about-300 mV and +300 mV, e.g. between about −200 mV and +200 mV, such as between about-100 mV and +100 mV. The voltage ramp may be between a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. For example, a voltage ramp may be from about 0 m V to about +100, +200, +300 or +400 mV, or from about 0 mV to about −100, −200, −300 or −400 mV. Other voltages may be applied and selection of appropriate voltages is within the capacity of the skilled person. Applying a variable voltage during the disclosed method can be advantageous in permitting peptides, polypeptides and proteins in a heterogeneous sample (or an ostensibly homogeneous sample, but wherein there is natural or induced variation in the peptides, polypeptides and proteins in the sample) to be probed.

As explained herein, the methods of the present disclosure are typically enzyme-free. However, in some embodiments (unless implied otherwise by the context) a motor protein may be used to control the translocation of the peptide, polypeptide or protein through the nanopore. Suitable motor proteins (also known as polynucleotide handling enzymes) include proteins of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31, such as helicases, polymerases, exonucleases, topoisomerases, and variants thereof. Suitable enzymes include exonuclease I or II from E. coli, RecJ from T. thermophiles, bacteriophage lambda exonuclease, TatD exonuclease, PyroPhage® 3173 DNA Polymerase (commercially available from Lucigen® Corporation), SD Polymerase (commercially available from Bioron®), Klenow (from NEB), Phi29 DNA polymerase, and helicases such as Hel308, RecD, TraI, TrwC, XPD, Dda, NS3, UvrD, Rep, PcrA, Pif1 and TraI. If used, a motor protein may be chosen or modified to prevent it from disengaging from the peptide, polypeptide or protein other than by passing off the end of the peptide, polypeptide or protein, for example as disclosed in WO 2014/013260.

If used, a motor protein may be operated in either an active or passive mode. In an active mode (e.g. when provided with all the necessary components to facilitate movement, such as fuel molecules (e.g. nucleotides such as adenosine triphosphate (ATP) and cofactors (e.g. divalent metal cations such as Mg²⁺) the motor protein may move along the polynucleotide in a 5′ to 3′ or a 3′ to 5′ direction (depending on the motor protein). The motor protein can be used to either move the peptide, polypeptide or protein away from (e.g. out of) the pore (e.g. against an electroosmotic force) or towards (e.g. into) the pore (e.g. with an electroosmotic force). In a passive (inactive mode) (e.g. when not provided with the necessary components to facilitate movement) the motor protein may bind to the peptide, polypeptide or protein and act as a brake slowing the movement of the peptide, polypeptide or protein with respect to the nanopore.

### Nanopore

As explained above, the disclosed methods comprise characterising a peptide, polypeptide or protein (or one or more proteoforms thereof) as the peptide, polypeptide or protein moves through a nanopore under an electroosmotic force.

Any suitable nanopore can be used. In one embodiment a nanopore is a transmembrane pore.

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

A transmembrane pore suitable for use in the invention may be a solid state pore. A solid-state nanopore is typically a nanometer-sized hole formed in a synthetic membrane. Suitable solid state pores include, but are not limited to, silicon nitride pores, silicon dioxide pores and graphene pores. Solid state nanopores may be fabricated e.g. by focused ion or electron beams, so the size of the pore can be tuned freely. Suitable solid state pores and methods of producing them are discussed in U.S. Pat. No. 6,464,842, WO 03/003446, WO 2005/061373, U.S. Pat. Nos. 7,258,838, 7,466,069, 7,468,271 and 7,253,434, each of which is incorporated by reference in their entirety.

A transmembrane pore may be a DNA origami pore as disclosed in Langecker et al., Science, 2012; 338:932-936 and in WO 2013/083983, each of which is incorporated by reference in their entirety. A transmembrane pore may be a scaffold based pore, such as a DNA-scaffold protein nanopore as disclosed in E. Spruijt, Nat. Nanotechnol. 2018, incorporated by reference.

A transmembrane pore may be a polymer-based pore. Suitable pores can be made from polymer-based plastics such as a polyester e.g. polyethylene terephthalate (PET) via track etching.

A transmembrane pore suitable for use in the invention may be a transmembrane protein pore. A transmembrane protein pore is a polypeptide or a collection of polypeptides that permits ions driven by an applied potential to flow from one side of a membrane to the other side of the membrane. Transmembrane protein pores are particularly suitable for use in the invention.

A transmembrane protein pore may be isolated, substantially isolated, purified or substantially purified. A pore is isolated or purified if it is completely free of any other components, such as lipids or other pores. A pore is substantially isolated if it is mixed with carriers or diluents which will not interfere with its intended use. For instance, a pore is substantially isolated or substantially purified if it present in a form that comprises less than 10%, less than 5%, less than 2% or less than 1% of other components, such as lipids or other pores. The pore is typically present in a membrane, for example a lipid bilayer or a synthetic membrane e.g. a block-copolymer membrane.

A transmembrane protein pore may be a monomer or an oligomer. A transmembrane protein pore is often made up of several repeating subunits, such as at least 6, at least 7, at least 8, at least 9, at least 10, at least 11, at least 12, at least 13, at least 14, at least 15, or at least 16 subunits. The pore is typically a hexameric, heptameric, octameric or nonameric pore.

The pore may be a homo-oligomer or a hetero-oligomer. A transmembrane protein pore may be a heptameric pore.

A transmembrane protein pore may typically comprises a barrel or channel through which the ions may flow. The subunits of the pore typically surround a central axis and contribute strands to a transmembrane β barrel or channel or a transmembrane α-helix bundle or channel.

Suitable transmembrane pores for use in accordance with the invention can be β-barrel pores, a-helix bundle pores or solid state pores. B-barrel pores comprise a barrel or channel that is formed from β-strands. Suitable B-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from a-helices. Suitable α-helix bundle pores include, but are not limited to, inner membrane proteins and a outer membrane proteins, such as Wza (e.g. see K. R. Mahendran, Nat. Chem. 2016, incorporated by reference) and ClyA toxin. For example, the transmembrane pore may be derived from or based on Msp, α-hemolysin (α-HL), lysenin, Phi29, CsgG, CgsF, ClyA, Sp1 and haemolytic protein fragaceatoxin C (FraC).

For example, the pore may be derived from α-hemolysin (α-HL). The wild type α-HL pore is formed of seven identical monomers or subunits (i.e. it is heptameric). The sequence of one wild type monomer or subunit of α-hemolysin is shown in SEQ ID NO: 1. Amino acids 1, 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228, 236 to 242, 262 to 265, 272 to 274, 287 to 290 and 293 of SEQ ID NO: 1 form loop regions. Residues 111, 113 and 147 of SEQ ID NO: 1 form part of a constriction of the barrel or channel of α-HL.

As will be apparent from the above discussion, nanopores for use in the disclosed methods typically have a first opening, a second opening and a solvent-accessible channel therebetween.

In some embodiments the solvent-accessible channel is modified in order to promote or increase electroosmotic flow through the nanopore in the disclosed methods. A modified protein nanopore may be referred to as an engineered protein nanopore. An engineered protein nanopore may be a mutated protein nanopore. Examples of mutations that can be made in protein nanopores are described in more detail herein. An engineered protein nanopore may be modified (e.g. by covalent or non-covalent modification). An engineered protein nanopore may be a synthetic nanopore. A synthetic nanopore may be assembled, e.g. by native chemical ligation.

In some embodiments wherein the nanopore is a protein nanopore, the channel comprises one or more non-native charged amino acids. The one or more non-native charged amino acids may for example be preferably located near a constriction of the barrel or channel. The one or more non-native charged amino acids may increase the electroosmotic flow through nanopore. The term “non-native” in this context refers to an amino acids which is not present at the relevant position in the wild-type pore; for example, as the result of a point mutation. “Non-native” amino acids may be canonical amino acids or non-canonical (e.g. artificial or modified) amino acids.

In some embodiments, the one or more non-native charged moieties increase the ion selectivity of the nanopore. In some embodiments, the one or more non-native charged moieties increase the ion selectivity of the nanopore by at least 10%, such as at least 50%, at least 80%, at least 90%, at least 100%, at least 150%, at least 200%, at least 500%, at least 1000% or more. In some embodiments, the one or more non-native charged moieties increase the anion selectivity of the nanopore. In some embodiments, the one or more non-native charged moieties increase the anion selectivity of the nanopore by at least 10%, such as at least 50%, at least 80%, at least 90%, at least 100%, at least 150%, at least 200%, at least 500%, at least 1000% or more. In some embodiments the anion selectivity is defined as P_{Na+}/P_{Cl-}<1. In some embodiments P_{Na+}/P_{Cl-} is less than 0.8, e.g. less than 0.6, e.g. less than 0.5, e.g. less than 0.4, e.g. less than 0.3, e.g. less than 0.2, e.g. less than 0.1.

In some embodiments, the one or more non-native charged moieties increase the cation selectivity of the nanopore. In some embodiments, the one or more non-native charged moieties increase the cation selectivity of the nanopore by at least 10%, such as at least 50%, at least 80%, at least 90%, at least 100%, at least 150%, at least 200%, at least 500%, at least 1000% or more. In some embodiments the cation selectivity is defined as P_{Cl-}/P_{Na+}<1. In some embodiments P_{Cl-}/P_{Na+} is less than 0.8, e.g. less than 0.6, e.g. less than 0.5, e.g. less than 0.4, e.g. less than 0.3, e.g. less than 0.2, e.g. less than 0.1.

In some embodiments the one or more non-native charged amino acids are positively charged amino acids, such as arginine, lysine or histidine. In some embodiments the one or more non-native charged moieties comprise one or more positively charged amino acids and said one or more positively charged amino acids increase the anion selectivity of the nanopore.

In some embodiments the one or more non-native charged amino acids are negatively charged amino acids, such as glutamatic acid (glutamate) or aspartic acid (aspartate). In some embodiments the one or more non-native charged moieties comprise one or more negatively charged amino acids and said one or more negatively charged amino acids increase the cation selectivity of the nanopore.

Other polar amino acids that can be incorporated to increase the charge of the channel are set out in Table 1 above.

Useful mutations to increase positive charge in the channel running through the nanopore include E→N (e.g. at a position corresponding to position 111 of SEQ ID NO: 1); M→R or K (e.g. at a position corresponding to position 113 of SEQ ID NO: 1); D→R; E→K, etc. Useful mutations to increase negative charge in the channel running through the nanopore include N→E (e.g. at a position corresponding to position 111 of SEQ ID NO: 1); M→D or E (e.g. at a position corresponding to position 113 of SEQ ID NO: 1); R→D; K→E, etc.

The one or more non-native charged amino acids may be one or more non-natural amino acids. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444. Charged non natural amino acids also include Trans-ACBD (CAS 73550-55-7); (2S,4R)-4-(carboxymethyl) pyrrolidine-2-carboxylic acid; piperidine-2,4-dicarboxylic acid; 2,6-diaminohex-4-ynoic acid; 1,4-diaminocyclohexane-1-carboxylic acid; 2-amino-3-(1H-imidazol-1-yl) propanoic acid, all available from Enamine.

In some embodiments the solvent-accessible channel comprises at least 1, at least 2, at least 3, at least 4, at least 5, at least 6, at least 7, at least 8, at least 9, at least 10 or more non-native charged amino acids. In some embodiments each monomer of a protein nanopore comprises at least 1, at least 2, at least 3, at least 4, at least 5, at least 6, at least 7, at least 8, at least 9, at least 10 or more non-native charged amino acids and at least 1, at least 2, at least 3, at least 4, at least 5, at least 6, at least 7, at least 8, at least 9, at least 10 or more non-native charged amino acids are at residues in the monomer such that they are in the solvent-accessible channel of the nanopore when the monomer oligomerises to form a nanopore.

In some embodiments the one or more non-native charged amino acids include a non-native amino acid at a position corresponding to position 113 in SEQ ID NO 1. In some embodiments the non-native charged amino acids include a positively charged amino acid residue (e.g. an arginine) at a position corresponding to position 113 in SEQ ID NO 1. In some embodiments at least 1, at least 2, at least 3, at least 4, at least 5, at least 6, or at least 7 monomers in the protein nanopore have a positively charged amino acid residue (e.g. an arginine) at a position corresponding to position 113 in SEQ ID NO 1. In some embodiments the nanopore is a homooligomeric nanopore and all of the monomers of the nanopore comprise a positively charged amino acid residue (e.g. an arginine) at a position corresponding to position 113 in SEQ ID NO 1. In some embodiments the nanopore is a heterooligomeric nanopore and at least one monomer of the nanopore comprises a positively charged amino acid residue (e.g. an arginine) at a position corresponding to position 113 in SEQ ID NO 1.

Other mutations may also be made. For example, in some embodiments the nanopore comprises asparagine at the position corresponding to position 111 in SEQ ID NO: 1 and/or asparagine at the position corresponding to position 147 in SEQ ID NO: 1.

The amino acid sequence of the exemplary NN-113R variant of SEQ ID NO: 1 as used in the examples is provided in SEQ ID NO: 2. Other protein nanopores may comprise equivalent modifications at positions corresponding to the modified positions of SEQ ID NO: 2 compared to SEQ ID NO: 1.

### Membrane

Typically, in the disclosed methods, the nanopore is typically present in a membrane. Any suitable membrane may be used in the system.

Suitable membranes are well-known in the art. The membrane is typically an amphiphilic layer. An amphiphilic layer is a layer formed from amphiphilic molecules, such as phospholipids, which have both at least one hydrophilic portion and at least one lipophilic or hydrophobic portion. The amphiphilic layer may be a monolayer or a bilayer. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450).

In some embodiments the membrane comprises one or more archaebacterial bipolar tetraether lipids or mimcs thereof. Such lipids are generally found in extremophiles such as that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes.

Block copolymers are polymeric materials in which two or more monomer sub-units polymerized together create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphipiles. The copolymer may be a triblock, tetrablock or pentablock copolymer. Typically the copolymer is a triblock copolymer comprising two monomer subunits A and B in an A-B-A pattern; typically the A monomer subunit is hydrophilic and the B subunit is hydrophobic.

The amphiphilic layer is typically a planar lipid bilayer or a supported bilayer.

The amphiphilic layer is typically a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is usually a planar lipid bilayer. Suitable lipid bilayers are disclosed in WO 2008/102121, WO 2009/077734 and WO 2006/100484).

Any lipid composition that forms a lipid bilayer may be used. Lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy (Polyethylene glycol)-2000]; functionalised PEG Lipids, such as 1,2-Distearoyl-sn-Glycero-3 Phosphoethanolamine-N-[Biotinyl(Polyethylene Glycol) 2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

Other components that affect the properties of the amphiphilic layer may be incorporated, such as fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

Methods for forming lipid bilayers are known in the art. Suitable methods are disclosed in the Example. Lipid bilayers are commonly formed by the method of Montal and Mueller (Proc. Natl. Acad. Sci. USA., 1972; 69:3561-3566), in which a lipid monolayer is carried on aqueous solution/air interface past either side of an aperture which is perpendicular to that interface. The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers. The lipid bilayer may be formed as described in WO 2009/077734. A lipid bilayer may also be a droplet interface bilayer formed between two or more aqueous droplets each comprising a lipid shell such that when the droplets are contacted a lipid bilayer is formed at the interface of the droplets.

In another preferred embodiment, the membrane is a solid state layer. A solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from monatomic layers, such as graphene, or layers that are only a few atoms thick. Suitable graphene layers are disclosed in WO 2009/035647. The nanopore may in some embodiments be present in an amphiphilic membrane or layer contained within the solid state layer, for instance within a hole, well, gap, channel, trench or slit within the solid state layer. Suitable systems are disclosed in WO 2009/020682 and WO 2012/005857. Any of the amphiphilic membranes or layers discussed above may be used.

### Conditions

Any suitable apparatus can be used to enact the methods of the present disclosure.

Electrical measurements may be made using standard single channel recording equipment as describe in Stoddart, D. S., et al., (2009), Proceedings of the National Academy of Sciences of the United States of America 106, p 7702-7707, Lieberman K R et al, J Am Chem Soc. 2010; 132 (50): 17961-72, and International Application WO 2000/28312, each of which is incorporated by reference in its entirety. Alternatively, electrical measurements may be made using a multi-channel system, for example as described in International Application WO 2009/077734 and International Application WO 2011/067559, each of which is incorporated by reference in its entirety.

In some embodiments the disclosed methods are carried out using an apparatus that is suitable for investigating a membrane/pore system in which a pore is inserted into a membrane. The disclosed methods may be carried out using any apparatus that is suitable for transmembrane pore sensing. For example, the apparatus may comprise a chamber comprising an aqueous solution and a barrier that separates the chamber into two sections. The barrier may have an aperture in which the membrane containing the pore is formed. The disclosed methods may also be carried out using droplet interface bilayers (DIBs). Two water droplets may be placed on electrodes and immersed into a oil/phospholipid mixture. The two droplets may be taken in close contact and at the interface a phospholipid membrane may be formed where the pores get inserted.

The disclosed methods may be carried out using the apparatus described in International Application WO 2008/102120.

The disclosed methods typically involve measuring the current flowing through a pore. Therefore the apparatus may also comprise an electrical circuit capable of applying a potential and measuring an electrical signal across a membrane and pore. The methods may be carried out using a patch clamp or a voltage clamp. The methods usually involve the use of a voltage clamp.

The characterisation methods may comprise optical measurements, for example such as described in WO 2016/009180 and WO 2021/198695.

The methods may be carried out on a silicon-based array of wells where each array comprises 128, 256, 512, 1024 or more wells, such as 2000, 3000, 4000, 6000, 10000, 12000, 15000 or more wells.

The methods may be carried out using an array of nanopores as described herein. The use of an array of pores may allow the monitoring of the method by monitoring a signal such an electrical or optical signal. The optical detection of analytes using an array of nanopores can be conducted using techniques known in the art, such as those described by Huang et al, Nature Nanotechnology (2015) 10: 986-992

The methods of the invention may involve the measuring of a current flowing through a pore. Suitable conditions for measuring ionic currents through transmembrane pores are known in the art and disclosed in the Example. The method is typically carried out with a voltage applied across the membrane and pore. The voltage used is typically from +2 V to −2 V, typically −400 mV to +400 mV. The voltage used is typically in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more often in the range 100 mV to 240 mV and most usually in the range of 120 mV to 220 mV.

The methods of the invention may be carried out in the presence of charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is typically from 150 mM to 1 M. The method is usually carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. The salt concentration used on each side of the membrane may be different, such as 0.1 M at one side and 3 M at the other. The salt and composition used on each side of the membrane may be also different. The use of asymmetric charge conditions can maximise the electroosmotic force through the nanopore.

The methods are typically carried out in the presence of a buffer. In the exemplary apparatus discussed above, the buffer is present in the aqueous solution in the chamber. Any buffer may be used in the method of the invention. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is typically about 7.5. In some embodiments the disclosed methods are conducted between about pH 4 and about pH 10. In some embodiments the disclosed methods are conducted between about pH 5 and about pH 9. In some embodiments the disclosed methods are conducted between about pH 6 and about pH 8. In some embodiments the disclosed methods are conducted about pH 7, such as about pH 7.2.

A reducing agent such as TCEP (tris(2-carboxyethyl) phosphine) may be present, e.g. at a concentration of from about 1 mM to about 50 mM such as from about 5 mM to about 20 mM, e.g. about 10 mM.

The methods may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature.

### System

Also provided is a system, comprising
an engineered protein nanopore having a first opening, a second opening and a solvent-accessible channel therebetween;
and
a peptide, polypeptide or protein at least 25 amino acid in length;
wherein said nanopore and/or said peptide, polypeptide or protein is present in a medium comprising a chaotropic agent.

In some embodiments, the system is configured such that when the peptide, polypeptide or protein is contacted with the nanopore an electroosmotic force across the nanopore is capable of causing the peptide, polypeptide or protein to translocate through the nanopore in a linearised state.

In some embodiments, the nanopore is comprised in a membrane and said system further comprises means for detecting electrical and/or optical signals across said membrane.

In some embodiments, the peptide, polypeptide or protein comprises one or more post-translational modifications and/or one or more RNA splicing sites.

In some embodiments the nanopore; peptide, polypeptide or protein; reaction medium; denaturant; membrane and means for detecting electrical or optical signals across said membrane are as described in more detail herein.

In some embodiments the system comprises a label for selectively binding to one or more post-translational modifications comprised in the peptide, polypeptide or protein.

The system may be configured for use with an algorithm, also provided herein, adapted to be run on a computer system. The algorithm may be adapted to detect information characteristic of a peptide, polypeptide or protein (e.g. characteristic of the sequence of the peptide, polypeptide or protein and/or whether the peptide, polypeptide or protein is modified), and to selectively process the signal obtained as the peptide, polypeptide or protein moves with respect to the nanopore.

In some embodiments a system comprises computing means configured to detect information characteristic of a peptide, polypeptide or protein (e.g. characteristic of the sequence of the peptide, polypeptide or protein and/or whether the peptide, polypeptide or protein is modified) and to selectively process the signal obtained as a peptide, polypeptide or protein translocates the nanopore. In some embodiments the system comprises receiving means for receiving data from detection of the peptide, polypeptide or protein, processing means for processing the signal obtained as the peptide, polypeptide or protein with respect to the nanopore, and output means for outputting the characterisation information thus obtained.

It is to be understood that although particular embodiments, specific configurations as well as materials and/or molecules, have been discussed herein for methods according to the present invention, various changes or modifications in form and detail may be made without departing from the scope and spirit of this invention. The preceding embodiments and following examples are provided for illustration only, and should not be considered limiting the application. The application is limited only by the claims.

### EXAMPLES

### Example 1

Means to sequence DNA and RNA quickly and cheaply have revolutionized biology and medicine¹. The ability to analyse cellular proteins and their millions of variants would be an advance of comparable importance, but requires a fresh technical approach². Here, we use electroosmosis for the non-enzymatic capture, unfolding and translocation of individual polypeptides of more than 1200 residues by a protein nanopore. By monitoring the ionic current carried by the nanopore, we locate post-translational modifications deep within the polypeptide chains, and thereby lay the groundwork for obtaining inventories of the proteoforms in cells and tissues.

The example describes the electroosmotically driven translocation of thioredoxin (Trx) concatamers through a mutant aHL nanopore. However, those skilled in the art will appreciate that the disclosed methods are not limited thereto. In particular, they are amenable to a wide variety of nanopores and peptide, polypeptide or protein analytes.

### Context

Single-molecule nanopore proteomics is gaining momentum². Nanopore sequencing of ultralong DNA and RNA has enabled biomedical applications that challenge short-read technologies. Modulation of the ionic current passing through a nanopore might also be used to distinguish and count the millions of proteoforms expressed from the 20,000 or so protein-encoding human genes. In this way, inventories would be obtained of variations such as post-translational modifications (PTMs) and alternative RNA splicing, which are often present at multiple locations throughout a polypeptide chain³. While recent studies have mainly examined short peptides^{4,5}, knowledge of the architecture of long polypeptide chains would be far more informative, but obtaining such information encounters two main roadblocks.

First, proteins adopt tertiary structures that prohibit nanopore translocation.

Second, unlike DNA or RNA, polypeptides have a low-density and heterogenous distribution of charge along their chains, which renders electrophoresis inapplicable as a means of translocation.

We have developed a general non-enzymatic means to map modifications within full-length polypeptide chains. Our methods can be used to inventory the collection of proteoforms in individual cells, rather than perform an ensemble analysis of peptide fragments (although this is not excluded).

Here, we use strong electroosmosis directly attributable to the charged side chains in an engineered αHL pore to capture long underivatized polypeptides and detect modifications within them as they are unfolded and translocated through protein nanopores.

### Methods

### Construction of Trx-Linker Concatamer Genes

All reagents were purchased from NEB (New England Biolabs) and DNA oligonucleotides were obtained from IDT (Integrated DNA Technologies) unless otherwise indicated. Trx-linker concatamer genes were prepared as previously described21. Briefly, the Trx-linker monomer gene was amplified with a 5′ primer containing a BamHI restriction site and a 3′ primer containing a BglII restriction site, which permitted in-frame cloning of the monomer into the vector pQE30 (Qiagen). The multi-domain synthetic gene was then constructed by iterative cloning of monomer into monomer, dimer into dimer, and tetramer into tetramer. To aid purification, an N-terminal SUMO tag was inserted between the His6 tag and the first monomer unit. In addition, N-terminal cysteine-glycine codons were included to give the final concatamer constructs: His6-SUMO-CysGly-(Trx-linker) n, n=2, 4, 6, and 8.

To produce Trx-linker nonamers (His6-SUMO-(Trx-linker) n, n=9) containing a modification site, the N-terminal cysteine-glycine codons were removed from the tetramer gene and a DNA cassette was designed to contain two terminal restriction sites (BamHI and BglII) and two internal restriction sites (KpnI and AvrII) (5′-pGATCCGGTGGTACCGGCGAGCTCGGTA-3′ (SEQ ID NO: 12), 5′-pGATCTACCGAGCTCGCCGGTACC ACCG-3′) (SEQ ID NO: 13). Using the interactive cloning strategy described above, a “cloneable” Trx-linker octamer gene was assembled with the DNA cassette as the middle unit flanked by two Trxlinker tetramer genes (i.e., the final construct is His6-SUMO-(Trx-linker)₄-KpnI-AvrII-(Trxlinker)₄). A Trx-linker monomer mutant gene containing the sequence of a RRASAC peptide motif (SEQ ID NO: 14) was created by site-directed insertion (Forward primer: 5′-AGCGCCTGCGCGGGTTCTGCTGGTTCC-3′, SEQ ID NO: 15; Reverse primer: 5′-CGCACGGCG GCTCCCTGCACTTCCGGC-3′, SEQ ID NO: 16) and subsequently cloned in between the KpnI and AvrII sites within the Trx-linker octamer to give (Trx-linker) 4-Trx-linker (RRASAC)-(Trx-linker) 4. The placement of a single correctly oriented insert was confirmed by sequencing using primers targeting the KpnI and AvrII ligation sites (Forward primer: 5′-TGCGAGCGCCTGCGGTGG3′, SEQ ID NO: 17; Reverse primer: 5′-ACGCTCGCGGACGCCACC-3′, SEQ ID NO: 18).

### Expression and Purification of Trx-Linker Concatamers

Genes encoding the N-terminal His6-SUMO tagged concatamers of Trx were cloned into the pOP3SU plasmid (kindly provided by Marko Hyvönen). BLR(DE3) competent cells (Novagen) were transformed with the plasmids and grown in Luria broth (LB) medium supplemented with ampicillin (100 μg/L) at 37° C. with continuous shaking (250 rpm). Protein expression was induced in the exponential growth phase (OD₆₀₀=0.6) with isopropyl-β-D-1-thiogalactopyranoside (IPTG) (0.5 mM final concentration). After 8 h, cells were harvested by centrifugation (10 min, 5,000 g), resuspended in binding buffer (30 mM Tris HCl, 250 mM NaCl, 25 mM imidazole, pH 7.2) supplemented with a protease inhibitor cocktail (cOmplete™, EDTA-free, Roche) and lysed by sonication. Cell debris was removed by centrifugation at 20,000 g for 45 min, and the supernatant loaded onto a His Trap HP column (5 mL, Cytiva) at 0.2 mL/min. The column was washed with 50 mL of the binding buffer before a single step elution with the elution buffer (30 mM Tris HCl, 250 mM NaCl, 300 mM imidazole, pH 7.2). A single peak containing the almost pure protein was collected and dialysed (Slide-A-Lyzer G2 Dialysis Cassette, 10,000 MWCO 30 mL, ThermoFisher) for 3 h against 4 L of dialysis buffer (50 mM Tris HCl, 250 mM NaCl, 2 mM 1,4-dithio-D-threitol (DTT), pH 8.0), at 4° C. with continuous stirring, to remove excess imidazole. After injecting the His6-tagged Ulp1 protease into the dialysis cassette at a molar concentration ratio of 1:200 (Ulp1: Trx-linker 12 concatamer), the mixture was transferred into fresh dialysis buffer overnight for SUMO-tag cleavage. The cassette was then transferred one last time into fresh dialysis buffer without DTT for 4 h. The dialysed protein was loaded onto a column packed with HisPur Ni-NTA Agarose Resin (5 mL, ThermoFisher) equilibrated with binding buffer (50 mM Tris HCl, 250 mM NaCl, pH 8.0) and the flow through was re-applied 5 more times. The final flow through containing the His6-SUMO-free protein was aliquoted and flash frozen for storage at −80° C.

### Expression and Purification of SUMO Protease Ulp1

The Pfget19_Ulp1 plasmid (Addgene) containing a His6-tagged Ulp1 gene was transformed into T7 Express competent cells (NEB) and grown in LB medium supplemented with ampicillin (100 μg/L) at 37° C. with shaking (250 rpm). Expression was induced at OD600=0.5 with IPTG (0.5 mM). Cells were harvested after 3 h by centrifugation, resuspended in lysis buffer (4 mL/g: 50 mM Tris HCl, 300 mM NaCl, 10 mM imidazole, pH 7.5) supplemented with lysozyme (1 mg/mL), and incubated on ice for 30 min before sonication. The lysate was spun at 20,000 rpm for 45 min to remove cell debris and the supernatant was applied to a column packed with HisPur Ni-NTA Agarose Resin (5 mL, ThermoFisher) and equilibrated with binding buffer (50 mM Tris HCl, 300 mM NaCl, pH 7.5). The column was washed with 10 column volumes of wash buffer (50 mM Tris HCl, 300 mM NaCl, 20 mM imidazole, pH 7.5) and the protein was eluted with 10 mL of elution buffer (50 mM Tris HCl, 300 mM NaCl, 300 mM imidazole, pH 7.5). The eluted protein was dialysed against storage buffer (50 mM Tris HCl, 200 mM NaCl, 2 mM 2-mercaptoethanol) overnight, aliquoted and flash frozen as a 50% stock in glycerol.

### Phosphorylation of Trx-Linker Concatamers

Trx-linker concatamers (1 mg/mL) were incubated with 50,000 units of the catalytic subunit of cAMP-dependent Protein Kinase (PKA) (NEB)—which recognizes the RRAS motif within the central linker of the Trx-linker nonamer—in protein kinase buffer (50 mM Tris HCl, pH 7.5.10 mM MgCl₂, 0.1 mM EDTA, 4 mM DTT, 0.01% Brij 35, and 2 mM ATP) (NEB) at 30° C. for 1 h. The solution was then supplemented with additional 2 mM ATP and 2 mM DTT before overnight incubation at 30° C. Trx-linker concatamers were purified and concentrated using centrifugal filters (Amicon Ultra-0.5 mL 100K), aliquoted and flash frozen for storage at −20° C. (10 mM HEPES, pH 7.2, and 750 mM KCl). Single phosphorylation of the Trx-linker concatamers was verified by LC-MS.

### Modification of Cysteines on Trx-Linker Concatamers

All reagents were purchased from Sigma-Aldrich unless otherwise indicated. Trx-linker nonamer was first treated with tris(2-carboxyethyl) phosphine (TCEP) (70 to 100 eq) at 32° C. for 2 h in protein storage buffer (50 mM Tris HCl, 250 mM NaCl, pH 8.0). Excess TCEP was removed by a desalting column (PD MiniTrap G-25 column, Cytiva). To glutathionylate Trxlinker nonamer, the reduced protein was reacted with oxidized glutathione (100 eq) at 32° C. overnight in protein storage buffer (50 mM Tris HCl, 250 mM NaCl, pH 8.0) before desalting to remove the excess reagent. The modified proteins were aliquoted and flash frozen for storage at −20° C. To glycosylate the Trx-linker nonamers, reduced protein was reacted first with 2,2′-dithiodipyridine (DPS) (20 eq) at 32° C. overnight in the protein storage buffer (50 mM Tris HCl, 250 mM NaCl, pH 8.0). After removal of excess DPS with a desalting column, the activated nonamer was reacted with the 6′-sialyllactosamine ligand (NeuAcα(2-6)LacNAc-PEG3-Thiol, 5 eq, Sussex Research Laboratories) overnight at 32° C. in protein storage buffer (50 mM Tris HCl, 250 mM NaCl, pH 8.0). Modified nonamers were desalted 13 (PD MiniTrap G-25 column, Cytiva), aliquoted and flash frozen for storage at −20° C. That glutathionylation or glycosylation occurred at single sites was verified by LC-MS mass spectrometry.

### Single-Channel Recording

Planar lipid bilayers of 1,2-diphytanoyl-sn-glycero-3-phosphocholine (Avanti Polar Lipids) were formed by using the Müller-Montal method on a 50 μm-diameter aperture made in a Teflon film (25 μm thick, Goodfellow) separating two 500 μL compartments (cis and trans) of the recording chamber. Each compartment was filled with recording buffer (750 mM GdnHCl, 1.5 M GdnHCl, 3 M GdnHCl, 2 M urea/750 mM KCl, or 750 mM KCl, 10 mM HEPES, 5 mM TCEP, pH 7.2 for Trx-linker dimer, tetramer, hexamer, and octamer; 375 mM GdnHCl/375 mM KCl, 10 mM HEPES, pH 7.2 for Trx-linker nonamers). To record with Trx-linker dimer, tetramer, hexamer, or octamer and ensure a reduced N-terminal cysteine, pre-treatment of the protein samples with 5 mM TCEP was carried out for 10 min at room temperature. Trx-linker concatamers were added to the cis compartment (dimer: 2.2 μM; tetramer: 0.63 μM; hexamer: 0.25 μM; octamer: 0.81 μM; nonamer: 1.2 μM). Ionic currents were measured at 24±1° C. by using Ag/AgCl electrodes connected to an Axopatch 200B amplifier. After a single (NN-113R)₇ pore had inserted into the bilayer, the solution was replaced with fresh buffer by manual pipetting, to prevent further insertions. Signals were low-pass filtered at 10 kHz and sampled at 50 kHz with a Digidata 1440A digitizer (Molecular Devices). Current traces were idealized by using Clampfit 10.3 (Molecular Devices). The idealized data were analyzed with QuB 2.0 software (www.qub.buffalo.edu). Dwell time analysis was performed by using the maximum interval likelihood algorithm of QuB.

### Results

We constructed dimers, tetramers, hexamers and octamers of thioredoxin (FIG. 2-3, Table 3). The thioredoxin (Trx, 108 amino acids) had the two catalytic cysteines removed (Trx: C32S/C35S)⁶. The Trx monomers were connected by 29-amino acid linkers, capable of spanning the 10-nm long lumen of the αHL nanopore when fully extended (0.35 nm per aa). We used an anion-selective αHL mutant, (NN_113R)₇ (P_{Na+}/P_{Cl-}=0.33), to generate electroosmosis¹³.

All four Trx-linker concatamers were captured by (NN_113R)₇ in the presence of 750 mM guanidinium chloride (GdnHCl) (FIG. 4) at a capture rate ˜25 times faster than that of a WT αHL pore (k(octamer) ˜2.5 s⁻¹ μM⁻¹ with (NN_113R)₇ versus ˜0.11 s⁻¹ μM⁻¹ with (WT)₇ at +140 mV).

TABLE 3
Sequences of the thioredoxin-linker concatamers

| | | SEQ ID NO |
|---|---|---|
| Dimer | CGSDKIIHLTDDSFDTDVLKADGAILVDFWAEWSGPSKMIAPILDEIADEYQGKLTVAKLNIDQNPGTAPKY | 3 |
| (Trx-linker)₂ | GIRGIPTLLLFKNGEVAATKVGALSKGQLKEFLDANLAGSAGSAGSAGSAGSAGSAGSAGSAGSAGRSDK | |
| | IIHLTDDSEDTDVLKADGAILVDFWAEWSGPSKMIAPILDEIADEYQGKLTVAKLNIDQNPGTAPKYGIRGIP | |
| | 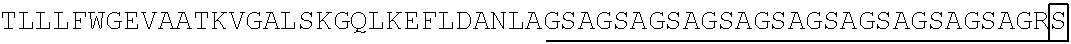 | |
| Tetramer | CGSDKIIHLTDDSFDTDVLKADGAILVDFWAEWSGPSKMIAPILDEIADEYQGKLTVAKLNIDQNPGTAPKY | 4 |
| (Trx-linker)₄ | GIRGIPTLLLFKNGEVAATKVGALSKGQLKEFLDANLAGSAGSAGSAGSAGSAGSAGSAGSAGSAGRSDK | |
| | IIHLTDDSFDTDVLKADGAILVDFWAEWSGPSKMIAPILDEIADEYQGKLTVAKLNIDQNPGTAPKYGIRGIP | |
| | TLLLFKNGEVAATKVGALSKGQLKEFLDANLAGSAGSAGSAGSAGSAGSAGSAGSAGSAGRSDKIIHLTD | |
| | DSFDTDVLKADGAILVDFWAEWSGPSKMIAPILDEIADEYQGKLTVAKLNIDQNPGTAPKYGIRGIPTLLLFK | |
| | NGEVAATKVGALSKGQLKEFLDANLAGSAGSAGSAGSAGSAGSAGSAGSAGSAGRSDKIIHLTDDSFDT | |
| | DVLKADGAILVDFWAEWSGPSKMIAPILDEIADEYQGKLTVAKLNIDQNPGTAPKYGIRGIPTLLLEKNGEVA | |
| | 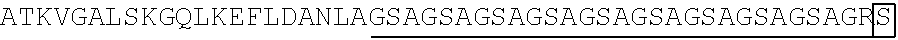 | |
| Hexamer | CGSDKIIHLTDDSFDTDVLKADGAILVDEWAEWSGPSKMIAPILDEIADEYQGKLTVAKLNIDQNPGTAPKY | 5 |
| (Trx-linker)₆ | GIRGIPTLLLFKNGEVAATKVGALSKGQLKEFLDANLAGSAGSAGSAGSAGSAGSAGSAGSAGSAGRSDK | |
| | IIHLTDDSFDTDVLKADGAILVDFWAEWSGPSKMIAPILDEIADEYQGKLTVAKLNIDQNPGTAPKYGIRGIP | |
| | TLLLFKNGEVAATKVGALSKGQLKEFLDANLAGSAGSAGSAGSAGSAGSAGSAGSAGSAGRSDKIIHLTD | |
| | DSFDTDVLKADGAILVDFWAEWSGPSKMIAPILDEIADEYQGKLTVAKLNIDQNPGTAPKYGIRGIPTLLLFK | |
| | NGEVAATKVGALSKGQLKEFLDANLAGSAGSAGSAGSAGSAGSAGSAGSAGSAGRSDKIIHLTDDSFDT | |
| | DVLKADGAILVDFWAEWSGPSKMIAPILDEIADEYQGKLTVAKLNIDONPGTAPKYGIRGIPTLLLFKNGEVA | |
| | ATKVGALSKGQLKEFLDANLAGSAGSAGSAGSAGSAGSAGSAGSAGSAGRSDKIIHLTDDSFDTDVLKAD | |
| | GAILVDEWAEWSGPSKMIAPILDEIADEYQGKLTVAKLNIDQNPGTAPKYGIRGIPTLLLFKNGEVAATKVG | |
| | ALSKGQLKEFLDANLAGSAGSAGSAGSAGSAGSAGSAGSAGSAGRSDKIIHLTDDSFDTDVLKADGAILV | |
| | DFWAEWSGPSKMIAPILDEIADEYQGKLTVAKLNIDQNPGTAPKYGIRGIPTLLLEKNGEVAATKVGALSKG | |
| | 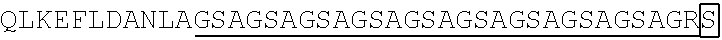 | |
| Octamer | CGSDKIIHLTDDSFDTDVLKADGAILVDFWAEWSGPSKMIAPILDEIADEYQGKLTVAKLNIDQNPGTAPKY | 6 |
| (Trx- | GIRGIPTLLLFKNGEVAATKVGALSKGQLKEFLDANLAGSAGSAGSAGSAGSAGSAGSAGSAGSAGRSDK | |
| linker)₇Trx | IIHLTDDSFDTDVLKADGAILVDFWAEWSGPSKMIAPILDEIADEYQGKLTVAKLNIDQNPGTAPKYGIRGIP | |
| | TLLLFKNGEVAATKVGALSKGQLKEFLDANLAGSAGSAGSAGSAGSAGSAGSAGSAGSAGRSDKIIHLTD | |
| | DSFDTDVLKADGAILVDFWAEWSGPSKMIAPILDEIADEYQGKLTVAKLNIDQNPGTAPKYGIRGIPTLLLFK | |
| | NGEVAATKVGALSKGQLKEFLDANLAGSAGSAGSAGSAGSAGSAGSAGSAGSAGRSDKIIHLTDDSEDT | |
| | DVLKADGAILVDFWAEWSGPSKMIAPILDEIADEYQGKLTVAKLNIDQNPGTAPKYGIRGIPTLLLFKNGEVA | |
| | ATKVGALSKGQLKEFLDANLAGSAGSAGSAGSAGSAGSAGSAGSAGSAGRSDKIIHLTDDSEDTDVLKAD | |
| | GAILVDFWAEWSGPSKMIAPILDEIADEYQGKLTVAKLNIDQNPGTAPKYGIRGIPTLLLFKNGEVAATKVG | |
| | ALSKGQLKEFLDANLAGSAGSAGSAGSAGSAGSAGSAGSAGSAGRSDKIIHLTDDSFDTDVLKADGAILV | |
| | DFWAEWSGPSKMIAPILDEIADEYQGKLTVAKLNIDQNPGTAPKYGIRGIPTLLLEKNGEVAATKVGALSKG | |
| | QLKEFLDANLAGSAGSAGSAGSAGSAGSAGSAGSAGSAGRSDKIIHLTDDSEDTDVLKADGAILVDEWAE | |
| | WSGPSKMIAPILDEIADEYQGKLTVAKLNIDQNPGTAPKYGIRGIPTLLLEKNGEVAATKVGALSKGQLKEFL | |
| | DANLAGSAGSAGSAGSAGSAGSAGSAGSAGSAGRSDKIIHLTDDSEDTDVLKADGAILVDFWAEWSGPS | |
| | KMIAPILDEIADEYQGKLTVAKLNIDQNPGTAPKYGIRGIPTLLLEKNGEVAATKVGALSKGQLKEFLDANLA | |
| (Trx-linker)₄ | SDKIIHLTDDSFDTDVLKADGAILVDFWAEWSGPSKMIAPILDEIADEYQGKLTVAKLNIDQNPGTAPKYGIR | 7 |
| (Trx-linker- | GIPTLLLFKNGEVAATKVGALSKGQLKEFLDANLAGSAGSAGSAGSAGSAGSAGSAGSAGSAGRSDKIIHL | |
| 24S/26C) | TDDSFDTDVLKADGAILVDFWAEWSGPSKMIAPILDEIADEYQGKLTVAKLNIDQNPGTAPKYGIRGIPTLLL | |
| (Trx-linker)₄ | FKNGEVAATKVGALSKGQLKEFLDANLAGSAGSAGSAGSAGSAGSAGSAGSAGSAGRSDKIIHLTDDSF | |
| | DTDVLKADGAILVDEWAEWSGPSKMIAPILDEIADEYQGKLTVAKLNIDQNPGTAPKYGIRGIPTLLLFKNGE | |
| | VAATKVGALSKGQLKEFLDANLAGSAGSAGSAGSAGSAGSAGSAGSAGSAGRSDKIIHLTDDSFDTDVLK | |
| | ADGAILVDFWAEWSGPSKMIAPILDEIADEYQGKLTVAKLNIDQNPGTAPKYGIRGIPTLLLFKNGEVAATKV | |
| | 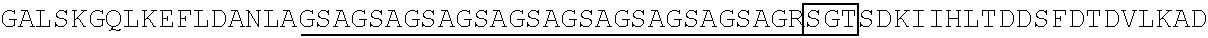 | |
| | GAILVDFWAEWSGPSKMIAPILDEIADEYQGKLTVAKLNIDQNPGTAPKYGIRGIPTLLLFKNGEVAATKVG | |
| | 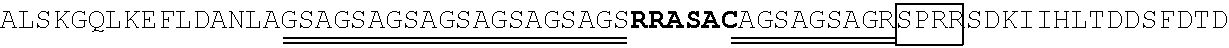 | |
| | VLKADGAILVDFWAEWSGPSKMIAPILDEIADEYQGKLTVAKLNIDQNPGTAPKYGIRGIPTLLLFKNGEVAA | |
| | TKVGALSKGQLKEFLDANLAGSAGSAGSAGSAGSAGSAGSAGSAGSAGRSDKIIHLTDDSFDTDVLKADG | |
| | AILVDFWAEWSGPSKMIAPILDEIADEYQGKLTVAKLNIDQNPGTAPKYGIRGIPTLLLFKNGEVAATKVGAL | |
| | SKGQLKEFLDANLAGSAGSAGSAGSAGSAGSAGSAGSAGSAGRSDKIIHLTDDSFDTDVLKADGAILVDF | |
| | WAEWSGPSKMIAPILDEIADEYQGKLTVAKLNIDQNPGTAPKYGIRGIPTLLLEKNGEVAATKVGALSKGQL | |
| | KEFLDANLAGSAGSAGSAGSAGSAGSAGSAGSAGSAGRSDKIIHLTDDSFDTDVLKADGAILVDFWAEWS | |
| | GPSKMIAPILDEIADEYQGKLTVAKLNIDQNPGTAPKYGIRGIPTLLLFKNGEVAATKVGALSKGQLKEFLDA | |
| | 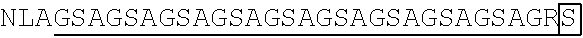 | |
| (Trx-linker), | SDKIIHLTDDSFDTDVLKADGAILVDFWAEWSGPSKMIAPILDEIADEYQGKLTVAKLNIDNPGTAPKYGIR | 8 |
| (Trx-linker- | GIPTLLLFKNGEVAATKVGALSKGQLKEFLDANLAGSAGSAGSAGSAGSAGSAGSAGSAGSAGRSDKIIHL | |
| 14S/16C) | TDDSFDTDVLKADGAILVDFWAEWSGPSKMIAPILDEIADEYQGKLTVAKNNIDQNPGTAPKYGIRGIPTLLL | |
| (Trx-linker)4 | FKNGEVAATKVGALSKGQLKEFLDANLAGSAGSAGSAGSAGSAGSAGSAGSAGSAGRSDKIIHLTDDSF | |
| | DTDVLKADGAILVDFWAEWSGPSKMIAPILDEIADEYQGKLTVAKLNIDQNPGTAPKYGIRGIPTLLLFKNGE | |
| | VAATKVGALSKGQLKEFLDANLAGSAGSAGSAGSAGSAGSAGSAGSAGSAGRSDKIIHLTDDSFDTDVLK | |
| | ADGAILVDFWAEWSGPSKMIAPILDEIADEYQGKLTVAKLNIDQNPGTAPKYGIRGIPTLLLFKNGEVAATKV | |
| | 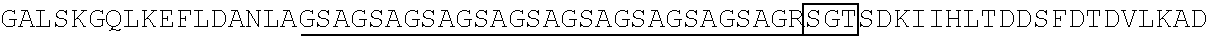 | |
| | GAILVDFWAEWSGPSKMIAPILDEIADEYQGKLTVAKLNIDQNPGTAPKYGIRGIPTLLLFKNGEVAATKVG | |
| | 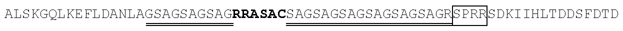 | |
| | VLKADGAILVDFWAEWSGPSKMIAPILDEIADEYQGKLTVAKLNIDQNPGTAPKYGIRGIPTLLLFKNGEVAA | |
| | TKVGALSKGQLKEFLDANLAGSAGSAGSAGSAGSAGSAGSAGSAGSAGRSDKIIHLTDDSFDTDVLKADG | |
| | AILVDFWAEWSGPSKMIAPILDEIADEYQGKLTVAKLNIDQNPGTAPKYGIRGIPTLLLFKNGEVAATKVGAL | |
| | SKGQLKEFLDANLAGSAGSAGSAGSAGSAGSAGSAGSAGSAGRSDKIIHLTDDSFDTDVLKADGAILVDF | |
| | WAEWSGPSKMIAPILDEIADEYQGKLTVAKLNIDQNPGTAPKYGIRGIPTLLLFKNGEVAATKVGALSKGQL | |
| | KEFLDANLAGSAGSAGSAGSAGSAGSAGSAGSAGSAGRSDKIIHLTDDSFDTDVLKADGAILVDFWAEWS | |
| | GPSKMIAPILDEIADEYQGKLTVAKLNIDQNPGTAPKYGIRGIPTLLLFKNGEVAATKVGALSKGQLKEFLDA | |
| | 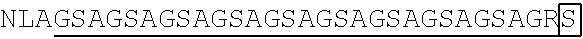 | |

Underlined = linker
Double underlined = modified linker
Italic = N-terminal cysteine-glycine 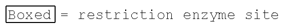
Bold = sequence for modification

Electroosmosis-driven concatamer translocation produced current patterns containing repeating features (FIG. 4, FIGS. 8-9). The most abundant feature, A, consisted of three levels (A1, A2, A3) (FIG. 4-5). The percentage residual current (I_{res %}) for each level in feature A was consistent across all such events for each polypeptide translocation and between all individual concatamers observed with the same or different pores (Table 4). A spike to ˜0 pA was seen at the beginning of almost all the translocation events and was speculated to represent the rapid unfolding and translocation of the first Trx-linker unit. The spike was followed by up to n−1 repeats of the three-step feature A (n=number of Trx-linker units in the concatamer), which unambiguously demonstrated the stepwise translocation of entire polypeptide chains one unit at a time.

TABLE 4
Percentage residual currents (Ires %) for the three
levels of repeating feature A recorded during
C-terminus first concatamer translocation.

| | Trx-linker | Trx-linker | Trx-linker |
|---|---|---|---|
| | dimer | tetramer | octamer |
| I_{res %} (A1)^{[a]} | 34 ± 1% | 35 ± 1% | 35 ± 1% |
| I_{res %} (A2)^{[a]} | 22 ± 3% | 24 ± 2% | 23 ± 1% |
| I_{res %} (A3)^{[a], [b]} | 1.9 ± 2.4% | 1.7 ± 1.7% | 2.3 ± 2.7% |
| N^{[a], [b]} | 105 units | 66 units | 443 units |
| | 2 separate pores | 3 separate pores | 2 separate pores |

^{[a]}Ires % was calculated for each step in individual features A as the remaining current as a percentage of the open pore current (e.g., I_{res %}(A1) = I_{A1}/Iₒₚₑₙ). The standard deviations were derived for N Trx-linker units collected with >1 separate pores. Conditions: 750 mM GdnHCl, 10 mM HEPES, pH 7.2, +140 mV (trans), 24 ± 1° C.
^{[b]}Trx-linker units that produced a Level A3 with a dwell time 1 ms were discarded during analysis. The associated spikey appearance suggested under-sampling and therefore an inaccurate I_{res %} value. Level A3 with a dwell time >1 ms and a square shape

Less often, a different repeating element, B, was recorded (FIG. 8). Further, when two identical concatamers were linked by a disulfide bond between the N-terminal cysteines, feature B occurred only after feature A within each translocation event (FIG. 9). Therefore, we assigned these two features as C terminus-first (A) and N terminus-first (B) translocation events. Previously, electrophoresis-driven translocation of Trx monomers tagged with a DNA leader at either the N or C terminus was reported to proceed through two steps in consistence with features A and B8.

The repeating feature A was lost at a GdnHCl concentration of 3 M (FIG. 10). At 750 mM GdnHCl, ˜12% of the translocated octamers produced the maximum of 7 repeats of feature A following the initial spike; kinetic analysis revealed two populations of A3: one had a mean dwell time ˜500 times longer than the other at +140 mV (<τ_{A3}>=320±60 ms versus 0.69±0.04 ms) (Table 5). The longer-lived A3 (τ_{A3}>10 ms) was seen in 25% of the final features A recorded before translocation of an octamer was complete, but only in 3% of the preceding features A. Tentatively, we assign Level A1 as a threaded linker preceding the C-terminus of a folded Trx unit; Level A2 as a C-terminal portion of a partially unfolded Trx unit extended into the nanopore; Level A3 as the spontaneous unfolding and passage of the remaining Trx polypeptide through the nanopore (FIG. 5). The absence of a multi-level feature for the first unit and an extended duration for the last unit suggest that the unfolding kinetics of Trx units differ when the polypeptide chain is unable to fully span the lumen of the nanopore.

TABLE 5
Mean dwell times (<τ>) derived by QuB[^{a}] for the
three levels of repeating feature A (A1, A2, A3)
recorded during the C-terminus first translocation of
Trx-linker octamers through a single (NN_113R)₇
nanopore[^{b}].

| | | |
|---|---|---|
| <τ_{A1}>/ms | 270 ± 20 | n = 277 |
| <τ_{A2}>/ms | 23 ± 1 | n = 277 |
| <τ_{A3}>/ms | 320 ± 60 | n = 40 |
| | 0.69 ± 0.04 | n = 294 |

[^{a}]Dwell time analysis was performed by using the maximum interval likelihood algorithm of QuB.
[^{b}]Conditions: 750 mM GdnHCl, 10 mM HEPES, pH 7.2, +140 mV (trans), 24 ± 1° C.

To determine whether PTMs near the middle of a long polypeptide chain could be located during electroosmosis-driven translocation, we constructed Trx-linker nonamers containing a modification site (RRASAC) at two different positions in the central linker (Table 3) for serine phosphorylation (14S-P or 24S-P) or cysteine-directed glutathionylation or glycosylation (16C-GSH, 26C-GSH, 16C-SLN, or 26C-SLN) (FIG. 6). In the presence of a phosphate group (P) or glutathione (GSH) or 6′-sialyllactosamine (SLN), Level A1 for the modified units exhibited a smaller I_{res %} and higher root-mean-square noise (I_{RMS}) than that of unmodified segments within an individual polypeptide (FIG. 7, Table 6). The average increment in the current blockade was roughly proportional to the mass of the PTM with phosphate giving the smallest increment and the trisaccharide the largest (Table 6), although there was substantial overlap between the 14S-P/24SP and 16C-GSH/26C-GSH populations (FIG. 7, FIG. 11). All three PTMs tested caused smaller current blockade at serine 14 (14S) or cysteine 16 (16C) than at serine 24 (24S) or cysteine 26 (26C) (FIG. 7). Given that 14S/16C must be closer to the cis opening of the αHL pore than 24S/26C in a C terminus first threading configuration, it is likely that the central constriction of the pore is located closer to 24S/26C (FIG. 6, FIG. 12). The findings also suggest that the polypeptide might not be fully extended under the EOF (See FIG. 12 for further analysis), which corroborates force spectroscopy data for polypeptides under low pN forces¹⁸.

TABLE 6
Percentage residual current (Ires %) and root-
mean-square noise (I_{RMS}) characteristics of individual
modifications on Trx-linker nonamers.

| | ΔI_{res %}^{[a]} | I_{RMS}/pA^{[c]} | N |
|---|---|---|---|
| Trx-linker-14S-P | 4.4 ± 0.8% | 0.96 ± 0.18 | 19 concatemers |
| | | | 4 separate pores |
| | 4.0 ± 1.2%^{[b]} | 1.6 ± 0.9^{[b]} | 19 concatemers |
| | | | 4 separate pores |
| Trx-linker-24S-P | 8.3 ± 1.6% | 2.0 ± 0.6 | 27 concatemers |
| | | | 3 separate pores |
| | 9.2 ± 2.1%^{[b]} | 2.5 ± 0.9^{[b]} | 23 concatemers |
| | | | 3 separate pores |
| Trx-linker-16C-GSH | 5.1 ± 0.9% | 0.93 ± 0.19 | 46 concatemers |
| | | | 4 separate pores |
| Trx-linker-26C-GSH | 8.6 ± 1.3% | 1.6 ± 0.2 | 23 concatemers |
| | | | 3 separate pores |
| Trx-linker-16C-SLN | 15 ± 1% | 0.73 ± 0.28 | 24 concatemers |
| | | | 3 separate pores |
| Trx-linker-26C-SLN | 18 ± 2% | 1.8 ± 0.6 | 55 concatemers |
| | | | 5 separate pores |

^{[a]}ΔIres % = <I_{res %}(A1, Trx-linker) − I_{res %}(A1, Trx-linker + PTM). For a C terminus-first translocation event, <I_{res %}(A1, Trx-linker)> was determined as the mean Ires % value of the remaining A1 levels within an individual translocation event. Ires %(A1, Trx-linker + PTM) was determined for the A1 level of the modified linker and appeared once per translocating concatamer. Conditions: 375 mM GdnHCl, 375 mM KCl, 10 mM HEPES, pH 7.2, +140 mV (trans), 24 ± 1° C.
^{[b]}Conditions: 750 mM GdnHCl, 10 mM HEPES, pH 7.2, +140 mV (trans), 24 ± 1° C.
^{[c]}Root-mean-square noise values (I_{RMS}) were measured from current traces after an applied post-recording filter at 2 kHz. I_{RMS} was normalised by the noise of each pore (I_{RMS}² = I_{RMS}(A1, Trx-linker + PTM)² − I_{RMS}(open pore)²).

### CONCLUSIONS

Here, we have established that electroosmotically active nanopores can capture and unfold individual proteins comprising long (>1200 aa) polypeptide chains for PTM identification and localisation. To a first approximation, the electroosmotic force acting on a polypeptide remains constant during translocation, which creates a unidirectional bias desirable for the placement of PTMs in sequence. In contrast, the overall time for unforced polypeptide translocation scales roughly as the square of its length, because the polypeptide chain can move back and forth before diffusing out of the pore¹⁹. This is the case within a electroosmotically-inactive nanopore after the exit of a charged leader sequence⁶ or immediately after a protein domain has unfolded during movement propelled by a motor protein⁹, which is not ideal for the sequential detection of modification sites within individual polypeptide chains. As a label-free method, our approach circumvents the need to derivatize proteins at either the N or C terminus for electrophoretic translocation, which could be problematic for eukaryotic proteins due to the widespread presence of N-acetylation and the lack of efficient N or C terminus-specific chemistries. Although we have located PTMs in linkers within a polyprotein chain, PTMs in folded proteins can be detected in an analogous way during electroosmotic co-translocational unfolding of protein domains. Our strategy will be readily transferable to nanopore sequencing devices (e.g., the MinION) for highly parallel PTM profiling, which will be useful for producing inventories of full-length human proteoforms, which are ˜500 aa in median length²⁰. To promote characterisation of the proteoforms in individual cells, voltage sweepsmay be used in combination with denaturants to promote protein capture and enable cotranslocational unfolding. Ligand-assisted detection may be assisted by the use of antibodies or chemical binders.

In summary, our enzyme-less approach, targeting full-length proteins, presents a useful nanopore technology, which will ultimately allow comprehensive proteoform inventories to be established for tissues and single cells. These massive sets of information will extend beyond what is recognised from DNA and RNA sequencing, and will potentially unveil as yet unknown aspects of the biology of cells and tissues

### REFERENCES FOR EXAMPLE 1

1. Wang, Y., Zhao, Y., Bollas, A., Wang, Y. & Au, K. F. Nanopore sequencing technology, bioinformatics and applications. Nat. Biotechnol. 39, 1348-1365 (2021).
2. Restrepo-Pérez, L., Joo, C. & Dekker, C. Paving the way to single-molecule protein sequencing. Nat. Nanotechnol. 13, 786-796 (2018).
3. Sharma, K. et al. Ultradeep human phosphoproteome reveals a distinct regulatory nature of Tyr and Ser/Thr-based signaling. Cell Rep. 8, 1583-1594 (2014).
4. Brinkerhoff, H., Kang, A. S. W., Liu, J., Aksimentiev, A. & Dekker, C. Multiple rereads of single proteins at single-amino acid resolution using nanopores. Science 374, 1509-1513 (2021).
5. Lucas, F. L. R., Versloot, R. C. A., Yakovlieva, L., Walvoort, M. T. C. & Maglia, G. Protein identification by nanopore peptide profiling. Nat. Commun. 12, 1-9 (2021).
6. Rodriguez-Larrea, D. & Bayley, H. Multistep protein unfolding during nanopore translocation. Nat. Nanotechnol. 8, 288-95 (2013).
7. Rosen, C. B., Rodriguez-Larrea, D. & Bayley, H. Single-molecule site-specific detection of protein phosphorylation with a nanopore. Nat. Biotechnol. 32, 179-181 (2014).
8. Rodriguez-Larrea, D. & Bayley, H. Protein co-translocational unfolding depends on the direction of pulling. Nat. Commun. 5, 4841 (2014).
9. Nivala, J., Marks, D. B. & Akeson, M. Unfoldase-mediated protein translocation through an α-hemolysin nanopore. Nat. Biotechnol. 31, 247-250 (2013).
10. Zhang, S. et al. Bottom-up fabrication of a proteasome-nanopore that unravels and processes single proteins. Nat. Chem. 13, 1192-1199 (2021).
11. Cockroft, S. L., Chu, J., Amorin, M. & Ghadiri, M. R. A single-molecule nanopore device detects DNA polymerase activity with single-nucleotide resolution. J. Am. Chem. Soc. 130, 818-820 (2008).
12. Cherf, G. M. et al. Automated forward and reverse ratcheting of DNA in a nanopore at 5-Å precision. Nat. Biotechnol. 30, 344-348 (2012).
13. Gu, L.-Q. L. Q., Cheley, S. & Bayley, H. Electroosmotic enhancement of the binding of a neutral molecule to a transmembrane pore. Proc. Natl. Acad. Sci. U.S.A. 100, 15498-15503 (2003).
14. Huang, G. et al. Electro-osmotic vortices promote the capture of folded proteins by plyAB nanopores. Nano Lett. 20, 3819-3827 (2020).
15. Huang, G., Willems, K., Soskine, M., Wloka, C. & Maglia, G. Electro-osmotic capture and ionic discrimination of peptide and protein biomarkers with FraC nanopores. Nat. Commun. 8, 935 (2017).
16. Asandei, A. et al. Electroosmotic trap against the electrophoretic force near a protein nanopore reveals peptide dynamics during capture and translocation. ACS Appl. Mater. Interfaces 8, 13166-13179 (2016).
17. Yu, L. et al. Unidirectional single-file transport of full-length proteins through a nanopore. bioRxiv 2021.09.28.462155 (2021). doi: 10.1101/2021.09.28.462155
18. Winardhi, R. S., Tang, Q., Chen, J., Yao, M. & Yan, J. Probing small molecule binding to unfolded polyprotein based on its elasticity and refolding. Biophys. J. 111, 2349-2357 (2016).
19. Palyulin, V. V., Ala-Nissila, T. & Metzler, R. Polymer translocation: The first two decades and the recent diversification. Soft Matter 10, 9016-9037 (2014).
20. Brocchieri, L. & Karlin, S. Protein length in eukaryotic and prokaryotic proteomes. Nucleic Acids Res. 33, 3390-3400 (2005).
21. Carrion-Vazquez, M. et al. Mechanical and chemical unfolding of a single protein: A comparison. Proc. Natl. Acad. Sci. U.S.A. 96, 3694-3699 (1999).
22. Winardhi, R. S., Tang, Q., Chen, J., Yao, M. & Yan, J. Probing small molecule binding to unfolded polyprotein based on its elasticity and refolding. Biophys. J. 111, 2349-2357 (2016).
23. Howorka, S. & Bayley, H. Probing distance and electrical potential within a protein pore with tethered DNA. Biophys. J. 83, 3202-3210 (2002).

### Example 2

The detection and mapping of protein post-translational modification sites such as phosphorylation sites are essential for understanding the mechanisms of various cellular processes and for identifying targets for drug development. The study of biopolymers at the single-molecule level has been revolutionized by nanopore technology.

In this study, we detect protein phosphorylation (as an exemplary PTM) within long polypeptides, after the attachment of phosphate monoester-specific chemical binders, by using electro-osmosis to drive the tagged chains through engineered protein nanopores. By monitoring the ionic current carried by a nanopore, phosphorylation sites are located within individual polypeptide chains, providing a valuable step toward nanopore proteomics.

### Introduction

Post-translational modifications (PTMs) of proteins are pivotal in cell regulation and typically involve the enzymatic addition of chemical groups to amino acid side chains¹. Phosphorylation, a dominant PTM, is associated with diseases such as cancer, Parkinson's, and Alzheimer's². Bottom-up mass spectrometry is routinely applied to detect PTMs on peptide fragments derived from disease-related proteins, but faces challenges to determine if widely separated modifications, whether identical or distinct, are present on the same polypeptide chain. For example, the cross-talk between phosphorylation and O-GlcNAcylation was reported to regulate subcellular localization of proteins, such as tau³. However, there lacks a straightforward technique to correlate their presence at distant sites at the single-protein level⁴. Nanopore nucleic acid sequencing has emerged as a powerful technology to provide ultra-long DNA or RNA reads for long-range correlation of genomic or transcriptomic features^{5,6}. Single-molecule sensing using protein nanopores therefore holds great potential for single-molecule analysis of full-length proteoforms⁷⁻¹¹ Efforts have been made to propel unfolded polypeptides through nanopores¹²⁻¹⁴ and PTMs deep within long polypeptide chains have been located during translocation¹³. This work is a first step towards the label-free analysis of modified proteins extracted from biological samples¹³. In parallel, efforts to identify PTMs on short peptides (up to ˜30 amino acids) have been described¹⁵⁻¹⁷, either when the peptides are sensed as a whole or when a peptide is transported through the pore as a conjugate to an oligonucleotide¹⁷.

Previously, we detected three PTMs (phosphorylation, glutathionylation, and glycosylation) on full-length proteins when segments of singly modified thioredoxin (Trx)-linker concatemers were stalled during translocation through a nanopore¹³. To our surprise, the glutathionylation and phosphorylation, placed at sites two amino acids apart, produced similar current blockades and noise patterns¹³.

To facilitate distinguishing PTMs of similar electrical signatures or to allow targeted detection of specific PTMs, we here seek to use PTM-specific binders to generate distinct current characteristics. To this end, we have explored a phosphorylation-specific reversible chemical binder, Phos-tag, which binds selectively and strongly to phosphate monoesters when complexed with zinc ions (e.g., for phosphoserine or phosphotheronine residues within model peptides, K_{d}=˜0.7 μM; for phosphotyrosine residues within model peptides, K_{d}=˜70 nM; for SO₄²⁻, K_{d}=˜130 μM; for Cl⁻, K_{d}=˜2 mM)^{18,19}. Phos-tag produced distinctive modulation of the associated ionic current as phosphorylated polypeptide chains were translocated through an engineered nanopore, allowing the location of phosphorylation sites within long polypeptide chains. Whilst this example describes the use of phos-tag as an exemplary binder for phosphorylation, the concepts discussed herein are widely applicable to detection of a wide range of post-translational modifications using appropriate binders known in the art.

### Results and Discussion

In our previous research, we employed an anion-selective α-hemolysin (αHL) mutant (NN-113R)₇ (permeability ratio P_{Na+}/P_{Cl-}=0.33)²⁰ to generate electro-osmotic flow, thereby driving the capture, linearization, and translocation of polypeptide chains. We identified and located PTMs on long polypeptide chains of up to nine thioredoxin units (Trx, 108 amino acids (aa)) connected by linkers (29 aa)¹³. Each Trx units within the Trx-linker concatemers had the two catalytic cysteines removed (Trx: C32S/C35S)⁷. Chaotropic reagents (e.g. guanidinium chloride, GdnHCl, or urea) at non-denaturing concentrations were used to promote the co-translocational unfolding¹³. During the electro-osmotic translocation of the Trx-linker concatemers, features comprising three levels were seen (A1, A2 and A3) (FIG. 1a, b). We provisionally assigned level A1 to be produced by the nanopore containing a threaded linker ahead of a folded Trx unit, level A2 to be produced when a partly unfolded C-terminus of a Trx unit extended into the nanopore, and level A3 to be produced by the spontaneous unfolding and passage of the remaining Trx polypeptide chain through the nanopore. In the presence of a PTM in the linker, a phosphate group (P) for instance, level A1 exhibited a smaller percentage residual current (I_{res %}) value and higher root-mean-square noise (I_{r.m.s.})¹³ (FIG. 1b).

Here, we have examined the detection of phosphorylation in association with a phosphate-specific binder: Phos-tag-acrylamide dizinc complex (PAZn₂). We constructed a Trx-linker pentamer containing two phosphorylation sites (RRAS) in the second and fourth linkers (FIG. 1a, Table 7 and FIG. 15), which were phosphorylated on serine by the catalytic subunit of protein kinase A (FIG. 16). Phosphorylated polypeptides were captured, unfolded, and translocated by electro-osmosis through the (NN-113R)₇ αHL pore. GdnHCl (750 mM) was employed to accelerate the co-translational unfolding. Consistent with prior findings, translocation of the pentamer, C-terminus first, generated current patterns with a maximum of 4 A1-A3 repeats following an initial spike (FIG. 1b). The spike to around 0 pA at the beginning of nearly all the translocation events was attributed to rapid unfolding and translocation of the first C-terminal Trx-linker unit. While only ˜6% of the doubly phosphorylated Trx-linker pentamers produced 5 repeating A1-A3 features, >72% of the recorded translocation events contained at least one A1 level with a reduced I_{res %} value and a higher I_{r.m.s.}, compared to A1 levels for unmodified segments (Table S2). These characteristics aligned with the electrical profiles previously identified for a phosphorylated linker and therefore assigned as level A1-P. In events where 5 repeats of A1-A3 features were observed, the level A1-P was recorded for both the second and fourth units, consistent with the presence of two phosphorylated serine residues (Ser-P) within the second and fourth linkers, 274 amino acids apart within the polypeptide chain.

To determine whether the binding of PAZn₂ to phosphates in the polypeptide chains could be identified during translocation, we pre-formed complexes of phosphorylated Trx-linker pentamer with PAZn₂ with a molar ratio of Trx-linker:Phos-tag-acrylamide:ZnCl₂=1:50:100, and added the mixture to the cis compartment of the recording chamber. While the unmodified segments exhibited A1 levels characteristic of the unphosphorylated linkers, ˜80% of the phosphorylated linkers generated a distinctive A1 state with an ionic current that alternated between two levels (FIG. 1c, 17). To verify whether the distinctive current feature stemmed from the association of PAZn₂ and Ser-P in the Trx-linker pentamer, a competition assay was performed in which excess phosphoserine was introduced to compete for binding with PAZn₂ (Methods and FIG. 19). Nanopore characterization of the phosphorylated Trx-linker pentamers in complex with PAZn₂ (preformed at a molar ratio of Trx-linker:Phos-tag-acrylamide:ZnCl₂=1:50:100) was first recorded for approximately 10 minutes (Methods). Subsequently, excess phosphoserine (100 eq.) was added to the cis compartment, and another 10-minute recording was performed. Prior to the addition of phosphoserine, 79% of the A1-P levels (N=29) exhibited two alternating steps. The frequency of these events dropped to 16% (N=24) after the addition of phosphoserine, suggesting that state A1 with two interconverting levels arose from the binding of PAZn₂ to Ser-P (henceforth A1-P-PAZn₂). Transitions between a A1-P-PAZn₂ level and a level with an ionic current closely similar to level A1-P were also detected (FIG. 20), which was attributed to the dissociation of PAZn₂ from Ser-P while the phosphorylated polypeptide segment was within the pore. The two current levels in A1-P-PAZn₂ likely reflect the two-step chelation of a phosphate monoester with PAZn₂²¹⁻²³. A kinetics analysis revealed that the level with larger current blockades (A1-P-PAZn₂-L) had a mean dwell time that was ˜4 times longer than the level with smaller current blockades (A1-P-PAZn₂-H) (=11.6±0.3 ms, =3.3±0.1 ms), indicating that level A1-P-PAZn₂-L was the more stable binding state (Table S3). We suggest that level A1-P-PAZn₂-L represents PAZn₂ with both zinc ions chelated by phosphate oxygen atoms, and level A1-P-PAZn₂-H, PAZn₂ with only one zinc ion chelated by a phosphate oxygen atom.

Next, we sought to determine if PAZn₂ would enable us to distinguish phosphorylation from a PTM that exhibits a similar ionic blockade¹³. We constructed a Trx-linker pentamer with distinct modification sites in the second (RRASAA) and fourth (RRAAAC) linkers. We carried out phosphorylation and glutathionylation reactions sequentially to obtain a Trx-linker pentamer with Ser-P in the second linker and glutathionylated cysteine (Cys-GS) in the fourth linker (FIG. 2a). In line with the characteristic current patterns recorded separately with Trx-linker nonamers containing a single Ser-P or Cys-GS residues within the same linker sequence¹³, the signals from Ser-P and Cys-GS within the same Trx-linker pentamer exhibited indistinguishable residual currents and noise when the second and fourth linkers were located within the pore (FIG. 2a). Pleasingly, the introduction of PAZn₂ altered the signal derived from the second linker to give a pattern similar to level A1-P-PAZn₂, while the signal from the fourth linker was unchanged, allowing clear differentiation between phosphorylation and glutathionylation (FIG. 2b).

TABLE 7
Sequences of the thioredoxin-linker concatamers

| | | SEQ ID NO |
|---|---|---|
| (Trx- | SDKIIHLTDDSEDTDVLKADGAILVDEWAEWSGPSKMIAPILDEIADEYQGKLTVAKLNIDQNPGT | 19 |
| linker)_{1,3,5} | APKYGIRGIPTLLLEKNGEVAATKVGALSKGQLKEFLDANLAGSAGSAGSAGSAGSAGSAGSAG | |
| (Trx-linker- | SAGSAGRSDKIIHLTDDSEDTDVLKADGAILVDFWAEWSGPSKMIAPILDEIADEYQGKLTVAKLN | |
| 24S26C)_{2,4} | IDQNPGTAPKYGIRGIPTLLLEKNGEVAATKVGALSKGQLKEFLDANLAGSAGSAGSAGSAGSA | |
| | GSAGSRRASACAGSAGSAGRSDKIIHLTDDSEDTDVLKADGAILVDFWAEWSGPSKMIAPILDEI | |
| | ADEYQGKLTVAKLNIDQNPGTAPKYGIRGIPTLLLEKNGEVAATKVGALSKGOLKEFLDANLAGS | |
| | 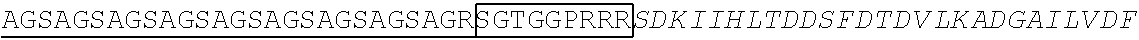 | |
| | WAEWSGPSKMIAPILDEIADEYQGKLTVAKLNIDQNPGTAPKYGIRGIPTLLLFKNGEVAATKVG | |
| | ALSKGQLKEFLDANLAGSAGSAGSAGSAGSAGSAGSRRASACAGSAGSAGRSDKIIHLTDDSF | |
| | DTDVLKADGAILVDEWAEWSGPSKMIAPILDEIADEYQGKLTVAKLNIDQNPGTAPKYGIRGIPTL | |
| | LLFKNGEVAATKVGALSKGOLKEFLDANLAGSAGSAGSAGSAGSAGSAGSAGSAGSAGRS | |
| (Trx- | SDKIIHLTDDSFDTDVLKADGAILVDEWAEWSGPSKMIAPILDEIADEYQGKLTVAKLNIDONPGT | 20 |
| linker)_{1,3,5} | APKYGIRGIPTLLLEKNGEVAATKVGALSKGQLKEFLDANLAGSAGSAGSAGSAGSAGSAGSAG | |
| (Trx-linker- | SAGSAGRSDKIIHLTDDSFDTDVLKADGAILVDFWAEWSGPSKMIAPILDEIADEYQGKLTVAKLN | |
| 24S)₂(Trx- | IDQNPGTAPKYGIRGIPTLLLFKNGEVAATKVGALSKGQLKEFLDANLAGSAGSAGSAGSAGSA | |
| linker-26C)₄ | GSAGSRRASAAAGSAGSAGRSDKIIHLTDDSEDTDVLKADGAILVDFWAEWSGPSKMIAPILDEI | |
| | ADEYQGKLTVAKLNIDQNPGTAPKYGIRGIPTLLLEKNGEVAATKVGALSKGQLKEFLDANLAGS | |
| | 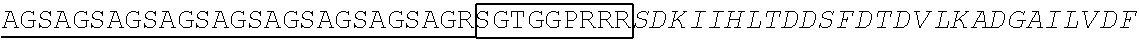 | |
| | WAEWSGPSKMIAPILDEIADEYQGKLTVAKLNIDQNPGTAPKYGIRGIPTLLLEKNGEVAATKVG | |
| | ALSKGQLKEFLDANLAGSAGSAGSAGSAGSAGSAGSRRAAACAGSAGSAGRSDKIIHLTDDSF | |
| | DTDVLKADGAILVDEWAEWSGPSKMIAPILDEIADEYQGKLTVAKLNIDONPGTAPKYGIRGIPTL | |
| | LLFKNGEVAATKVGALSKGOLKEFLDANLAGSAGSAGSAGSAGSAGSAGSAGSAGSAGRS | |

Italic - Trx
Underlined = linker
Double underlined = modified linker
Bold = sequence of the modification

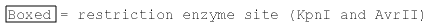

### CONCLUSION

Here, we demonstrate the nanopore detection of widely separated phosphorylation sites within a polypeptide chain by using PAZn₂, an exemplary chemical binder based on the Phos-tag. The binder created a distinct two-level current feature when phosphorylated polypeptide segments were inside the nanopore, which resembled current patterns observed during divalent cation chelation within an engineered αHL pore²¹ or with amino acids interacting with immobilized Ni²⁺ in an engineered nanopore²³. The phosphorylation-specific current feature enabled the discrimination of phosphorylation from PTMs that produced similar current blockades. As a proof of concept, we were able to distinguish glutathionylation from phosphorylation by this means. Those skilled in the art will appreciate that combinations of PTM-specific binders will allow the simultaneous detection of multiple PTMs.

Given the tight binding reported between a phosphoserine-containing peptide and the Phos-tag (K_{d}=˜ 0.7 μM)¹⁹ along with the excess PAZn₂ present (50 eq.), we presumed the detection of phosphorylated segments always in the PAZn₂-bound state under the conditions used. However, the detection probability of ˜80% indicates the possible presence of anionic species in the recording solution competing for PAZn₂ binding. For example, the sulfate-based buffering reagent, 2-[4-(2-Hydroxyethyl) piperazin-1-yl]ethane-1-sulfonic acid (HEPES), and the electrolyte, Cl-ions, might occupy the Phos-tag transiently but frequently at mM concentrations. For future profiling of phosphorylation patterns on individual polypeptides, we could either look for non-competing buffering reagents or balance the concentrations of the Phos-tag and anionic species to ensure 100% binder association when a phosphorylated residue is inside the pore. So far, we have identified PTMs in polypeptide segments while they are transiently arrested within a nanopore. To identify PTMs in domains that are freely moving, bulky binders, such as antibodies, which might temporarily halt protein translocation at the mouth of the pore could be used.

### REFERENCES FOR EXAMPLE 2

(1) Ramazi, S.; Zahiri, J. Database 2021, No. baab012.
(2) Xu, H. et al, Genom. Proteom. Bioinform. 2018, 16 (4), 244-251.
(3) Xu, S. et al, Cell Rep. 2023, 42 (7), 112796.
(4) Hu, P. et al, FEBS Lett. 2010, 2526-2538.
(5) Nurk, S. et al, Science 2022, 376 (6588), 44-53.
(6) Parker, M. T. et al, Elife 2020, 9. e49658.
(7) Rodriguez-Larrea, D.; Bayley, H. Nat. Nanotechnol. 2013, 8 (4), 288-295.
(8) Rosen, C. B. et al, Nat. Biotechnol. 2014, 32 (2), 179-181.
(9) Ying, Y. L. et al, Nat. Nanotechnol. 2022, 17 (11), 1136-1146.
(10) Restrepo-Pérez, L. et al, Nat. Nanotechnol. 2018, 13 (9), 786-796.
(11) Nivala, J. et al, Nat. Biotechnol. 2013, 31 (3), 247-250.
(12) Yu, L. et al, Nat. Biotechnol. 2023, 41 (8), 1130-1139.
(13) Martin-Baniandres, P. et al, Nat. Nanotechnol. 2023. 18, 1335-1340.
(14) Sauciuc, A. et al, Nat. Biotechnol. 2023.
(15) Restrepo-Pérez, L. et al, Nano Lett. 2019, 19 (11), 7957-7964.
(16) Ensslen, T. et al, J. Am. Chem. Soc. 2022, 144 (35), 16060-16068.
(17) Nova, I. C. et al, Nat. Biotechnol. 2023.
(18) Kinoshita, E. et al, Dalton Trans. 2004, 8, 1189-1193.
(19) Takiyama, K. et al, Anal. Biochem. 2009, 388, 235-241.
(20) Gu, L.-Q. et al, Proc. Natl. Acad. Sci. U.S.A. 2003, 100 (26), 15498-15503.
(21) Hammerstein, A. F. et al, Angew. Chem., Int. Ed. 2010, 49, 5085-5090.
(22) Ojida, A. et al, J. Am. Chem. Soc. 2004, 126 (8), 2454-2463.
(23) Wang, K. et al, Nat. Methods 2023.

### Supplementary Information for Example 2

TABLE S2
Percentage residual current (I_{res %}) and root-mean-square noise
(I_{r.m.s.}) characteristics of A1-P and A1-P-PAZn₂^{[a], [b]}

| | | | I_{r.m.s.}/ | |
|---|---|---|---|---|
| | | ΔI_{res %}^{[a]} | pA^{[b]} | N |
| A1-P | | 7.8 ± 1.7% | 1.8 ± 0.4 | 39 concatemers |
| | | | | 3 separate pores |
| A1-P- | A1-P-PAZn₂-H | 3.7 ± 2.0% | 1.4 ± 0.5 | 29 concatemers |
| PAZn₂ | A1-P-PAZn₂-L | 15 ± 1% | 1.3 ± 0.4 | 3 separate pores |

^{[a]}ΔI_{res %} = <I_{res %}(A1, Trx-linker)> − I_{res %}(A1-P), <I_{res %}(A1, Trx-linker)> − I_{res %}(A1-P-PAZn₂-H), or <I_{res %}(A1, Trx-linker)> − I_{res %}(A1-P-PAZn₂-L). For a C terminus-first translocation event, <I_{res %}(A1, Trx-linker)> was determined as the mean I_{res %} value of the unmodified A1 levels within an individual translocation event. I_{res %}(A1-P) was determined for the A1 level of the modified linker and appeared once or twice per translocating pentamer. I_{res %}(A1-P-PAZn₂-H) and I_{res %}(A1-P-PAZn₂-L) were determined for the higher and lower levels of the two-level A1-P-PAZn₂ state, which appeared once or twice per translocating pentamer. If two A1-P or A1-P-PAZn₂ were detected in a single translocation event, they were analyzed individually. Conditions: 10 mM HEPES, pH 7.2, 750 mM GdnHCl, +140 mV (trans), 23 ± 1° C.
^{[b]}Root-mean-square noise values (Ir.m.s.) were measured from current traces after a post-recording filter of 2 kHz. Ir.m.s. was normalised for the noise of each pore (I_{r.m.s.}² = I_{r.m.s.}(A1-P)² − Ir.m.s.(open pore)², I_{r.m.s.}² = I_{r.m.s.}(A1-P-PAZn₂-H)² − I_{r.m.s.}(open pore)², I_{r.m.s.}² = I_{r.m.s.}(A1-P-PAZn₂-L)² − I_{r.m.s.}(open pore)²).

TABLE S3
Mean dwell times (<τ>) derived by QuB^{[a]} for two-
level A1-P-PAZn₂^{[b]}

| Voltage (trans) | +140 mV | |
|---|---|---|
| <τ_{A1-P-PAZn2-H}>/ms | 3.3 ± 0.1 | N = 224 |
| <τ_{A1-P-PAZn2-L}>/ms | 11.6 ± 0.3 | N = 234 |

^{[a]}Dwell time analysis was performed by using the maximum interval likelihood algorithm of QuB.^{1, 2}
^{[b]}Conditions: 10 mM HEPES, pH 7.2, 750 mM GdnHCl, 2.37 μM Trx-linker pentamer (cis), 118.5 μM Phos-tag-acrylamide (cis), 237 μM ZnCl₂ (cis), +140 mV (trans), 23 ± 1° C. The C terminus-first translocations were recorded for Trx-linker pentamers through a single (NN-113R)₇ nanopore

FIG. 17 shows fractions of phosphorylated linkers detected in the PAZn₂-bound state. The fractions of events containing at least one level A1-P-PAZn₂ were tested in two molar equivalents of Phos-tag-acrylamide dizinc complexes (10 eq. and 50 eq.) against the doubly phosphorylated Trx-linker pentamer.

Fractions (%) of events containing at least one level A1-P-PAZn₂ were calculated as:

$$Percentage = \frac{events containing at least one level A 1 - P - {PAZn}_{2}}{\begin{matrix}events containing at least one level A 1 - P - {PAZn}_{2} + \\ events containing only level A 1 - P\end{matrix}}$$

where a translocation event for a phosphorylated Trx-linker concatemer was characterized by observing a minimum of one instance of level A1-P-PAZn₂ or level A1-P. If a single translocation exhibited both level A1-P-PAZn₂ and level A1-P in two distinct modified segments, it was counted as an event containing at least one level A1-P-PAZn₂. Conditions in 10×: 10 mM HEPES, pH 7.2, 750 mM GdnHCl, 2.37 μM Trx-linker pentamer (cis), 23.7 UM Phos-tag-acrylamide (cis), 47.4 μM ZnCl₂ (cis), +140 mV (trans), 23±1° C. Conditions in 50×: 10 mM HEPES, pH 7.2, 750 mM GdnHCl, 2.37 μM Trx-linker pentamer (cis), 118.5 μM Phos-tag-acrylamide (cis), 237 μM ZnCl₂ (cis), +140 mV (trans), 23±1° C.

FIG. 18 shows fractions of phosphorylated linkers detected in the PZn₂-bound state. The fractions of events containing at least one level A1-P-PZn₂ were tested in 100 and 1000 molar equivalents of Phos-tag dizinc complexes (100× and 1000×) against the doubly phosphorylated Trx-linker pentamer.

Fractions (%) of events containing at least one level A1-P-PZn₂ were calculated as:

$$Percentage = \frac{events containing at least one level A 1 - P - {PZn}_{2}}{\begin{matrix}events containing at least one level A 1 - P - {PZn}_{2} + \\ events containing only level A 1 - P\end{matrix}}$$

where a translocation event for a phosphorylated Trx-linker concatemer was characterized by observing a minimum of one instance of level A1-P-PZn₂ or level A1-P. If a single translocation exhibited both level A1-P-PZn₂ and level A1-P in two distinct modified segments, it was counted as an event containing at least one level A1-P-PZn₂. Conditions in 100×: 10 mM HEPES, pH 7.2, 750 mM GdnHCl, 2.37 μM Trx-linker pentamer (cis), 237 μM Phos-tag (cis), 474 μM ZnCl₂ (cis), +140 mV (trans), 23±1° C. Conditions in 1000×: 10 mM HEPES, pH 7.2, 750 mM GdnHCl, 2.37 μM Trx-linker pentamer (cis), 2.37 mM Phos-tag (cis), 4.74 mM ZnCl₂ (cis), +140 mV (trans), 23±1° C.

FIG. 19 shows fractions of events containing at least one level A1-P-PAZn₂ in the absence and presence of competing phosphoserine. Before pSer addition, 79% of the translocation events with a minimum of one phosphorylated linker detected either in the PAZn2-bound or unbound state (29 events) showed at least one level A1-P-PAZn₂. After pSer addition, 16% of the translocation events with a minimum of one phosphorylated linker detected either in the PAZn₂-bound or unbound state (24 events) showed at least one level A1-P-PAZn₂. Conditions before adding pSer: 10 mM HEPES, pH 7.2, 750 mM GdnHCl, 2.37 μM Trx-linker pentamer (cis), 118.5 μM Phos-tag-acrylamide (cis), 237 μM ZnCl₂ (cis), +140 mV (trans), 23±1° C. Conditions after adding pSer: 10 mM HEPES, pH 7.2, 750 mM GdnHCl, 2.37 μM Trx-linker pentamer (cis), 118.5 μM Phos-tag-acrylamide (cis), 237 μM ZnCl2 (cis), 237 μM pSer (cis), +140 mV (trans), 23±1° C.

FIG. 20 shows a current trace showing transition between level A1-P-PAZn₂ and level A1-P when a phosphorylated segment was inside the (NN-113R) 7 nanopore. Conditions: 10 mM HEPES, pH 7.2, 750 mM GdnHCl, 2.37 μM Trx-linker pentamer (cis), 118.5 μM Phos-tag-acrylamide (cis), 237 μM ZnCl₂ (cis), +140 mV (trans), 23±1° C.

### Methods

Construction of his-SUMO-Tagged Trx-Linker Pentamer Genes

Reagents were purchased from NEB (New England Biolabs), unless otherwise stated. His-SUMO-tagged Trx-linker pentamer genes were prepared as previously described^{3,4}. Two variants of His-SUMO-tagged Trx-linker pentamers were prepared to contain two phosphorylation sites within the second and fourth linkers (His-SUMO-tagged (Trx-linker)_{1,3,5} (Trx-linker-24S26C)_{2,4}) or one phosphorylation site within the second linker and one glutathionylation site within the fourth linker (His-SUMO-tagged (Trx-linker)_{1,3,5} (Trx-linker-24S)₂ (Trx-linker-26C)₄).

### Expression and Purification of Trx-Linker Pentamers

Plasmids encoding the Trx-linker pentamer were transformed into BLR (DE3) competent cells (Novagen), which were cultivated in Luria broth (LB) supplemented with carbenicillin (100 μg/mL) at 37° C. with constant agitation at 250 rpm. Protein expression was induced in the exponential growth phase (OD₆₀₀=0.6 to 0.8) by adding isopropyl-β-D-1-thiogalactopyranoside (IPTG) to a final concentration of 0.5 mM. After 6 h, cells were harvested by centrifugation (at 5,000 g for 10 minutes), resuspended in a binding buffer (containing 30 mM Tris-HCl, 250 mM NaCl, 25 mM imidazole, pH 7.2) supplemented with a protease inhibitor cocktail (cOmplete™, EDTA-free, Roche), and lysed by sonication. Cell debris was removed by centrifugation at 20,000 g for 40 min, and the supernatant was loaded onto a column packed with HisPur Ni-NTA Agarose Resin (5 mL, ThermoFisher) equilibrated with binding buffer (25 mM Tris-HCl, pH 7.5, 500 mM NaCl, 25 mM imidazole) and the flow through was re-applied 5 times. After washing with binding buffer, the hexahistidine (His6)-tagged protein was eluted with 12 mL elution buffer (25 mM Tris-HCl, pH 7.5, 500 mM NaCl, 500 mM imidazole) and dialysed (Slide-A-Lyzer G2 Dialysis Cassette, 10,000 MWCO 30 mL, ThermoFisher) for 2 h against 4 L of dialysis buffer (50 mM Tris-HCl, pH 8.0, 150 mM NaCl, 2 mM 1,4-dithio-D-threitol (DTT)), with continuous stirring at 4° C., to remove imidazole. Then, His6-tagged Ulp1 protease, prepared as previously described⁴, was injected into the dialysis cassette at a 1:200 molar concentration ratio with respect to the Trx-linker pentamer. Afterwards, the cassette was transferred to DTT-free dialysis buffer (50 mM Tris-HCl, pH 8.0, 150 mM NaCl) overnight for SUMO-tag cleavage. The cassette was then transferred to DTT-free dialysis buffer for an additional 4 h. The dialysed protein was loaded onto a column packed with 5 mL HisPur Ni-NTA Agarose Resin equilibrated with dialysis buffer and the flow through was re-applied to the column 5 more times. The final flow through containing the His6-SUMO-free protein was aliquoted and flash frozen for storage at −80° C. The mass of the protein was confirmed by electrospray ionization liquid chromatography-mass spectrometry (ESI LCMS) (FIG. 16).

### Phosphorylation of Trx-Linker Pentamers

Trx-linker pentamers containing two phosphorylation sites within the second and fourth linkers or a single phosphorylation site within the second linker were phosphorylated by the catalytic subunit of the cAMP-dependent protein kinase (PKA) (NEB). The Trx-linker pentamers at a concentration of 1 mg/mL were incubated with 25,000 units of cAMP-dependent protein kinase (PKA) catalytic subunit (NEB), which phosphorylates the RRAS motif on serine. The buffer used contained 50 mM TrisHCl, pH 7.5.10 mM MgCl2, 0.1 mM EDTA, 4 mM DTT, 0.01% Brij 35, and 2 mM ATP at 30° C. for 1 h. Then, the mixture was further supplemented with an additional 2 mM ATP and 2 mM DTT, followed by incubation at 30° C. for one more hour. The phosphorylated Trx-linker pentamers were purified and concentrated by using centrifugal filters (Vivaspin 2 centrifugal concentrators MWCO 50 kDa). They were then aliquoted and flash frozen for storage at −20° C. (10 mM HEPES, pH 7.2, and 750 mM KCl). Phosphorylation of the Trx-linker pentamers was verified by LCMS (FIG. 16).

### Modification of Cysteine on Trx-Linker Pentamers

Trx-linker pentamers containing a phosphorylation site within the second linker and a glutathionylation site within the fourth linker were first phosphorylated following the steps described in the above section. To subsequently glutathionylate the singly phosphorylated Trx-linker pentamers, they were treated with tris(2-carboxyethyl) phosphine (TCEP, Sigma-Aldrich) (100 eq.) at 32° C. for 2 h in protein storage buffer (50 mM TrisHCl, 250 mM NaCl, pH 8.0) and then desalted with PD MiniTrap G-25 columns (Cytiva). The reduced proteins were reacted with oxidized glutathione (100 eq.) (Sigma-Aldrich) at 32° C. overnight in protein storage buffer before desalting (PD MiniTrap G-25 columns). The glutathionylated proteins were aliquoted, flash frozen, and stored at −20° C.

### Phosphoserine Competition Assay

The phosphorylated Trx-linker pentamer was mixed with Phos-tag-acrylamide dizinc complex with a molar ratio of Trx-linker:Phos-tag-acrylamide:ZnCl2=1:50:100 and kept at room temperature for 15 min. The mixture was then added to the cis compartment of the recording chamber (final concentrations in the cis compartment: 2.37 μM Trx-linker pentamers, 118.5 μM Phos-tag-acrylamide, 237 μM ZnCl2, 10 mM HEPES, pH 7.2, 750 mM GdnHCl). After recording for ˜10 min, phosphoserine was introduced to the same compartment to a final concentration of 237 μM and another 10-min recording was performed.

Fractions (%) of events containing at least one level A1-P-PAZn2 were calculated as:

$$Percentage = \frac{events containing at least one level A 1 - P - {PAZn}_{2}}{\begin{matrix}events containing at least one level A 1 - P - {PAZn}_{2} + \\ events containing only level A 1 - P\end{matrix}}$$

where a translocation event for a phosphorylated Trx-linker concatemer was characterized by observing a minimum of one instance of level A1-P-PAZn2 or level A1-P. If a single translocation exhibited both level A1-P-PAZn2 and level A1-P in two distinct modified segments, it was counted as an event containing at least one level A1-P-PAZn2.

### Single-Channel Recording

Electrical recordings were performed with planar lipid bilayers at 23.0±1.0° C. Planar bilayers composed of 1,2-diphytanoyl-sn-glycero-3-phosphocholine (Avanti Polar Lipids) were formed by using the Müller-Montal method across a 50 μm-diameter aperture in a Teflon film (25 μm thick, Goodfellow) separating the cis and trans compartments of the recording chamber (500 μL each). Each compartment was filled with 500 μL recording buffer (10 mM HEPES, pH 7.2, 750 mM GdnHCl). Following the insertion of a single pore into the bilayer, the solution was perfused by manual pipetting to prevent further insertions. Trx-linker pentamers or Trx-linker pentamers with Phos-tag dizinc complex were added to the cis compartment (Trx-linker pentamers, 2.37 μM; Phos-tag-acrylamide, 118.5 μM; ZnCl2, 237 μM). For experiments in the presence of Phos-tag-acrylamide, the phosphorylated Trx-linker pentamer was incubated with Phos-tag-acrylamide dizinc complex at room temperature for 15 min. Then the mixture was added to the cis compartment (Trx-linker pentamers, 2.37 μM; Phos-tag-acrylamide, 118.5 μM; ZnCl2, 237 μM). Ionic currents were measured using Ag/AgCl electrodes connected to a patch-clamp amplifier (Axopatch 200B, Axon Instruments). Data were low-pass Bessel filtered at 10 kHz and sampled at 50 kHz with a Digidata 1440A digitizer (Molecular Devices). Current traces were idealized by using Clampfit 10.7 (Molecular Devices). Dwell time analysis for the idealized data was performed by using the maximum interval likelihood algorithm of QuB 2.0 software (www.qub.buffalo.edu)^{1,2}.

### SUPPLEMENTARY REFERENCES FOR EXAMPLE 2

(1) Qin, F. et al, Biophys. J. 1996, 70, 264-280.
(2) Nicolai, C.; Sachs, F. Biophys. Rev. 6 Lett. 2013, 08, 191-211.
(3) Carrion-Vazquez, M. et al, Proc. Natl. Acad. Sci. U.S.A. 1999, 96, 3694-3699
(4) Martin-Baniandres, P. et al, Nat. Nanotechnol. 2023.18, 1335-1340.

SEQUENCE LISTING

SEQ ID NO: 1 shows the amino acid sequence of a monomer of the WT aHL nanopore.
SEQ ID NO: 2 shows the amino acid sequence of a monomer of the aHL-NN-113R
nanopore used in the examples.
SEQ ID NOs: 3 to 8 show the amino acid sequence of Trx concatamers used in the
examples.
SEQ ID NO: 9 shows the amino acid sequence of a protein linker used in the construction
of Trx concatamers used in the examples.
SEQ ID NOs: 10-18 denote sequences disclosed herein.
SEQ ID NO: 19 shows the amino acid sequence of thioredoxin-linker pentamers described
in Example 2 (see Table 7).
SEQ ID NO: 20 shows the amino acid sequence of thioredoxin-linker pentamers described
in Example 2 (see Table 7).
SEQ ID NOs: 21-24 relate to sequences shown in Figure 12 and SEQ ID NOs: 25-26 relate
to sequences shown in FIG.14.
SEQ ID NO: 1
ADSDINIKTGTTDIGSNTTVKTGDLVTYDKENGMHKKVFYSFIDDKNHNKKLLVIRTKGTIAGQYR
VYSEEGANKSGLAWPSAFKVQLQLPDNEVAQISDYYPRNSIDTKEYMSTLTYGFNGNVTGDDTGKI
GGLIGANVSIGHTLKYVQPDFKTILESPTDKKVGWKVIFNNMVNQNWGPYDRDSWNPVYGNQLFMK
TRNGSMKAADNFLDPNKASSLLSSGFSPDFATVITMDRKASKQQTNIDVIYERVRDDYQLHWTSTN
WKGTNTKDKWTDRSSERYKIDWEKEEMTN
SEQ ID NO: 2
ADSDINIKTGTTDIGSNTTVKTGDLVTYDKENGMHKKVFYSFIDDKNHNKKLLVIRTKGTIAGQYR
VYSEEGANKSGLAWPSAFKVQLQLPDNEVAQISDYYPRNSIDTKNYRSTLTYGFNGNVTGDDTGKI
GGLIGANVSIGHTLNYVQPDFKTILESPTDKKVGWKVIFNNMVNQNWGPYDRDSWNPVYGNQLFMK
TRNGSMKAADNFLDPNKASSLLSSGFSPDFATVITMDRKASKQQTNIDVIYERVRDDYQLHWTSTN
WKGTNTKDKWTDRSSERYKIDWEKEEMTN
SEQ ID NO: 3
CGSDKIIHLTDDSFDTDVLKADGAILVDFWAEWSGPSKMIAPILDEIADEYQGKLTVAKLNIDQNP
GTAPKYGIRGIPTLLLFKNGEVAATKVGALSKGQLKEFLDANLAGSAGSAGSAGSAGSAGSAGSAG
SAGSAGRSDKIIHLTDDSFDTDVLKADGAILVDFWAEWSGPSKMIAPILDEIADEYQGKLTVAKLN
IDQNPGTAPKYGIRGIPTLLLFKNGEVAATKVGALSKGQLKEFLDANLAGSAGSAGSAGSAGSAGS
AGSAGSAGSAGRS
SEQ ID NO: 4
CGSDKIIHLTDDSFDTDVLKADGAILVDFWAEWSGPSKMIAPILDEIADEYQGKLTVAKLNIDQNP
GTAPKYGIRGIPTLLLFKNGEVAATKVGALSKGQLKEFLDANLAGSAGSAGSAGSAGSAGSAGSAG
SAGSAGRSDKIIHLTDDSFDTDVLKADGAILVDFWAEWSGPSKMIAPILDEIADEYQGKLTVAKLN
IDQNPGTAPKYGIRGIPTLLLFKNGEVAATKVGALSKGQLKEFLDANLAGSAGSAGSAGSAGSAGS
AGSAGSAGSAGRSDKIIHLTDDSFDTDVLKADGAILVDFWAEWSGPSKMIAPILDEIADEYQGKLT
VAKLNIDQNPGTAPKYGIRGIPTLLLFKNGEVAATKVGALSKGQLKEFLDANLAGSAGSAGSAGSA
GSAGSAGSAGSAGSAGRSDKIIHLTDDSFDTDVLKADGAILVDFWAEWSGPSKMIAPILDEIADEY
QGKLTVAKLNIDQNPGTAPKYGIRGIPTLLLFKNGEVAATKVGALSKGQLKEFLDANLAGSAGSAG
SAGSAGSAGSAGSAGSAGSAGRS
SEQ ID NO: 5
CGSDKIIHLTDDSFDTDVLKADGAILVDFWAEWSGPSKMIAPILDEIADEYQGKLTVAKLNIDQNP
GTAPKYGIRGIPTLLLFKNGEVAATKVGALSKGQLKEFLDANLAGSAGSAGSAGSAGSAGSAGSAG
SAGSAGRSDKIIHLTDDSFDTDVLKADGAILVDFWAEWSGPSKMIAPILDEIADEYQGKLTVAKLN
IDQNPGTAPKYGIRGIPTLLLFKNGEVAATKVGALSKGQLKEFLDANLAGSAGSAGSAGSAGSAGS
AGSAGSAGSAGRSDKIIHLTDDSFDTDVLKADGAILVDFWAEWSGPSKMIAPILDEIADEYQGKLT
VAKLNIDQNPGTAPKYGIRGIPTLLLFKNGEVAATKVGALSKGQLKEFLDANLAGSAGSAGSAGSA
GSAGSAGSAGSAGSAGRSDKIIHLTDDSFDTDVLKADGAILVDFWAEWSGPSKMIAPILDEIADEY
QGKLTVAKLNIDQNPGTAPKYGIRGIPTLLLFKNGEVAATKVGALSKGQLKEFLDANLAGSAGSAG
SAGSAGSAGSAGSAGSAGSAGRSDKIIHLTDDSFDTDVLKADGAILVDFWAEWSGPSKMIAPILDE
IADEYQGKLTVAKLNIDQNPGTAPKYGIRGIPTLLLFKNGEVAATKVGALSKGQLKEFLDANLAGS
AGSAGSAGSAGSAGSAGSAGSAGSAGRSDKIIHLTDDSFDTDVLKADGAILVDFWAEWSGPSKMIA
PILDEIADEYQGKLTVAKLNIDQNPGTAPKYGIRGIPTLLLFKNGEVAATKVGALSKGQLKEFLDA
NLAGSAGSAGSAGSAGSAGSAGSAGSAGSAGRS
SEQ ID NO: 6
CGSDKIIHLTDDSFDTDVLKADGAILVDFWAEWSGPSKMIAPILDEIADEYQGKLTVAKLNIDQNP
GTAPKYGIRGIPTLLLFKNGEVAATKVGALSKGQLKEFLDANLAGSAGSAGSAGSAGSAGSAGSAG
SAGSAGRSDKIIHLTDDSFDTDVLKADGAILVDFWAEWSGPSKMIAPILDEIADEYQGKLTVAKLN
IDQNPGTAPKYGIRGIPTLLLFKNGEVAATKVGALSKGQLKEFLDANLAGSAGSAGSAGSAGSAGS
AGSAGSAGSAGRSDKIIHLTDDSFDTDVLKADGAILVDEWAEWSGPSKMIAPILDEIADEYQGKLT
VAKLNIDQNPGTAPKYGIRGIPTLLLFKNGEVAATKVGALSKGQLKEFLDANLAGSAGSAGSAGSA
GSAGSAGSAGSAGSAGRSDKIIHLTDDSFDTDVLKADGAILVDFWAEWSGPSKMIAPILDEIADEY
QGKLTVAKLNIDQNPGTAPKYGIRGIPTLLLFKNGEVAATKVGALSKGQLKEFLDANLAGSAGSAG
SAGSAGSAGSAGSAGSAGSAGRSDKIIHLTDDSFDTDVLKADGAILVDFWAEWSGPSKMIAPILDE
IADEYQGKLTVAKLNIDQNPGTAPKYGIRGIPTLLLFKNGEVAATKVGALSKGQLKEFLDANLAGS
AGSAGSAGSAGSAGSAGSAGSAGSAGRSDKIIHLTDDSFDTDVLKADGAILVDFWAEWSGPSKMIA
PILDEIADEYQGKLTVAKLNIDQNPGTAPKYGIRGIPTLLLFKNGEVAATKVGALSKGQLKEFLDA
NLAGSAGSAGSAGSAGSAGSAGSAGSAGSAGRSDKIIHLTDDSFDTDVLKADGAILVDFWAEWSGP
SKMIAPILDEIADEYQGKLTVAKLNIDQNPGTAPKYGIRGIPTLLLFKNGEVAATKVGALSKGQLK
EFLDANLAGSAGSAGSAGSAGSAGSAGSAGSAGSAGRSDKIIHLTDDSFDTDVLKADGAILVDFWA
EWSGPSKMIAPILDEIADEYQGKLTVAKLNIDQNPGTAPKYGIRGIPTLLLFKNGEVAATKVGALS
KGQLKEFLDANLA
SEQ ID NO: 7
SDKIIHLTDDSFDTDVLKADGAILVDFWAEWSGPSKMIAPILDEIADEYQGKLTVAKLNIDQNPGT
APKYGIRGIPTLLLFKNGEVAATKVGALSKGQLKEFLDANLAGSAGSAGSAGSAGSAGSAGSAGSA
GSAGRSDKIIHLTDDSFDTDVLKADGAILVDFWAEWSGPSKMIAPILDEIADEYQGKLTVAKLNID
QNPGTAPKYGIRGIPTLLLFKNGEVAATKVGALSKGQLKEFLDANLAGSAGSAGSAGSAGSAGSAG
SAGSAGSAGRSDKIIHLTDDSFDTDVLKADGAILVDFWAEWSGPSKMIAPILDEIADEYQGKLTVA
KLNIDQNPGTAPKYGIRGIPTLLLFKNGEVAATKVGALSKGQLKEFLDANLAGSAGSAGSAGSAGS
AGSAGSAGSAGSAGRSDKIIHLTDDSFDTDVLKADGAILVDFWAEWSGPSKMIAPILDEIADEYQG
KLTVAKLNIDQNPGTAPKYGIRGIPTLLLFKNGEVAATKVGALSKGQLKEFLDANLAGSAGSAGSA
GSAGSAGSAGSAGSAGSAGRSGTSDKIIHLTDDSFDTDVLKADGAILVDFWAEWSGPSKMIAPILD
EIADEYQGKLTVAKLNIDQNPGTAPKYGIRGIPTLLLFKNGEVAATKVGALSKGQLKEFLDANLAG
SAGSAGSAGSAGSAGSAGSRRASACAGSAGSAGRSPRRSDKIIHLTDDSFDTDVLKADGAILVDFW
AEWSGPSKMIAPILDEIADEYQGKLTVAKLNIDQNPGTAPKYGIRGIPTLLLFKNGEVAATKVGAL
SKGQLKEFLDANLAGSAGSAGSAGSAGSAGSAGSAGSAGSAGRSDKIIHLTDDSFDTDVLKADGAI
LVDFWAEWSGPSKMIAPILDEIADEYQGKLTVAKLNIDQNPGTAPKYGIRGIPTLLLFKNGEVAAT
KVGALSKGQLKEFLDANLAGSAGSAGSAGSAGSAGSAGSAGSAGSAGRSDKIIHLTDDSFDTDVLK
ADGAILVDFWAEWSGPSKMIAPILDEIADEYQGKLTVAKLNIDQNPGTAPKYGIRGIPTLLLFKNG
EVAATKVGALSKGQLKEFLDANLAGSAGSAGSAGSAGSAGSAGSAGSAGSAGRSDKIIHLTDDSFD
TDVLKADGAILVDFWAEWSGPSKMIAPILDEIADEYQGKLTVAKLNIDQNPGTAPKYGIRGIPTLL
LFKNGEVAATKVGALSKGQLKEFLDANLAGSAGSAGSAGSAGSAGSAGSAGSAGSAGRS
SEQ ID NO: 8
SDKIIHLTDDSFDTDVLKADGAILVDFWAEWSGPSKMIAPILDEIADEYQGKLTVAKLNIDQNPGT
APKYGIRGIPTLLLFKNGEVAATKVGALSKGQLKEFLDANLAGSAGSAGSAGSAGSAGSAGSAGSA
GSAGRSDKIIHLTDDSFDTDVLKADGAILVDFWAEWSGPSKMIAPILDEIADEYQGKLTVAKLNID
QNPGTAPKYGIRGIPTLLLFKNGEVAATKVGALSKGQLKEFLDANLAGSAGSAGSAGSAGSAGSAG
SAGSAGSAGRSDKIIHLTDDSFDTDVLKADGAILVDFWAEWSGPSKMIAPILDEIADEYQGKLTVA
KLNIDQNPGTAPKYGIRGIPTLLLFKNGEVAATKVGALSKGQLKEFLDANLAGSAGSAGSAGSAGS
AGSAGSAGSAGSAGRSDKIIHLTDDSFDTDVLKADGAILVDFWAEWSGPSKMIAPILDEIADEYQG
KLTVAKLNIDQNPGTAPKYGIRGIPTLLLFKNGEVAATKVGALSKGQLKEFLDANLAGSAGSAGSA
GSAGSAGSAGSAGSAGSAGRSGTSDKIIHLTDDSFDTDVLKADGAILVDFWAEWSGPSKMIAPILD
EIADEYQGKLTVAKLNIDQNPGTAPKYGIRGIPTLLLFKNGEVAATKVGALSKGQLKEFLDANLAG
SAGSAGSAGRRASACSAGSAGSAGSAGSAGSAGRSPRRSDKIIHLTDDSFDTDVLKADGAILVDFW
AEWSGPSKMIAPILDEIADEYQGKLTVAKLNIDQNPGTAPKYGIRGIPTLLLFKNGEVAATKVGAL
SKGQLKEFLDANLAGSAGSAGSAGSAGSAGSAGSAGSAGSAGRSDKIIHLTDDSFDTDVLKADGAI
LVDFWAEWSGPSKMIAPILDEIADEYQGKLTVAKLNIDQNPGTAPKYGIRGIPTLLLFKNGEVAAT
KVGALSKGQLKEFLDANLAGSAGSAGSAGSAGSAGSAGSAGSAGSAGRSDKIIHLTDDSFDTDVLK
ADGAILVDFWAEWSGPSKMIAPILDEIADEYQGKLTVAKLNIDQNPGTAPKYGIRGIPTLLLFKNG
EVAATKVGALSKGQLKEFLDANLAGSAGSAGSAGSAGSAGSAGSAGSAGSAGRSDKIIHLTDDSFD
TDVLKADGAILVDFWAEWSGPSKMIAPILDEIADEYQGKLTVAKLNIDQNPGTAPKYGIRGIPTLL
LFKNGEVAATKVGALSKGQLKEFLDANLAGSAGSAGSAGSAGSAGSAGSAGSAGSAGRS
SEQ ID NO: 9
GSAGSAGSAGSAGSAGSAGSAGSAGSAGR
SEQ ID NOs: 10-18-see text
SEQ ID NO: 19
SDKIIHLTDDSFDTDVLKADGAILVDFWAEWSGPSKMIAPILDEIADEYQGKLTVAKLNIDQNPGT
APKYGIRGIPTLLLFKNGEVAATKVGALSKGQLKEFLDANLAGSAGSAGSAGSAGSAGSAGSAG
SAGSAGRSDKIIHLTDDSFDTDVLKADGAILVDFWAEWSGPSKMIAPILDEIADEYQGKLTVAKLN
IDQNPGTAPKYGIRGIPTLLLFKNGEVAATKVGALSKGQLKEFLDANLAGSAGSAGSAGSAGSA
GSAGSRRASACAGSAGSAGRSDKIIHLTDDSFDTDVLKADGAILVDFWAEWSGPSKMIAPILDEI
ADEYQGKLTVAKLNIDQNPGTAPKYGIRGIPTLLLFKNGEVAATKVGALSKGQLKEFLDANLAGS
AGSAGSAGSAGSAGSAGSAGSAGSAGRSGTGGPRRRSDKIIHLTDDSFDTDVLKADGAILVDF
WAEWSGPSKMIAPILDEIADEYQGKLTVAKLNIDQNPGTAPKYGIRGIPTLLLFKNGEVAATKVG
ALSKGQLKEFLDANLAGSAGSAGSAGSAGSAGSAGSRRASACAGSAGSAGRSDKIIHLTDDSF
DTDVLKADGAILVDFWAEWSGPSKMIAPILDEIADEYQGKLTVAKLNIDQNPGTAPKYGIRGIPTL
LLFKNGEVAATKVGALSKGQLKEFLDANLAGSAGSAGSAGSAGSAGSAGSAGSAGSAGRS
SEQ ID NO: 20
SDKIIHLTDDSFDTDVLKADGAILVDEWAEWSGPSKMIAPILDEIADEYQGKLTVAKLNIDQNPGT
APKYGIRGIPTLLLFKNGEVAATKVGALSKGQLKEFLDANLAGSAGSAGSAGSAGSAGSAGSAG
SAGSAGRSDKIIHLTDDSFDTDVLKADGAILVDFWAEWSGPSKMIAPILDEIADEYQGKLTVAKLN
IDQNPGTAPKYGIRGIPTLLLFKNGEVAATKVGALSKGQLKEFLDANLAGSAGSAGSAGSAGSA
GSAGSRRASAAAGSAGSAGRSDKIIHLTDDSFDTDVLKADGAILVDFWAEWSGPSKMIAPILDEI
ADEYQGKLTVAKLNIDQNPGTAPKYGIRGIPTLLLFKNGEVAATKVGALSKGQLKEFLDANLAGS
AGSAGSAGSAGSAGSAGSAGSAGSAGRSGTGGPRRRSDKIIHLTDDSFDTDVLKADGAILVDF
WAEWSGPSKMIAPILDEIADEYQGKLTVAKLNIDQNPGTAPKYGIRGIPTLLLFKNGEVAATKVG
ALSKGQLKEFLDANLAGSAGSAGSAGSAGSAGSAGSRRAAACAGSAGSAGRSDKIIHLTDDSF
DTDVLKADGAILVDEWAEWSGPSKMIAPILDEIADEYQGKLTVAKLNIDQNPGTAPKYGIRGIPTL
LLFKNGEVAATKVGALSKGQLKEFLDANLAGSAGSAGSAGSAGSAGSAGSAGSAGSAGRS

1. A method of characterising a peptide, polypeptide or protein at least 25 amino acids in length; comprising
contacting the peptide, polypeptide or protein with an engineered protein nanopore having a first opening, a second opening and a solvent-accessible channel therebetween;
under conditions such that an electroosmotic force across the nanopore causes the peptide, polypeptide or protein to translocate through the nanopore in a linearised state; and
taking one or more measurements characteristic of the peptide, polypeptide or protein as the peptide, polypeptide or protein translocates the nanopore;
thereby characterising the peptide, polypeptide or protein.

2. A method according to claim 1, wherein said method is a method of characterising one or more proteoforms of said peptide, polypeptide or protein.

3. A method of characterising one or more proteoforms of a peptide, polypeptide or protein; comprising
contacting the peptide, polypeptide or protein with a nanopore under conditions such that an electroosmotic force across the nanopore causes the peptide, polypeptide or protein to translocate through the nanopore in a linearised state; and
taking one or more measurements characteristic of the peptide, polypeptide or protein as the peptide, polypeptide or protein translocates the nanopore;
thereby characterising the proteoforms of the peptide, polypeptide or protein.

4. A method according to claim 3, wherein said nanopore is a engineered protein nanopore having a first opening, a second opening and a solvent-accessible channel therebetween.

5. A method according to any one of the preceding claims, wherein the nanopore is a mutant protein nanopore and wherein the channel of said nanopore comprises one or more non-native charged moieties.

6. A method according to any one of claims 3 to 5, wherein said peptide, polypeptide or protein is at least 25 amino acids in length.

7. A method according to any one of claims 2 to 6, wherein said proteoforms of said peptide, polypeptide or protein that are characterised are selected from proteoforms corresponding to modifications in the genome, modifications in the RNA, modifications during translation and modifications at the protein level; somatic mutations, long-range genome rearrangements; recombinations (e.g. V(D)J recombinations), somatic hypermutations, alternative splicings, RNA base editing modifications, frameshift modifications, codon reassignments, translational bypass modifications, translational errors, modifications arising from proteolytic processing, protein splicing modifications, post-translational modifications (PTMs) and chemical rearrangements.

8. A method according to any one of claims 2 to 7, wherein characterising said proteoforms comprises detecting and/or characterising one or more post-translational modifications and/or one or more RNA splicing sites in said peptide, polypeptide or protein.

9. A method according to claim 7 or 8, wherein said method is a method of determining the presence, absence, number, position, or identity of one or more post-translational modifications at one or more sites within the peptide, polypeptide or protein; and wherein said one or more sites are preferably at least 25 amino acids from the N-terminus and/or at least 25 amino acids from the C terminus of said peptide, polypeptide or protein.

10. A method according to any one of claims 2 to 9, wherein characterising said proteoforms comprises detecting and/or characterising, preferably by determining the presence, absence, number, position, or identity, of two or more post-translational modifications.

11. A method according to claim 10, wherein said two or more post-translational modifications are separated in said peptide, polypeptide or protein by at least 50, at least 100, at least 150 or at least 200 amino acids.

12. A method according to any one of claims 1, 2, or 4 to 11, wherein the nanopore is modified to increase the ion selectivity of the nanopore;
preferably wherein the channel of the nanopore comprises one or more non-native charged moieties having a charged side chain;
more preferably wherein the one or more non-native charged moieties comprise one or more positively charged amino acids and said one or more positively charged amino acids increase the anion selectivity of the nanopore.

13. A method according to any one of the preceding claims, wherein said nanopore is a transmembrane β-barrel protein nanopore.

14. A method according to any one of the preceding claims, wherein said peptide, polypeptide or protein has a net charge of between about −10 and about +10 per 50 amino acids; preferably wherein said peptide, polypeptide or protein has a net charge of between about −5 and about +5 per 30 amino acids.

15. A method according to any one of the preceding claims, comprising contacting the peptide, polypeptide or protein with a chaotropic agent prior to the translocation of the peptide, polypeptide or protein through the nanopore and/or wherein said method is carried out in the presence of a chaotropic agent;
preferably wherein said chaotropic agent is a denaturant, more preferably wherein said chaotropic agent is selected from guanidinium salts, guanidinium isothiocyanate, urea and thiourea.

16. A method according to any one of the preceding claims, wherein said method is conducted between about pH 4 and about pH 10.

17. A method according to any one of the preceding claims, wherein said method comprises applying a voltage during said method, and wherein the voltage applied varies during the method;
preferably wherein the method comprises applying a voltage ramp during the method.

18. A method according to any one of the preceding claims, wherein said peptide, polypeptide or protein comprises a concatamer of two or more peptides, polypeptides and/or proteins;
preferably wherein the peptides, polypeptides and/or proteins in said concatamer are attached together by one or more linkers.

19. A method according to any one of the preceding claims, wherein said peptide, polypeptide or protein comprises or consists of a complete intact protein.

20. A method according to any one of the preceding claims, comprising characterising a plurality of peptides, polypeptides or proteins.

21. A method according to any one of the preceding claims, wherein:
i) the peptide, polypeptide or protein is not attached to a charged leader;
preferably wherein the peptide, polypeptide or protein is not attached to (a) a polynucleotide leader or (b) an anionic peptide such as a poly-aspartate, poly-glutamate or poly(aspartate/glutamate) leader; and/or
ii) a motor protein is not used to control the translocation of the peptide, polypeptide or protein through the nanopore.

22. A method according to any one of the preceding claims, wherein characterising said polypeptide or said proteoforms of said peptide, polypeptide or protein comprises detecting the number, position and/or nature of modifications in said peptide, polypeptide or protein as the peptide, polypeptide or protein translocates through the nanopore.

23. A method according to any one of the preceding claims, which is a method of characterising one or more post-translational modifications in a peptide, polypeptide or protein; comprising
contacting the peptide, polypeptide or protein with a label capable of binding to said one or more post-translational modifications;
contacting the peptide, polypeptide or protein with a nanopore under conditions such that an electroosmotic force across the nanopore causes the peptide, polypeptide or protein to translocate through the nanopore in a linearised state; and
taking one or more measurements characteristic of the label as the peptide, polypeptide or protein translocates the nanopore;
thereby characterising the one or more post-translational modifications of the peptide, polypeptide or protein.

24. A system, comprising
an engineered protein nanopore having a first opening, a second opening and a solvent-accessible channel therebetween;
preferably wherein (i) the channel of the nanopore comprises one or more non-native charged moieties and/or (ii) wherein said nanopore is comprised in a membrane and said system further comprises means for detecting electrical and/or optical signals across said membrane;
and
a peptide, polypeptide or protein at least 25 amino acid in length;
preferably wherein said peptide, polypeptide or protein comprises one or more post-translational modifications and/or one or more RNA splicing sites;
wherein said nanopore and/or said peptide, polypeptide or protein is present in a medium comprising a chaotropic agent;
and preferably wherein said system is configured such that when the peptide, polypeptide or protein is contacted with the nanopore an electroosmotic force across the nanopore is capable of causing the peptide, polypeptide or protein to translocate through the nanopore in a linearised state.
